(19) United States
(12) Patent Application Publication
(10) Pub. No.: US 2024/0382529 A1
(43) Pub. Date: Nov. 21, 2024

(54) COMPOSITIONS AND METHODS FOR TREATING SPINAL CORD INJURIES

(71) Applicant: ASTERIAS BIOTHERAPEUTICS, INC., Carlsbad, CA (US)

(72) Inventors: Gary HOGGE, San Francisco, CA (US); Rami SKALITER, Ness Ziona (IL); Jennifer BAHR-DAVIDSON, Carlsbad, CA (US); François BINETTE, Napa, CA (US); Kento ONISHI, Oakland, CA (US); Nathan C. MANLEY, San Jose, CA (US); Craig R. HALBERSTADT, Pleasanton, CA (US); Erik M. WHITELY, Concord, CA (US)

(21) Appl. No.: 18/833,784

(22) PCT Filed: Jan. 27, 2023
(86) PCT No.: PCT/US23/61506

§ 371 Date: Jul. 26, 2024

Related U.S. Application Data
(60) Provisional application No. 63/304,405, filed on Jan. 28, 2022.
(60) Provisional application No. 63/327,253, filed on Apr. 4, 2022.

(51) Int. Cl. A61K 35/30; A61M 5/14; A61M 5/158; A61M 5/168; C12N 5/0797

(52) U.S. Cl. CPC A61K 35/30; A61M 5/1413; A61M 5/158; A61M 5/16813; C12N 5/0623; A61M 2005/1586; A61M 2205/0288; A61M 2210/1003; C12N 2500/38; C12N 2501/155; C12N 2501/727; C12N 2506/02

(57) ABSTRACT

Provided herein are methods, compositions of matter, and devices for treating neurological diseases and illnesses, including spinal cord injury.

### CROSS REFERENCE TO RELATED APPLICATION

This application claims priority to U.S. Provisional Application No. 63/304,405 filed on Jan. 28, 2022 and U.S. Provisional Application No. 63/327,253 filed on Apr. 4, 2022, which is incorporated herein by reference in its entirety.

### REFERENCE TO A SEQUENCE LISTING

This application contains a Sequence Listing which has been submitted electronically and is hereby incorporated by reference in its entirety. Said Sequence Listing is in a file titled “058125-646001WO_SL_ST26.xml,” which was created on Jan. 27, 2023, and is approximately 3.43 KB in size.

### BACKGROUND

Spinal cord injury (SCI) is a devastating and currently untreatable condition, aside from symptomatic treatments for some of the resulting complications. Spinal cord injury results in complete or partial loss of motor, sensory, and autonomic function. As a result, patients often lose mobility and may be wheelchair-bound, in addition to suffering numerous medical complications. Over 12,000 Americans suffer a spinal cord injury (SCI) each year, and approximately 1.3 million people in the United States are estimated to be living with a spinal cord injury. Traumatic SCI most commonly impacts individuals in their twenties and thirties, resulting in a high-level of permanent disability in young and previously healthy individuals. Individuals with SCI not only have impaired limb function, but suffer from impaired bowel and bladder function, reduced sensation, spasticity, autonomic dysreflexia, thromboses, sexual dysfunction, increased infections, decubitus ulcers and chronic pain, which can each significantly impact quality of life, and can even be life threatening in some instances. The life expectancy of an individual suffering a cervical spinal cord injury at age 20 is 20-25 years lower than that of a similarly aged individual with no SCI (NSCISC Spinal Cord Injury Facts and Figures 2013). To date, there are no treatments approved by the United States Food and Drug Administration (FDA) to induce neurological recovery following spinal cord injury (SCI). Several interventions including glucocorticoids, modulation of voltage-gated channels, tetracycline antibiotics, and cell-based therapies have been studied in clinical trials, however, none to date have met critical registration endpoints.

The clinical effects of spinal cord injury vary with the site and extent of damage. The neural systems that may be permanently disrupted below the level of the injury not only involve loss of control of limb muscles and the protective roles of temperature and pain sensation, but impact the cardiovascular system, breathing, sweating, bowel control, bladder control, and sexual function (Anderson K D, Friden J, Lieber R L. Acceptable benefits and risks associated with surgically improving arm function in individuals living with cervical spinal cord injury. Spinal Cord. 2009 April; 47 (4): 334-8.) These losses lead to a succession of secondary problems, such as pressure sores and urinary tract infections that, until modern medicine, were rapidly fatal. Spinal cord injury often removes those unconscious control mechanisms that maintain the appropriate level of excitability in neural circuitry of the spinal cord. As a result, spinal motoneurons can become spontaneously hyperactive, producing debilitating stiffness and uncontrolled muscle spasms or spasticity. This hyperactivity can also cause sensory systems to produce chronic neurogenic pain and paresthesias, unpleasant sensations including numbness, tingling, aches, and burning. In recent polls of spinal cord injury patients, recovery of ambulatory function was not the highest ranked function that these patients desired to regain, but in many cases, relief from the spontaneous hyperactivity sequelae was paramount (Anderson K D, Friden J, Lieber R L. Acceptable benefits and risks associated with surgically improving arm function in individuals living with cervical spinal cord injury. Spinal Cord. 2009 April; 47 (4): 334-38).

There exists a need for treatments for spinal cord injury, and related pathologies.

### SUMMARY

The examples and embodiments presented herein describe human embryonic stem cell (hESC) derived cells for the treatment of spinal cord injuries (SCI) as described in greater detail herein.

For example, an oligodendrocyte progenitor cell (OPC) composition obtained in accordance with the present disclosure can be used in cellular therapy to improve one or more neurological functions in a subject in need of treatment. In an embodiment, an OPC cell population in accordance with the present disclosure can be injected, implanted, or otherwise delivered into a subject in need thereof. In an embodiment, a cell population in accordance with the present disclosure can be implanted or otherwise delivered into a subject in need thereof for treating spinal cord injury, stroke, or multiple sclerosis.

The LCTOPC1 is a cell population containing a mixture of oligodendrocyte progenitor cells and other characterized cell types obtained following directed differentiation of an established and well-characterized line of hESC. AST-OPC1 (formerly known as GRNOPC1) is a cell population that contains a mixture of oligodendrocyte progenitor cells (OPCs) and other characterized cell types that are obtained following differentiation of undifferentiated human embryonic stem cells (uhESCs).

Oligodendrocyte progenitor cells (OPCs) are a subtype of glial cells in the central nervous system (CNS) that arise in the ventricular zones of the brain and spinal cord and migrate throughout the developing CNS before maturing into oligodendrocytes. Mature oligodendrocytes produce the myelin sheath that insulates neuronal axons and remyelinate CNS lesions where the myelin sheath has been lost. Oligodendrocytes also contribute to neuroprotection through other mechanisms, including production of neurotrophic factors that promote neuronal survival (Wilkins et al., 2001 Glia 36 (1): 48-57; Dai et al., 2003 J Neurosci. 23 (13): 5846-53; Du and Dreyfus, 2002 J Neurosci Res. 68 (6): 647-54). Unlike most progenitor cells, OPCs remain abundant in the adult CNS where they retain the ability to generate new oligodendrocytes. Accordingly, OPCs and mature oligodendrocytes derived from OPCs are an important therapeutic target for demyelinating and dysmyelinating disorders (such as multiple sclerosis, adrenoleukodystrophy and adrenomyeloneuropathy), other neurodegenerative disorders (such as Alzheimer's disease, amyotrophic lateral sclerosis, and Huntington's disease) and acute neurological injuries (such as stroke and spinal cord injury (SCI)).

An OPC composition obtained in accordance with the present disclosure can be used in cellular therapy to improve one or more neurological functions in a subject in need of treatment. In an embodiment, an OPC cell population in accordance with the present disclosure can be injected or implanted into a subject in need thereof. In an embodiment, a cell population in accordance with the present disclosure can be implanted into a subject in need thereof for treating spinal cord injury, stroke, or multiple sclerosis.

In certain embodiments, the OPC1 composition is administered after the subject has suffered a traumatic spinal cord injury. In some embodiments, the OPC1 composition is administered between 14-90 days after the spinal cord injury, such as between 14-75 days after the spinal cord injury, such as between 14-60 days after the spinal cord injury, such as between 14-30 days after the injury, such as between 20-75 days after the injury, such as between 20-60 days after the injury, and such as between 20-40 days after the injury. In certain embodiments, the OPC1 composition is administered about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 5, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 days after the injury. In certain embodiments, the OPC1 composition is administered between 14 days and the lifetime of the subject.

Methods and compositions for obtaining a population of cells comprising dorsal neural progenitor cells (dNPCs) from undifferentiated human pluripotent stem cells can be found in WO/2020/154533, WO/2020/061371, U.S. Pat. No. 10,286,009, WO/2017/031092, WO/2017/173064 and WO/2018/053210, each of which are incorporated by reference in their entirety for all methods, compositions, cells, data, definitions, uses, and all other information provided therein.

In an aspect, a method of improving one or more neurological functions in a subject having a spinal cord injury (SCI) is provided, the method including: administering to the subject a first dose of a composition including human pluripotent stem cell-derived oligodendrocyte progenitor cells (OPCs); and optionally administering two or more doses of the composition. In some embodiments, the first dose of the composition is administered using a parenchymal spinal delivery (PSD) system. In some embodiments, the PSD is a system as described herein and as shown, for example, in FIGS. 39-41. See, also US Publication No: 2015/0224331, incorporated by reference herein in its entirety for all devices, methods, etc., described therein.

In some embodiments, the method further includes administering to the subject a second dose of the composition. In some embodiments, the method further includes administering to the subject a third dose of the composition. In some embodiments, each administration includes delivering, for example by injection, the composition into the spinal cord of the subject. In some embodiments, each administration includes delivering two or more fractions of a dose. In some embodiments, the second dose of the composition is administered using a parenchymal spinal delivery (PSD) system, e.g., the system as described herein and as shown, for example, in FIGS. 39-41. See, also US Publication No: 2015/0224331, incorporated by reference herein in its entirety for all devices, methods, etc., described therein.

In some embodiments, the third dose of the composition using a parenchymal spinal delivery (PSD) system, is as shown in FIGS. 39-41.

In some embodiments, the SCI is a subacute cervical SCI. In some embodiments, the SCI is a chronic cervical SCI. In some embodiments, the SCI is a subacute thoracic SCI. In some embodiments, the SCI is a chronic thoracic SCI. In some embodiments, the first dose, second dose, and/or third dose of the composition includes about 1×10⁶ to about 3×10⁷ OPC cells. In some embodiments, the first dose of the composition includes about 2×10⁶ OPC cells. In some embodiments, the first dose or the second dose of the composition includes about 1×10⁷ OPC cells. In some embodiments, the second dose or the third dose of the composition includes about 2×10⁷ OPC cells. In some embodiments, each of the first dose, second dose, and third dose of the composition are administered about 20 to about 45 days after the SCI. In some embodiments, each of the first dose, second dose, and third dose of the composition are administered about 14 to about 90 days after the SCI. In some embodiments, each of the first dose, second dose, and third dose of the composition are administered about 14 to about 75 days after the SCI. In some embodiments, each of the first dose, second dose, and third dose of the composition are administered about 14 to about 60 days after the SCI. In some embodiments, each of the first dose, second dose, and third dose of the composition are administered about 14 to about 30 days after the SCI. In some embodiments, each of the first dose, second dose, and third dose of the composition are administered about 20 to about 75 days after the SCI. In some embodiments, each of the first dose, second dose, and third dose of the composition are administered about 20 to about 60 days after the SCI. In some embodiments, each of the first dose, second dose, and third dose of the composition are administered about 20 to about 40 days after the SCI. In some embodiments, each of the first dose, second dose, and third dose of the composition are administered between about 14 days after the SCI and the lifetime of the subject. In some embodiments, the injection is performed in a caudal half of an epicenter of the SCI. In some embodiments, the injection is about 6 mm into the spinal cord of the subject. In some embodiments, the injection is about 5 mm into the spinal cord of the subject.

In another, aspect, a method of improving one or more neurological functions in a subject having a spinal cord injury (SCI) is provided, the method including: administering to the subject a dose of a composition including human pluripotent stem cell-derived oligodendrocyte progenitor cells (OPCs).

In some embodiments, the dose of the composition includes about 1×10⁶ to about 3×10⁷ OPC cells. In some embodiments, the dose of the composition includes about 2×10⁶ OPC cells. In some embodiments, the administration of the composition includes injecting, implanting, or otherwise delivering the composition into the spinal cord of the subject. In some embodiments, the dose of the composition is administered about 7 to about 14 days after the SCI. In some embodiments, the injection is performed in a caudal half of an epicenter of the SCI. In some embodiments, the injection is about 6 mm into the spinal cord of the subject. In some embodiments, the injection is about 5 mm into the spinal cord of the subject. In some embodiments, the SCI is a subacute thoracic SCI. In some embodiments, the SCI is a chronic thoracic SCI. In some embodiments, the SCI is a subacute cervical SCI. In some embodiments, the SCI is a chronic cervical SCI. In some embodiments, improving one or more neurological functions includes an improvement in ISNCSCI exam upper extremity motor score (UEMS). In some embodiments, the improvement in UEMS occurs within about 6 months, about 12 months, about 18 months, about 24 months or more after injection. In some embodiments, the improvement is an increase in UEMS of at least 10%, compared to baseline. In some embodiments, improving one or more neurological functions includes an improvement in lower extremity motor scores (LEMS). In some embodiments, the improvement in LEMS occurs within about 6 months, about 12 months, about 18 months, about 24 months or more after injection. In some embodiments, the improvement is at least one motor level improvement. In some embodiments, the improvement is at least two motor level improvement. In some embodiments, the improvement is on one side of the subject's body. In some embodiments, the improvement is on both sides of the subject's body. In some embodiments, the dose of the composition is administered about 14 to 90 days after the SCI. In some embodiments, the dose of the composition is administered about 14 to about 75 days after the SCI. In some embodiments, the dose of the composition is administered about 14 to about 60 days after the SCI. In some embodiments, the dose of the composition is administered about 14 to about 30 days after the SCI. In some embodiments, the dose of the composition is administered about 20 to about 75 days after the SCI. In some embodiments, the dose of the composition is administered about 20 to about 60 days after the SCI. In some embodiments, the dose of the composition is administered about 20 to about 40 days after the SCI. In some embodiments, the dose of the composition is administered between about 14 days after the SCI and the lifetime of the subject.

In another, aspect, a cell population is provided, the cell population including an increased proportion of cells positive for oligodendrocyte progenitor cell marker NG2 and reduced expression of non-OPC markers CD49f, CLDN6, and EpCAM, wherein the cell population is prepared according to the following method: culturing undifferentiated human embryonic stem cells (uhESC) in Glial Progenitor Medium including a MAPK/ERK inhibitor, a BMP signaling inhibitor, and Retinoic Acid to obtain glial-restricted cells; differentiating the glial-restricted cells into oligodendrocyte progenitor cells (OPCs) having an increased proportion of cells positive for oligodendrocyte progenitor cell marker NG2 and reduced expression of non-OPC markers CD49f, CLDN6, and EpCAM.

In some embodiments, the cell population is used in treating a thoracic spinal cord injury (SCI) in a subject. In some embodiments, the thoracic SCI is a subacute thoracic SCI. In some embodiments, the thoracic SCI is a chronic thoracic SCI. In some embodiments, the cell population is used in treating a cervical spinal cord injury (SCI) in a subject. In some embodiments, the cervical SCI is a subacute cervical SCI. In some embodiments, the cervical SCI is a chronic cervical SCI. In some embodiments, the composition is administered by implantation or other delivery method. In some embodiments, the composition is administered via injection to the subject after the SCI. In some embodiments, the injection is performed in a caudal half of an epicenter of the SCI. In some embodiments, the injection is about 6 mm into the spinal cord of the subject. In some embodiments, the injection is about 5 mm into the spinal cord of the subject. In some embodiments, the injection is performed about 14 to about 90 days after the SCI. In some embodiments, the injection is performed about 14 to about 75 days after the SCI. In some embodiments, the injection is performed about 14 to about 60 days after the SCI. In some embodiments, the injection is performed about 14 to about 30 days after the SCI. In some embodiments, the injection is performed about 20 to about 75 days after the SCI. In some embodiments, the injection is performed about 20 to about 60 days after the SCI. In some embodiments, the injection is performed about 20 to about 40 days after the SCI. In some embodiments, the injection is performed between about 14 days after the SCI and the lifetime of the subject.

In another, aspect, a method of improving one or more neurological functions in a subject having a spinal cord injury (SCI) is provided, the method including: administering to the subject a first dose of the cell population of claim 54; administering to the subject a second dose of the cell population; and optionally administering to the subject a third dose of the cell population.

In some embodiments, the SCI is a subacute cervical SCI. In some embodiments, the SCI is a chronic cervical SCI. In some embodiments, the SCI is a subacute thoracic SCI. In some embodiments, the SCI is a chronic thoracic SCI. In some embodiments, each of the first dose, second dose, and third dose of the composition are administered about 14 to about 90 days after the SCI. In some embodiments, each of the first dose, second dose, and third dose of the composition are administered about 14 to about 75 days after the SCI. In some embodiments, each of the first dose, second dose, and third dose of the composition are administered about 14 to about 60 days after the SCI. In some embodiments, each of the first dose, second dose, and third dose of the composition are administered about 14 to about 30 days after the SCI. In some embodiments, each of the first dose, second dose, and third dose of the composition are administered about 20 to about 75 days after the SCI. In some embodiments, each of the first dose, second dose, and third dose of the composition are administered about 20 to about 60 days after the SCI. In some embodiments, each of the first dose, second dose, and third dose of the composition are administered about 20 to about 40 days after the SCI. In some embodiments, each of the first dose, second dose, and third dose of the composition are administered between about 14 days after the SCI and the lifetime of the subject.

In embodiments, the composition is administered using a parenchymal spinal delivery (PSD) system. In embodiments, the PSD system comprises a device comprises a system as shown in any of FIGS. 39-41.

In embodiments, the PSD system includes a device including:
(a) a frame having an elongated body and a plurality of holders extending therefrom;
(b) a plurality of first magnets, each being fixedly attached to a holder;
(c) a tube having a first end and a second end, the tube being slidingly disposed within through-holes disposed in each holder and in each first magnet;
(d) a plurality of second magnets fixedly attached to an exterior surface of the tube; and
(e) a needle fixedly attached to the first end of the tube.

In embodiments, each of the first magnets and the each of the second magnets are disposed such that a north pole of one first magnet faces a north pole of one second magnet or a south pole of one first magnet faces a south pole of one second magnet, thereby providing a magnetic repulsive force upon which the tube floats.

In embodiments, the PSD system further includes a reservoir in fluid communication with the needle. In embodiments, the reservoir contains a composition as described herein.

In embodiments, the PSD system further includes a digital microinjector configured to control flow of the composition through the needle.

In embodiments, the device includes a stopper adjacent to the needle. In embodiments, the stopper prevents the needle from travelling further into the spinal cord.

In embodiments, the subject is not removed from ventilation during administration.

In embodiments, administering the composition includes:
(i) positioning the PSD system over the spinal cord of the subject;
(ii) lowering the needle into the spinal cord; and
(iii) delivering a dose of the composition to the spinal cord.

In embodiments, the needle and tube of the device float due to magnetic repulsive forces within the device, thereby compensating for spinal cord pulsation.

In embodiments, step (ii) includes lowering the needle until the stopper rests on the spinal cord.

In some aspects is provided a parenchymal spinal delivery (PSD) system including: (i) a device as shown in any of FIGS. 39-41 or described herein which comprises a needle; and
(ii) a reservoir in fluid communication with the needle, the reservoir containing a composition as described herein.

In embodiments, the device includes:
(a) a frame having an elongated body and a plurality of holders extending therefrom;
(b) a plurality of first magnets, each being fixedly attached to a holder;
(c) a tube having a first end and a second end, the tube being slidingly disposed within through-holes disposed in each holder and in each first magnet;
(d) a plurality of second magnets fixedly attached to an exterior surface of the tube; and
(e) the needle fixedly attached to the first end of the tube.

In embodiments, each of the first magnets and the each of the second magnets are disposed such that a north pole of one first magnet faces a north pole of one second magnet or a south pole of one first magnet faces a south pole of one second magnet, thereby providing a magnetic repulsive force upon which the tube floats. In embodiments, the system includes a microinjector or microinjection pump configured to control flow of the composition through the needle. In embodiments, the device includes an XYZ manipulator for manipulating a position of the needle. In embodiments, the device includes a stopper adjacent to the needle. In embodiments, the stopper prevents the needle from travelling further into a spinal cord.

### BRIEF DESCRIPTION OF THE DRAWINGS

Embodiments will now be described, by way of example only, with reference to the accompanying drawings, in which:

FIG. 1 is a Phase-1 clinical trial schematic timeline.

FIG. 2 is schematic for patient screening, treatment, and follow-up during a Phase-1 clinical trial (CONSORT flow diagram).

FIG. 3 is a diagram illustrating the International Standards for Neurological Classification of Spinal Cord Injury (ISNCSCI) screening and at 5-year follow-up (* one ISNSCI performed at 4-year). In the figure white denotes normal motor and/or sensation, left-to-right hatching denotes absent motor and/or sensation, right-to-left hatching represents sensation that is present but abnormal.

FIG. 4 is an example questionnaire administered for the long-term protocol; annual visits were required in years 2-5. Subsequent to the year 5 annual visit, follow-up was by annual phone questionnaires.

FIG. 5 is a study schematic of subjects.

FIG. 6 is a clinical trial schematic timeline.

FIG. 7 is a schematic for patient screening and treatment during a clinical trial.

FIG. 8 is a schematic of the cohort structure and enrollment progression of a clinical trial consistent with the implementations of the present disclosure.

FIG. 9 is another Phase-1 clinical trial schematic timeline consistent with implementations of the present disclosure.

FIG. 10 is an overview of two example cell manufacturing processes consistent with implementations of the present disclosure.

FIG. 11 is a flow chart of a signaling sequence schematic of a cell differentiation process consistent with implementations of the present disclosure.

FIG. 12 is a flowchart of a production process flow consistent with implementations of the present disclosure.

FIG. 13 is a schematic of SCI treatment objectives. Loss of movement is the primary feature of a spinal cord injury. Higher-level injuries result in more extensive impairments. Gains in motor function, particularly in the upper extremities, can provide significant benefits in self-care and lower costs of care. The goal of the cell therapy is to provide additional arm, hand, and finger function, increasing independence and quality of life.

FIG. 14 is a schematic describing OPC1 cells for SCIs. Replacing oligodendrocytes may provide additional upper limb and finger function and improve the quality of life for patients. OPC1 is comprised of OPCs (oligodendrocyte progenitor cells). OPCs are precursors to Oligodendrocytes, the myelinating cells of the central nervous system, which provide insulation to nerve axons in the form of a myelin sheath. Myelin is necessary for proper function of neurons. OPC1 cells are injected into the spinal cord at the injury site.

FIG. 15 is a schematic of the three major physiologically relevant functional activities of OPC1 cells.

FIG. 16 is an image showing the prevention of cavitation by OPC1 cells.

FIG. 17 is an image showing OPC1: hESC-Derived Oligodendrocyte Progenitor Cells (OPCs). OPC1 is a cryopreserved allogeneic cell population. It is derived from an NIH-registered human embryonic stem cell line (hESC). It is a characterized composition of cells, consisting of oligodendrocyte progenitors, neural progenitors, infrequent mature neural cells, and rare other characterized cell types. OPC1 has three identified functions: it produces neurotrophic factors, it induces remyelination, it induces vascularization. It can be used for “off the shelf” administration. Its first indication is for spinal cord injury. There are potential line extensions in other neurodegenerative diseases.

FIG. 18 is a schematic showing the OPC1 injection procedure. Injections are performed using a table-mounted syringe positioning device (SPD). The composition is administered via direct intraparenchymal injection into the spinal cord lesion. The dosage consists of a single 50 μL injection for both the 2M & 10M doses and two injections for the 20M dose. There are no intraoperative complications.

FIG. 19 is an image showing the International Standards for Neurological Classification of Spinal Cord Injury.

FIG. 20 is a schematic showing the thoracic study schema.

FIG. 21 is an image showing thoracic trial adverse events and serious adverse events.

FIG. 22 is an image showing thoracic trial serious adverse events.

FIG. 23 is a table showing thoracic trial ISNCSCI Exams, Baseline, Year 1 and 5 Year. The Table presents total sensory score (TSS), upper extremity motor score (UEMS), lower extremity motor score (LEMS), neurological level of injury (NLI), motor NLI, sensory zone of partial preservation (ZPP), Motor ZPP, and ASIA impairment Score (AIS) grade at baseline, year 1 and year 5. All five participants were AIS Grade A at enrollment and there were no conversions to AIS B. The highest single and lowest NLI enrolled in the study were T3 and T8 respectively. Only the individual with the T3 NLI improved to T4 with a sensory ZPP initially at T4 bilaterally noted to improve to T5 on the left and T6 on the right at one year follow up. In total three of five participants experienced at least 1 level of improvement in their ZPP. ND=Unable to determine.

FIG. 24 is an image showing thoracic trial sensory outcomes, with diagrammatic representation of sensory function of each patient at baseline and at 5 years post OPC1 administration. In the figure white denotes normal motor and/or sensation, left-to-right hatching denotes absent motor and/or sensation, right-to-left hatching represents sensation that is present but abnormal.

FIG. 25 is an image showing International Standards for Neurological Classification of Spinal Cord Injury.

FIG. 26 is an image showing benefit from a 2 motor level improvement. Motor level gains translate into clinically meaningful improvements in self-care and reductions in cost of care.

FIG. 27 is a schematic showing the cervical clinical trial study design.

FIG. 28 is an image showing year 1 follow-up status for cervical trial.

FIG. 29 is a schematic showing cervical trial schema.

FIG. 30 is an image showing cervical trial summary of adverse events. The majority of cervical trial adverse events were mild to moderate in severity. 8/11 AEs related to Tacrolimus were hypomagnesemia; 20 AEs related to the injection procedure (nausea, vomiting, postoperative wound infection, autonomic dysreflexia, procedural pain, myalgia, neck pain, headache, incision drainage, cerebrospinal fluid retention, and cerebrospinal fluid leakage).

FIG. 31 is an image showing the motor function gains from the cervical trial; 2/3 (68%) had 1 level and ¼ (25%) had 2 levels. Regarding spontaneous recovery, Steeves et al. reported, the “Proportion (%) of people living with cervical (C4-C7) sensorimotor complete (AIS A) SCI spontaneously recovering 1 or >2 upper extremity motor levels [on either right or left side, whichever is greater] after [48 weeks post] injury [is 68% for one level and 26% for two levels, respectively].”

FIG. 32 is a schematic showing upper extremity motor score (UEMS) recovery in cohorts 2-5.

FIG. 33 is an image showing cervical trial evaluation of change in UEMS (12 months post-injection versus key variables).

FIG. 34 is an image showing analysis of patients with least UEMS recovery from the cervical trial. C4 or cord compressions occurred in 5 of the 7 worst patient outcomes and both issues can be addressed in the next trial. Two patients had cord compression after OPC1 injection (17 and 23 at Day 30 and Day 7). Patients 15, 23, 18 had a C4 (highest/most severe) injury level at baseline. Patient 15 also had a hematoma in the spinal cord at baseline and a failed graft.

FIG. 35 displays images from subject 15 (cohort 3) with a hematoma in the spinal cord, showing that some lesions may be too severe for OPC1 cell survival.

FIG. 36 displays images from subject 17 (cohort 4), showing cord compression at day 30 in the cervical trial.

FIG. 37 is a schematic showing that the 12 month MRI results support durable engraftment of OPC1 cells. Cavitation is estimated to occur in ˜80% of spinal cord injury patients meeting cervical trial inclusion criteria. 96% ( 24/25) of participants had serial MRI scans at 12 months that indicated no sign of a lesion cavity. The MRI results were consistent with formation of a tissue matrix at injury site, which is believed to be supportive evidence showing that OPC1 cells have durably engrafted to help prevent cavitation at the injury site (see Wirth et al. Exp Neurology 1995).

FIG. 38 is an image showing the original syringe positioning device in the cervical trial.

FIG. 39 is an image showing a parenchymal spinal delivery (PSD) system.

FIG. 40 is an image showing an overview of the PSD system.

FIG. 41 is an image showing an overview of the PSD system.

FIG. 42 is a bar graph showing OPC1 manufacturing improvements.

FIG. 43 is a graphic showing the range of motion of a magnetic needle (DW-29002).

FIG. 44 shows the magnetic needle test setup.

FIGS. 45A and 45B illustrate the characteristic curve of repulsion load vs. displacement. FIG. 45A is a graph showing the magnetic needle repulsion force. FIG. 45B is a table illustrating the displacement of the needle.

FIGS. 46A-46D illustrate a video analysis of magnetic needle deflections during simulated use in the pig spine model. FIG. 46A shows the initial penetration. FIG. 46B shows the final penetration. FIG. 46C shows the needle at resting position. FIG. 46D shows the amount of movement from aspiration.

FIG. 47 is a graph showing the force and deflection at points of use.

FIG. 48 shows the initial fixture setup and loading of magnetic needle assembly.

FIG. 49 shows tightening screws holding the syringe mount fastener.

FIG. 50 illustrates the magnetic needle assembly.

FIG. 51 illustrates the test frame.

FIG. 52 is a graph showing the load-displacement curve for sample B1.

FIG. 53 is a graph showing the load-displacement curve for sample B2.

FIG. 54 is a graph showing the load-displacement curve for sample B3.

FIG. 55 is a graph showing the load-displacement curve for sample B4.

FIG. 56 is a graph showing the load-displacement curve for sample B5.

FIG. 57 is a graph showing the load-displacement curve for sample B6.

FIG. 58 is a graph showing the load-displacement curve for sample B7.

FIG. 59 is a graph showing the load-displacement curve for sample B8.

FIG. 60 is a graph showing the load-displacement curve for sample B9.

FIG. 61 is a graph showing the load-displacement curve for sample B10.

FIG. 62 is a graph showing the load-displacement curve for sample B11.

FIG. 63 is a graph showing the load-displacement curve for sample B12.

FIG. 64 is a graph showing the load-displacement curve for sample B13.

FIG. 65 is a graph showing the load-displacement curve for sample B14.

FIG. 66 is a graph showing the load-displacement curve for sample B15.

### DETAILED DESCRIPTION

Before the present compositions and methods are described, it is to be understood that the present disclosure is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular aspects of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

All publications, patent applications, patents and other references cited herein are incorporated by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

Methods disclosed herein can comprise one or more steps or actions for achieving the described method. The method steps and/or actions may be interchanged with one another without departing from the scope of the present invention. In other words, unless a specific order of steps or actions is required for proper operation of the embodiment, the order and/or use of specific steps and/or actions may be modified without departing from the scope of the present invention.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a percentages, density, volume and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “AST-OPC1” refers to a specific, characterized, in vitro differentiated cell population containing a mixture of oligodendrocyte progenitor cells (OPCs) and other characterized cell types obtained from undifferentiated human embryonic stem cells (uhESCs) according to specific differentiation protocols disclosed herein.

Compositional analysis of AST-OPC1 by immunocytochemistry (ICC), flow cytometry, and quantitative polymerase chain reaction (qPCR) demonstrates that the cell population is comprised primarily of neural lineage cells of the oligodendrocyte phenotype. Other neural lineage cells, namely astrocytes and neurons, are present at low frequencies. The only non-neural cells detected in the population are epithelial cells. Mesodermal, endodermal lineage cells and uhESCs are routinely below quantitation or detection of the assays.

The term “oligodendrocyte progenitor cells” (OPCs), as used herein, refers to cells of neuroectoderm/glial lineage having the characteristics of a cell type found in the central nervous system, capable of differentiating into oligodendrocytes. These cells typically express the characteristic markers Nestin, NG2 and PDGF-Ra.

The terms “treatment,” “treat” “treated,” or “treating,” as used herein, can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results may include, but are not limited to one or more of the following: alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response. Treatment also includes prolonging survival as compared to expected survival if not receiving treatment.

The term “subject,” as used herein includes, but is not limited to, humans, nonhuman primates and non-human vertebrates such as wild, domestic and farm animals including any mammal, such as cats, dogs, cows, sheep, pigs, horses, rabbits, rodents such as mice and rats. In some embodiments, the term “subject,” refers to a male. In some embodiments, the term “subject,” refers to a female.

As used herein, “implantation” or “transplantation” refers to the administration of a cell population into a target tissue using a suitable delivery technique, (e.g., using an injection device, implantation device, or other delivery device). In some examples, the administration of the composition uses a parenchymal spinal delivery (PSD) system. In some examples, the administration of the composition uses the PSD system described herein. In some examples, the administration of the composition uses the PSD described in U.S. Publication No: 2015/0224331, incorporated by reference herein in its entirety.

As used herein, “engraftment” and “engrafting” refer to incorporation of implanted tissue or cells (i.e. “graft tissue” or “graft cells”) into the body of a subject. The presence of graft tissue or graft cells at or near the implantation site 180 days or later, post implantation, is indicative of engraftment. In certain embodiments, imaging techniques (such as, e.g. MRI imaging), can be used to detect the presence of graft tissue.

As used herein, “allogeneic” and “allogeneically derived” refer to cell populations derived from a source other than the subject and hence genetically non-identical to the subject. In certain embodiments, allogeneic cell populations are derived from cultured pluripotent stem cells. In certain embodiments, allogeneic cell populations are derived from hESCs. In other embodiments, allogeneic cell populations are derived from induced pluripotent stem (iPS) cells. In yet other embodiments, allogeneic cell populations are derived from primate pluripotent (pPS) cells.

As used herein, “parenchymal cavitation” refers to formation of a lesion or cavity within a CNS injury site or proximate to a CNS injury site, in an area normally occupied by parenchymal CNS tissue. The cavities or lesions can be filled with extracellular fluid and may contain macrophages, small bands of connective tissue and blood vessels.

The terms “central nervous system” and “CNS” as used interchangeably herein refer to the complex of nerve tissues that control one or more activities of the body, which include but are not limited to, the brain and the spinal cord in vertebrates.

The term “decorin” as used herein refers to a proteoglycan that, in humans, is encoded by the DON gene. Decorin is a small cellular or pericellular matrix proteoglycan, and the protein is a component of connective tissue, binds to type I collagen fibrils, and plays a role in matrix assembly.

The term “chronic” as used herein includes, but is not intended to be limited to, a condition occurring in a subject over a time period occurring between 90 days after an injury and the lifetime of a subject.

The term “subacute” as used herein includes, but is not intended to be limited to, a condition occurring in a subject over a time period of between 14 days and 90 days after an injury.

There are multiple pathologies observed in the injured spinal cord due to the injury itself and subsequent secondary effects due to edema, hemorrhage and inflammation (Kakulas B A. The applied neuropathology of human spinal cord injury. Spinal Cord. 1999 February; 37 (2): 79-88). These pathologies include the severing of axons, demyelination, parenchymal cavitation and the production of ectopic tissue such as fibrous scar tissue, gliosis, and dystrophic calcification (Anderson D K, Hall E D. Pathophysiology of spinal cord trauma. Ann Emerg Med. 1993 June; 22 (6): 987-92; Norenberg M D, Smith J, Marcillo A. The pathology of human spinal cord injury: defining the problems. J. Neurotrauma. 2004 April; 21 (4): 429-40). Oligodendrocytes, which provide both neurotrophic factor and myelination support for axons are susceptible to cell death following SCI and therefore are an important therapeutic target (Almad A, Sahinkaya F R, Mctigue D M. Oligodendrocyte fate after spinal cord injury. Neurotherapies 2011 8 (2): 262-73). Replacement of the oligodendrocyte population could both support the remaining and damaged axons and also remyelinate axons to promote electrical conduction (Cao Q, He Q, Wang Yet et al. Transplantation of ciliary neurotrophic factor-expressing adult oligodendrocyte precursor cells promotes remyelination and functional recovery after spinal cord injury. J. Neurosci. 2010 30 (8): 2989-3001).

Oligodendrocyte progenitor cells (OPCs) are a subtype of glial cells in the central nervous system (CNS) that arise in the ventricular zones of the brain and spinal cord and migrate throughout the developing CNS before maturing into oligodendrocytes. Mature oligodendrocytes produce the myelin sheath that insulates neuronal axons and remyelinate CNS lesions where the myelin sheath has been lost. Oligodendrocytes also contribute to neuroprotection through other mechanisms, including production of neurotrophic factors that promote neuronal survival (Wilkins et al., 2001 Glia 36 (1): 48-57; Dai et al., 2003 J Neurosci. 23 (13): 5846-53; Du and Dreyfus, 2002 J Neurosci Res. 68 (6): 647-54). Additionally, OPCs are known to produce Decorin, a secreted factor which has been shown to suppress CNS scarring (Esmaeili, Berry et al, 2014, Gubbiotti, Vallet et al. 2016). Unlike most progenitor cells, OPCs remain abundant in the adult CNS where they retain the ability to generate new oligodendrocytes. Accordingly, OPCs and mature oligodendrocytes derived from OPCs are an important therapeutic target for demyelinating and dysmyelinating disorders (such as multiple sclerosis, adrenoleukodystrophy and adrenomyeloneuropathy), other neurodegenerative disorders (such as Alzheimer's disease, amyotrophic lateral sclerosis, and Huntington's disease) and acute neurological injuries (such as stroke and spinal cord injury (SCI)).

### Propagation and Culture of Undifferentiated Pluripotent Stem Cells

In certain embodiments, the present disclosure provides methods to produce large numbers of highly pure, characterized oligodendrocyte progenitor cells from pluripotent stem cells. Derivation of oligodendrocyte progenitor cells (OPCs) from pluripotent stem cells according to the methods of the invention provides a renewable and scalable source of OPCs for a number of important therapeutic, research, development, and commercial purposes, including treatment of acute spinal cord injury.

Methods of propagation and culture of undifferentiated pluripotent stem cells have been previously described. With respect to tissue and cell culture of pluripotent stem cells, the reader may wish to refer to any of numerous publications available in the art, e.g., Teratocarcinomas and Embryonic Stem cells: A Practical Approach (E. J. Robertson, Ed., IRL Press Ltd. 1987); Guide to Techniques in Mouse Development (P. M. Wasserman et al., Eds., Academic Press 1993); Embryonic Stem Cell Differentiation in Vitro (M. V. Wiles, Meth. Enzymol. 225:900, 1993); Properties and Uses of Embryonic Stem Cells: Prospects for Application to Human Biology and Gene Therapy (P. D. Rathjen et al., Reprod. Fertil. Dev. 10:31, 1998; and R. I. Freshney, Culture of Animal Cells, Wiley-Liss, New York, 2000).

In certain embodiments, a method can be carried out on a pluripotent stem cell line. In other embodiments, a method can be carried out on an embryonic stem cell line. In an embodiment, a method can be carried out on a plurality of undifferentiated stem cells that are derived from an H1, H7, H9, H13, or H14 cell line. In another embodiment, undifferentiated stem cells can be derived from an induced pluripotent stem cell (iPS) line. In another embodiment, a method can be carried out on a primate pluripotent stem (pPS) cell line. In yet another embodiment, undifferentiated stem cells can be derived from parthenotes, which are embryos stimulated to produce hESCs without fertilization.

In one embodiment, undifferentiated pluripotent stem cells can be maintained in an undifferentiated state without added feeder cells (see, e.g., (2004) Rosler et al., Dev. Dynam. 229:259). Feeder-free cultures are typically supported by a nutrient medium containing factors that promote proliferation of the cells without differentiation (see, e.g., U.S. Pat. No. 6,800,480). In one embodiment, conditioned media containing such factors can be used. Conditioned media can be obtained by culturing the media with cells secreting such factors. Suitable cells include, but are not limited to, irradiated (4,000 Rad) primary mouse embryonic fibroblasts, telomerized mouse fibroblasts, or fibroblast-like cells derived from pPS cells (U.S. Pat. No. 6,642,048). Medium can be conditioned by plating the feeders in a serum free medium, such as knock-out DMEM supplemented with 20% serum replacement and 4 ng/mL bFGF. Medium that has been conditioned for 1-2 days can be supplemented with further bFGF, and used to support pPS cell culture for 1-2 days (see. e.g., WO 01/51616; Xu et al., (2001) Nat. Biotechnol. 19:971).

Alternatively, fresh or non-conditioned medium can be used, which has been supplemented with added factors (such as, e.g., a fibroblast growth factor or forskolin) that promote proliferation of the cells in an undifferentiated form. Non-limiting examples include a base medium like X-VIVO™ 10 (Lonza, Walkersville, Md.) or QBSF™-60 (Quality Biological Inc. Gaithersburg, Md.), supplemented with bFGF at 40-80 ng/mL, and optionally containing SCF (15 ng/mL), or Flt3 ligand (75 ng/mL) (see, e.g., Xu et al., (2005) Stem Cells 23 (3): 315). These media formulations have the advantage of supporting cell growth at 2-3 times the rate in other systems (see, e.g., WO 03/020920). In one embodiment, undifferentiated pluripotent cells such as hESCs, can be cultured in a media comprising bFGF and TGFP. Non-limiting example concentrations of bFGF include about 80 ng/ml. Non-limiting example concentrations of TGFP include about 0.5 ng/ml.

In one embodiment, undifferentiated pluripotent cells can be cultured on a layer of feeder cells, typically fibroblasts derived from embryonic or fetal tissue (Thomson et al. (1998) Science 282:1145). Feeder cells can be derived, inter alia, from a human or a murine source. Human feeder cells can be isolated from various human tissues, or can be derived via differentiation of human embryonic stem cells into fibroblast cells (see, e.g., WO 01/51616). In one embodiment, human feeder cells that can be used include, but are not limited to, placental fibroblasts (see, e.g., Genbacev et al. (2005) Fertil. Steril. 83 (5): 1517), fallopian tube epithelial cells (see, e.g., Richards et al. (2002) Nat. Biotechnol., 20:933), foreskin fibroblasts (see, e.g., Amit et al. (2003) Biol. Reprod. 68:2150), and uterine endometrial cells (see, e.g., Lee et al. (2005) Biol. Reprod. 72 (1): 42).

Various solid surfaces can be used in the culturing of undifferentiated pluripotent cells. Those solid surfaces include, but are not limited to, standard commercially available cell culture plates, such as 6-well, 24-well, 96-well, or 144-well plates. Other solid surfaces include, but are not limited to, microcarriers and disks. Solid surfaces suitable for growing undifferentiated pluripotent cells can be made of a variety of substances including, but not limited to, glass or plastic such as polystyrene, polyvinylchloride, polycarbonate, polytetrafluorethylene, melinex, thermanox, or combinations thereof. In one embodiment, suitable surfaces can comprise one or more polymers, such as, e.g., one or more acrylates. In one embodiment, a solid surface can be three-dimensional in shape. Non-limiting examples of three-dimensional solid surfaces are described, e.g., in U.S. Patent Pub. No. 2005/0031598.

In one embodiment, undifferentiated stem cells can be grown under feeder-free conditions on a growth substrate. In one embodiment, a growth substrate can be Matrigel® (e.g., Matrigel® or Matrigel® GFR), recombinant Laminin, or Vitronectin. In another embodiment, undifferentiated stem cells can be subcultured using various methods such as using collagenase, or such as manual scraping. In another embodiment, undifferentiated stem cells can be subcultured using non-enzymatic means, such as 0.5 mM EDTA in PBS, or such as using ReLeSR™. In an embodiment, a plurality of undifferentiated stem cells are seeded or subcultured at a seeding density that allows the cells to reach confluence in about three to about ten days. In an embodiment, the seeding density can range from about 6.0×10³ cells/cm² to about 5.0×10⁵ cells/cm², such as about 1.0×10⁴ cells/cm², such as about 5.0×10⁴ cells/cm², such as about 1.0×10⁵ cells/cm², or such as about 3.0×10⁵ cells/cm² of growth surface. In another embodiment, the seeding density can range from about 6.0×10³ cells/cm² to about 1.0×10⁴ cells/cm² of growth surface, such as about 6.0×10³ cells/cm² to about 9.0×10³ cells/cm², such as about 7.0×10³ cells/cm² to about 1.0×10⁴ cells/cm², such as about 7.0×10³ cells/cm² to about 9.0×10³ cells/cm², or such as about 7.0×10³ cells/cm² to about 8.0×10³ cells/cm² of growth surface. In yet another embodiment the seeding density can range from about 1.0×10⁴ cells/cm² to about 1.0×10⁵ cells/cm² of growth surface, such as about 2.0×10⁴ cells/cm² to about 9.0×10⁴ cells/cm², such as about 3.0×10⁴ cells/cm² to about 8.0×10⁴ cells/cm², such as about 4.0×10⁴ cells/cm² to about 7.0×10⁴ cells/cm², or such as about 5.0×10⁴ cells/cm² to about 6.0×10⁴ cells/cm² of growth surface. In an embodiment, the seeding density can range from about 1.0×10⁵ cells/cm² to about 5.0×10⁵ cells/cm² of growth surface, such as about 1.0×10⁵ cells/cm² to about 4.5×10⁵ cells/cm², such as about 1.5×10⁵ cells/cm² to about 4.0×10⁵ cells/cm², such as about 2.0×10⁵ cells/cm² to about 3.5×10⁵ cells/cm², or such as about 2.5×10⁵ cells/cm² to about 3.0×10⁵ cells/cm² of growth surface.

Any of a variety of suitable cell culture and sub-culturing techniques can be used to culture cells in accordance with the present disclosure. For example, in one embodiment, a culture medium can be exchanged at a suitable time interval. In one embodiment, a culture medium can be completely exchanged daily, initiating about 2 days after sub-culturing of the cells. In another embodiment, when a culture reaches about 90% colony coverage, a surrogate flask can be sacrificed and enumerated using one or more suitable reagents, such as, e.g., Collagenase IV and 0.05% Trypsin-EDTA in series to achieve a single cell suspension for quantification. In an embodiment, a plurality undifferentiated stem cells can then be subcultured before seeding the cells on a suitable growth substrate (e.g., Matrigel® GFR) at a seeding density that allows the cells to reach confluence over a suitable period of time, such as, e.g., in about three to ten days. In one embodiment, undifferentiated stem cells can be subcultured using Collagenase IV and expanded on a recombinant laminin matrix. In one embodiment, undifferentiated stem cells can be subcultured using Collagenase IV and expanded on a Matrigel® matrix. In one embodiment, undifferentiated stem cells can be subcultured using ReLeSR™ and expanded on a Vitronectin matrix.

In one embodiment, the seeding density can range from about 6.0×10³ cells/cm² to about 5.0×10⁵ cells/cm², such as about 1.0×10⁴ cells/cm², such as about 5.0×10⁴ cells/cm², such as about 1.0×10⁵ cells/cm², or such as about 3.0×10⁵ cells/cm² of growth surface. In another embodiment, the seeding density can range from about 6.0×10³ cells/cm² to about 1.0×10⁴ cells/cm² of growth surface, such as about 6.0×10³ cells/cm² to about 9.0×10³ cells/cm², such as about 7.0×10³ cells/cm² to about 1.0×10⁴ cells/cm², such as about 7.0×10³ cells/cm² to about 9.0×10³ cells/cm², or such as about 7.0×10³ cells/cm² to about 8.0×10³ cells/cm² of growth surface. In yet another embodiment, the seeding density can range from about 1.0×10⁴ cells/cm² to about 1.0×10⁵ cells/cm² of growth surface, such as about 2.0×10⁴ cells/cm² to about 9.0×10⁴ cells/cm², such as about 3.0×10⁴ cells/cm² to about 8.0×10⁴ cells/cm², such as about 4.0×10⁴ cells/cm² to about 7.0×10⁴ cells/cm², or such as about 5.0×10⁴ cells/cm² to about 6.0×10⁴ cells/cm² of growth surface. In an embodiment, the seeding density can range from about 1.0×10⁵ cells/cm² to about 5.0×10⁵ cells/cm² of growth surface, such as about 1.0×10⁵ cells/cm² to about 4.5×10⁵ cells/cm², such as about 1.5×10⁵ cells/cm² to about 4.0×10⁵ cells/cm², such as about 2.0×10⁵ cells/cm² to about 3.5×10⁵ cells/cm², or such as about 2.5×10⁵ cells/cm² to about 3.0×10⁵ cells/cm² of growth surface.

### Oligodendrocyte Progenitor Cell Compositions

As discussed above, the present disclosure provides compositions comprising a population of oligodendrocyte progenitor cells (OPCs) as well as methods of making and using the same from use in the treatment of acute spinal cord injury and other related CNS conditions. In certain embodiments, the OPCs of the present disclosure are capable of producing and secreting one or more biological factors that may augment neural repair.

In one embodiment, a cell population can have a common genetic background. In an embodiment, a cell population may be derived from one host. In an embodiment, a cell population can be derived from a pluripotent stem cell line. In another embodiment, a cell population can be derived from an embryonic stem cell line. In an embodiment, a cell population can be derived from a hESC line. In an embodiment, a hESC line can be an H1, H7, H9, H13, or H14 cell line. In another embodiment, a cell population can be derived from an induced pluripotent stem cell (iPS) line. In an embodiment a cell population can be derived from a subject in need thereof (e.g., a cell population can be derived from a subject that is in need to treatment). In yet another embodiment, a hESC line can be derived from parthenotes, which are embryos stimulated to produce hESCs without fertilization.

In certain embodiments, the OPCs of the present disclosure express one or more markers chosen from Nestin, NG2, Olig 1 and PDGF-Ra. In certain embodiments, the OPCs of the present disclosure express all of the markers Nestin, NG2, Olig 1 and PDGF-Ra.

In certain embodiments, the OPCs of the present disclosure are capable of secreting one or more biological factors. In certain embodiments, the one or more biological factors secreted by the OPCs of the present disclosure may promote, without limitation, neural repair, axonal outgrowth and/or glial differentiation, or any combination thereof. In some embodiments, the OPCs are capable of secreting one or more factors that stimulate axonal outgrowth. In some embodiments, the OPCs are capable of secreting one or more factors promoting glial differentiation by neural precursor cells. In some embodiments, the OPCs are capable of secreting one or more chemoattractants for neural precursor cells. In some embodiments, the OPCs are capable of secreting one or more inhibitors of matrix metalloproteinases. In some embodiments, the OPCs are capable of secreting one or more factors inhibiting cell death after spinal cord injury. In some embodiments, the OPCs are capable of secreting one or more factors that are upregulated post-cellular injury and that aid in the clearance of misfolded proteins.

In certain embodiments, the OPCs are capable of producing and secreting one or more biological factors selected from MCP-1, Clusterin, ApoE, TIMP1 and TIMP2. In further embodiments the OPCs are capable of producing and secreting MCP-1 and one or more of the factors selected from Clusterin, ApoE, TIMP1 and TIMP2. In yet further embodiments, the OPCs are capable of producing and secreting all of the factors MCP-1, Clusterin, ApoE, TIMP1 and TIMP2.

In an embodiment, a biological factor can be secreted by a composition comprising a population of OPCs at a concentration of more than about 50 pg/ml, such as more than about 100 pg/ml, such as more than about 200 pg/ml, such as more than about 300 pg/ml, such as more than about 400 pg/ml, such as more than about 500 pg/ml, such as more than about 1,000 pg/ml, such as more than about 2,000 pg/ml, such as more than about 3,000 pg/ml, such as more than about 4,000 pg/ml, such as more than about 5,000 pg/ml, such as more than about 6,000 pg/ml, or such as more than about 7,000 pg/ml. In certain embodiments, a biological factor can be secreted by a composition comprising a population of cells comprising OPCs at a concentration ranging from about 50 pg/ml to about 100,000 pg/ml, such as about 100 pg/ml, such as about 150 pg/ml, such as about 200 pg/ml, such as about 250 pg/ml, such as about 300 pg/ml, such as about 350 pg/ml, such as about 400 pg/ml, such as about 450 pg/ml, such as about 500 pg/ml, such as about 550 pg/ml, such as about 600 pg/ml, such as about 650 pg/ml, such as about 700 pg/ml, such as about 750 pg/ml, such as about 800 pg/ml, such as about 850 pg/ml, such as about 900 pg/ml, such as about 1,000 pg/ml, such as about 1,500 pg/ml, such as about 2,000 pg/ml, such as about 2,500 pg/ml, such as about 3,000 pg/ml, such as about 3,500 pg/ml, such as about 4,000 pg/ml, such as about 4,500 pg/ml, such as about 5,000 pg/ml, such as about 5,500 pg/ml, such as about 6,000 pg/ml, such as about 6,500 pg/ml, such as about 7,000 pg/ml, such as about 7,500 pg/ml, such as about 8,000 pg/ml, such as about 8,500 pg/ml, such as about 9,000 pg/ml, such as about 10,000 pg/ml, such as about 15,000 pg/ml, such as about 20,000 pg/ml, such as about 25,000 pg/ml, such as about 30,000 pg/ml, such as about 35,000 pg/ml, such as about 40,000 pg/ml, such as about 45,000 pg/ml, such as about 50,000 pg/ml, such as about 55,000 pg/ml, such as about 60,000 pg/ml, such as about 65,000 pg/ml, such as about 70,000 pg/ml, such as about 75,000 pg/ml, such as about 80,000 pg/ml, such as about 85,000 pg/ml, such as about 90,000 pg/ml, such as about 95,000 pg/ml.

In certain embodiments, a biological factor can be secreted by a composition comprising a population of cells comprising OPCs at a concentration ranging from about 1,000 pg/ml to about 10,000 pg/ml, such as about 1,000 pg/ml to about 2,000 pg/ml, such as about 2,000 pg/ml to about 3,000 pg/ml, such as about 3,000 pg/ml to about 4,000 pg/ml, such as about 4,000 pg/ml to about 5,000 pg/ml, such as about 5,000 pg/ml to about 6,000 pg/ml, such as about 6,000 pg/ml to about 7,000 pg/ml, such as about 7,000 pg/ml to about 8,000 pg/ml, such as about 8,000 pg/ml to about 9,000 pg/ml, or such as about 9,000 pg/ml to about 10,000 pg/ml.

In certain embodiments, a biological factor can be secreted by a composition comprising a population of cells comprising OPCs at a concentration ranging from about 10,000 pg/ml to about 100,000 pg/ml, such as about 10,000 pg/ml to about 20,000 pg/ml, such as about 20,000 pg/ml to about 30,000 pg/ml, such as about 30,000 pg/ml to about 40,000 pg/ml, such as about 40,000 pg/ml to about 50,000 pg/ml, such as about 50,000 pg/ml to about 60,000 pg/ml, such as about 60,000 pg/ml to about 70,000 pg/ml, such as about 70,000 pg/ml to about 80,000 pg/ml, such as about 80,000 pg/ml to about 90,000 pg/ml, or such as about 90,000 pg/ml to about 100,000 pg/ml.

In some embodiments, Clusterin can be secreted by a composition comprising a population of cells comprising OPCs at a concentration ranging from about 1,000 pg/ml to about 100,000 pg/ml. In certain embodiments, Clusterin can be secreted by a composition comprising a population of cells comprising OPCs at a concentration ranging from about 10,000 pg/ml to about 50,000 pg/ml. In some embodiments, MCP-1 can be secreted by a composition comprising a population of cells comprising OPCs at a concentration ranging from about 500 pg/ml to about 50,000 pg/ml. In certain embodiments, MCP-1 can be secreted by a composition comprising a population of cells comprising OPCs at a concentration ranging from about 5,000 pg/ml to about 15,000 pg/ml. In some embodiments, ApoE can be secreted by a composition comprising a population of cells comprising OPCs at a concentration ranging from about 100 pg/ml to about 10,000 pg/ml. In certain embodiments, ApoE can be secreted by a composition comprising a population of cells comprising OPCs at a concentration ranging from about 500 pg/ml to about 5,000 pg/ml. In some embodiments, TIMP1 can be secreted by a composition comprising a population of cells comprising OPCs at a concentration ranging from about 100 pg/ml to about 10,000 pg/ml. In certain embodiments, TIMP1 can be secreted by a composition comprising a population of cells comprising OPCs at a concentration ranging from about 500 pg/ml to about 5,000 pg/ml. In some embodiments, TIMP2 can be secreted by a composition comprising a population of cells comprising OPCs at a concentration ranging from about 100 pg/ml to about 10,000 pg/ml. In certain embodiments, TIMP2 can be secreted by a composition comprising a population of cells comprising OPCs at a concentration ranging from about 500 pg/ml to about 5,000 pg/ml.

### Pharmaceutical Compositions

The OPCs of the present disclosure can be administered to a subject in need of therapy, such as SCI therapy. Alternatively, the cells of the present disclosure can be administered to the subject in need of SCI therapy in a pharmaceutical composition together with a suitable carrier and/or using a delivery system.

As used herein, the term “pharmaceutical composition” refers to a preparation comprising a therapeutic agent or therapeutic agents in combination with other components, such as physiologically suitable carriers and excipients.

As used herein, the term “therapeutic agent” can refer to the cells of the present disclosure accountable for a biological effect in the subject. Depending on the embodiment of the disclosure, “therapeutic agent” can refer to the oligodendrocyte progenitor cells of the disclosure. Alternatively, “therapeutic agent” can refer to one or more factors secreted by the oligodendrocyte progenitor cells of the disclosure.

As used herein, the terms “carrier”, “pharmaceutically acceptable carrier” and “biologically acceptable carrier” may be used interchangeably and refer to a diluent or a carrier substance that does not cause significant adverse effects or irritation in the subject and does not abrogate the biological activity or effect of the therapeutic agent. In certain embodiments, a pharmaceutically acceptable carrier can comprise dimethyl sulfoxide (DMSO). In other embodiments, a pharmaceutically acceptable carrier does not comprise dimethyl sulfoxide. The term “excipient” refers to an inert substance added to a pharmaceutical composition to further facilitate administration of the therapeutic agent.

The therapeutic agent or agents of the present disclosure can be administered as a component of a hydrogel, such as those described in U.S. patent application Ser. No. 14/275,795, filed May 12, 2014, and U.S. Pat. Nos. 8,324,184 and 7,928,069.

The compositions in accordance with the present disclosure can be formulated for parenteral administration by injection, e.g., by bolus injection or continuous infusion. Formulations for injection can be presented in unit dosage form, e.g., in ampoules or in multi-dose containers, with an added preservative. The compositions can contain formulatory agents such as suspending, stabilizing and/or dispersing agents. In certain embodiments, the compositions can be formulated to be adapted for cryopreservation.

The compositions in accordance with the present disclosure can be formulated for administration via injection to the spinal cord of a subject. The compositions may also be formulation for direct injection to the spinal cord of a subject. The compositions can be formulated for administration via implantation or other delivery methods. In certain embodiments, a composition in accordance with the present disclosure can be formulated for intracerebral, intraventricular, intrathecal, intranasal, or intracisternal administration to a subject. In certain embodiments, a composition in accordance with the present disclosure can be formulated for administration via an injection directly into or immediately adjacent to an infarct cavity in the brain of a subject. In certain embodiments, a composition in accordance with the present disclosure can be formulated for administration through implantation. In certain embodiments, a composition in accordance with the present disclosure can be formulated for administration through other suitable delivery methods. In certain embodiments, a composition in accordance with the present disclosure can be formulated as a solution.

In certain embodiments, a composition in accordance with the present disclosure can comprise from about 1×10⁶ to about 5×10⁸ cells per milliliter, such as about 1×10⁶ cells per milliliter, such as about 2×10⁶ cells per milliliter, such as about 3×10⁶ cells per milliliter, such as about 4×10⁶ cells per milliliter, such as about 5×10⁶ cells per milliliter, such as about 6×10⁶ cells per milliliter, such as about 7×10⁶ cells per milliliter, such as about 8×10⁶ cells per milliliter, such as about 9×10⁶ cells per milliliter, such as about 1×10⁷ cells per milliliter, such as about 2×10⁷ cells per milliliter, such as about 3×10⁷ cells per milliliter, such as about 4×10⁷ cells per milliliter, such as about 5×10⁷ cells per milliliter, such as about 6×10⁷ cells per milliliter, such as about 7×10⁷ cells per milliliter, such as about 8×10⁷ cells per milliliter, such as about 9×10⁷ cells per milliliter, such as about 1×10⁸ cells per milliliter, such as about 2×10⁸ cells per milliliter, such as about 3×10⁸ cells per milliliter, such as about 4×10⁸ cells per milliliter, or such as about 5×10⁸ cells per milliliter. In certain embodiments, a composition in accordance with the present disclosure can comprise from about 1×10⁸ to about 5×10⁸ cells per milliliter, such as about 1×10⁸ to about 4×10⁸ cells per milliliter, such as about 2×10⁸ to about 5×10⁸ cells per milliliter, such as about 1×10⁸ to about 3×10⁸ cells per milliliter, such as about 2×10⁸ to about 4×10⁸ cells per milliliter, or such as about 3×10⁸ to about 5×10⁸ cells per milliliter. In yet another embodiment, a composition in accordance with the present disclosure can comprise from about 1×10⁷ to about 1×10⁸ cells per milliliter, such as about 2×10⁷ to about 9×10⁷ cells per milliliter, such as about 3×10⁷ to about 8×10⁷ cells per milliliter, such as about 4×10⁷ to about 7×10⁷ cells per milliliter, or such as about 5×10⁷ to about 6×10⁷ cells per milliliter. In an embodiment, a composition in accordance with the present disclosure can comprise from about 1×10⁶ to about 1×10⁷ cells per milliliter, such as about 2×10⁶ to about 9×10⁶ cells per milliliter, such as about 3×10⁶ to about 8×10⁶ cells per milliliter, such as about 4×10⁶ to about 7×10⁶ cells per milliliter, or such as about 5×10⁶ to about 6×10⁶ cells per milliliter. In yet another embodiment, a composition in accordance with the present disclosure can comprise at least about 1×10⁶ cells per milliliter, such as at least about 2×10⁶ cells per milliliter, such as at least about 3×10⁶ cells per milliliter, such as at least about 4×10⁶ cells per milliliter, such as at least about 5×10⁶ cells per milliliter, such as at least about 6×10⁶ cells per milliliter, such as at least about 7×10⁶ cells per milliliter, such as at least about 8×10⁶ cells per milliliter, such as at least about 9×10⁶ cells per milliliter, such as at least about 1×10⁷ cells per milliliter, such as at least about 2×10⁷ cells per milliliter, such as at least about 3×10⁷ cells per milliliter, such as at least about 4×10⁷ cells per milliliter, or such as at least about 5×10⁷ cells per milliliter. In an embodiment, a composition in accordance with the present disclosure can comprise up to about 1×10⁸ cells or more, such as up to about 2×10⁸ cells per milliliter or more, such as up to about 3×10⁸ cells per milliliter or more, such as up to about 4×10⁸ cells per milliliter or more, such as up to about 5×10⁸ cells per milliliter or more, or such as up to about 6×10⁸ cells per milliliter.

In an embodiment, a composition in accordance with the present disclosure can comprise from about 4×10⁷ to about 2×10⁸ cells per milliliter.

In yet another embodiment, a composition in accordance with the present disclosure can have a volume ranging from about 10 microliters to about 5 milliliters, such as about 20 microliters, such as about 30 microliters, such as about 40 microliters, such as about 50 microliters, such as about 60 microliters, such as about 70 microliters, such as about 80 microliters, such as about 90 microliters, such as about 100 microliters, such as about 200 microliters, such as about 300 microliters, such as about 400 microliters, such as about 500 microliters, such as about 600 microliters, such as about 700 microliters, such as about 800 microliters, such as about 900 microliters, such as about 1 milliliter, such as about 1.5 milliliters, such as about 2 milliliters, such as about 2.5 milliliters, such as about 3 milliliters, such as about 3.5 milliliters, such as about 4 milliliters, or such as about 4.5 milliliters. In an embodiment, a composition in accordance with the present disclosure can have a volume ranging from about 10 microliters to about 100 microliters, such as about 20 microliters to about 90 microliters, such as about 30 microliters to about 80 microliters, such as about 40 microliters to about 70 microliters, or such as about 50 microliters to about 60 microliters. In another embodiment, a composition in accordance with the present disclosure can have a volume ranging from about 100 microliters to about 1 milliliter, such as about 200 microliters to about 900 microliters, such as about 300 microliters to about 800 microliters, such as about 400 microliters to about 700 microliters, or such as about 500 microliters to about 600 microliters. In yet another embodiment, a composition in accordance with the present disclosure can have a volume ranging from about 1 milliliter to about 5 milliliters, such as about 2 milliliter to about 5 milliliters, such as about 1 milliliter to about 4 milliliters, such as about 1 milliliter to about 3 milliliters, such as about 2 milliliter to about 4 milliliters, or such as about 3 milliliter to about 5 milliliters. In an embodiment, a composition in accordance with the present disclosure can have a volume of about 20 microliters to about 500 microliters. In another embodiment, a composition in accordance with the present disclosure can have a volume of about 50 microliters to about 100 microliters. In yet another embodiment, a composition in accordance with the present disclosure can have a volume of about 50 microliters to about 200 microliters. In another embodiment, a composition in accordance with the present disclosure can have a volume of about 20 microliters to about 400 microliters.

In certain embodiments, the present disclosure provides a container comprising a composition comprising a population of OPCs derived in accordance with one or more methods of the present disclosure. In certain embodiments, a container can be configured for cryopreservation. In certain embodiments, a container can be configured for administration to a subject in need thereof. In certain embodiments, a container can be a prefilled syringe.

For general principles in medicinal formulation, the reader is referred to Allogeneic Stem Cell Transplantation, Lazarus and Laughlin Eds. Springer Science+Business Media LLC 2010; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. In certain embodiments, the composition can also comprise or be accompanied by one or more other ingredients that facilitate the engraftment or functional mobilization of the enriched target cells. Suitable ingredients can include matrix proteins that support or promote adhesion of the target cell type or that promote vascularization of the implanted tissue.

### Uses of the Cells of the Present Disclosure

In various embodiments as described herein, the present disclosure provides methods of using a cell population that comprises pluripotent stem cell-derived OPCs for improving one or more neurological functions in a subject in need of therapy. In certain embodiments, methods for using pluripotent stem-cell derived OPCs in the treatment of acute spinal cord injury are provided. In other embodiments, methods for using pluripotent stem-cell derived OPCs in the treatment of other traumatic CNS injuries are provided. In other embodiments, methods for using pluripotent stem-cell derived OPCs in the treatment of non-traumatic CNS disorders or conditions are provided. In certain embodiments, a cell population in accordance with the present disclosure can be injected or implanted into a subject in need thereof.

In certain embodiments, methods for using pluripotent stem-cell derived OPCs in the treatment of conditions requiring myelin repair or remyelination are provided. The following are non-limiting examples of conditions, diseases and pathologies requiring myelin repair or remyelination: multiple sclerosis, the leukodystrophies, the Guillain-Barre Syndrome, the Charcot-Marie-Tooth neuropathy, Tay-Sachs disease, Niemann-Pick disease, Gaucher disease and Hurler syndrome. Other conditions that result in demyelination include but are not limited to inflammation, stroke, immune disorders, metabolic disorders and nutritional deficiencies (such as lack of vitamin B12). The OPCs of the present disclosure can also be used for myelin repair or remyelination in traumatic injuries resulting in loss of myelination, such as acute spinal cord injury.

The OPCs are administered in a manner that permits them to graft or migrate to the intended tissue site and reconstitute or regenerate the functionally deficient area. Administration of the cells can be achieved by any method known in the art. For example the cells can be administered surgically directly to the organ or tissue in need of a cellular transplant. Alternatively non-invasive procedures can be used to administer the cells to the subject. Non-limiting examples of non-invasive delivery methods include the use of syringes and/or catheters to deliver the cells into the organ or tissue in need of cellular therapy.

The subject receiving the OPCs of the present disclosure may be treated to reduce immune rejection of the transplanted cells. Methods contemplated include the administration of traditional immunosuppressive drugs such as, e.g., tacrolimus, cyclosporin A (Dunn et al., Drugs 61:1957, 2001), or inducing immunotolerance using a matched population of pluripotent stem cell-derived cells (WO 02/44343; U.S. Pat. No. 6,280,718; WO 03/050251). Alternatively a combination of anti-inflammatory (such as prednisone) and immunosuppressive drugs can be used. The OPCs of the invention can be supplied in the form of a pharmaceutical composition, comprising an isotonic excipient prepared under sufficiently sterile conditions for human administration.

Use in treatment of CNS traumatic injury. In certain embodiments, a cell population in accordance with the present disclosure can be capable of engrafting at a spinal cord injury site following implantation of a composition comprising the cell population into the spinal cord injury site.

In certain embodiments, a cell population in accordance with the present disclosure is capable of remaining within the spinal cord injury site of the subject for a period of about 180 days or longer following implantation of a dose of the composition into the spinal cord injury site. In other embodiments, a cell population in accordance with the present disclosure is capable of remaining within the spinal cord injury site of the subject for a period of about 2 years or longer following implantation of a dose of the composition into the spinal cord injury site. In further embodiments, a cell population in accordance with the present disclosure is capable of remaining within the spinal cord injury site of the subject for a period of about 3 years or longer following implantation of a dose of the composition into the spinal cord injury site. In yet further embodiments, a cell population in accordance with the present disclosure is capable of remaining within the spinal cord injury site of the subject for a period of about 4 years or longer following implantation of a dose of the composition into the spinal cord injury site.

In certain embodiments, a cell composition in accordance with the present disclosure is capable of reducing spinal cord injury-induced parenchymal cavitation in a subject. In certain embodiments, a lesion volume is reduced by formation of a tissue matrix in the spinal cord injury site. In certain embodiments, the cells of the present disclosure are capable of forming a tissue matrix in the spinal cord injury site within about 180 days or less. In certain embodiments, the subject with reduced injury-induced parenchymal cavitation is human.

In certain embodiments, a cell population in accordance with the present disclosure can be capable of reducing a volume of an injury-induced central nervous system parenchymal cavitation in about 12 months or less. In certain embodiments, a cell population in accordance with the present disclosure can be capable of reducing a volume of an injury-induced central nervous system parenchymal cavitation in a subject in about 6 months or less, about 5 months or less, or less than about 4 months. In certain embodiments, the subject is human.

In an embodiment, one or more cells from a cell population in accordance with the present disclosure can be capable of migrating from a first location to one or more second locations within the central nervous system of a subject in need thereof. In an embodiment, one or more cells from a cell population in accordance with the present disclosure can be capable of migrating from the spinal cord of a subject to an affected tissue within the brain of the subject. In one embodiment, one or more cells from a cell population in accordance with the present disclosure can be capable of migrating from a first location within the spinal cord of a subject to a second location at an affected tissue within the spinal cord of the subject. In one embodiment, one or more cells from a cell population in accordance with the present disclosure can be capable of migrating from a first location within the brain of a subject to a second location at an affected tissue within the brain of the subject. In one embodiment, one or more cells from a cell population in accordance with the present disclosure can be capable of migrating from a first location within the brain of a subject to an affected tissue within the spinal cord of the subject. In one embodiment, one or more cells from a cell population in accordance with the present disclosure can be capable of migrating from a first location within the spinal cord of a subject to a second location at an affected tissue within the spinal cord of the subject, as well as to one or more locations at one or more affected tissues within the brain of the subject. In one embodiment, one or more cells from a cell population in accordance with the present disclosure can be capable of migrating from a first location within the brain of a subject to a second location at an affected tissue within the brain of the subject, as well as to one or more locations at one or more affected tissues within the spinal cord of the subject.

In an embodiment, one or more cells from a cell population in accordance with the present disclosure can be capable of migrating from a first location to one or more second locations at one or more affected tissues within the central nervous system of a subject in less than about 150 days, such as less than about 100 days, such as less than about 50 days, or such as less than about 10 days. In an embodiment, one or more cells from a cell population in accordance with the present disclosure can be capable of migrating from a first location to one or more second locations at one or more affected tissues within the central nervous system of a subject in about 180 days or less.

### Parenchymal Spinal Delivery (PSD) System

A PSD system, including a PSD device, using repulsive forces produced by micromagnets to create a spring effect that compensates for spinal cord pulsation during intraspinal injections can be used to administer the composition as described herein. Is embodiments, the PSD device is a device as shown and described in PCT Patent Pub. No. WO2014047540, which is incorporated by reference in its entirety for all devices, systems, methods, and the like described therein.

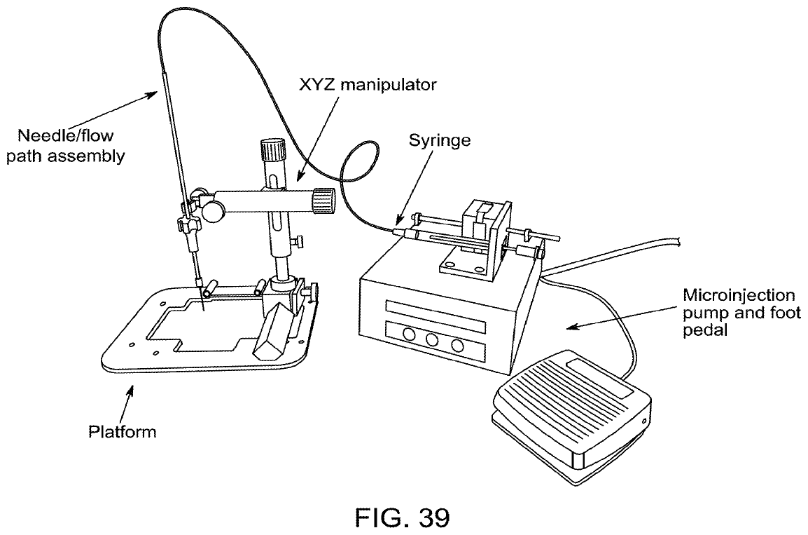

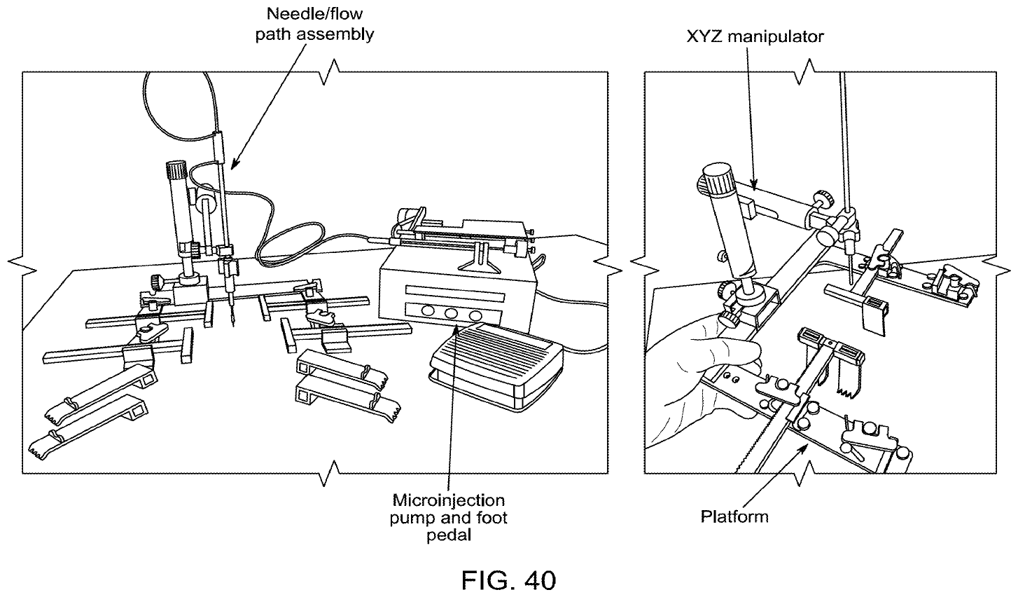

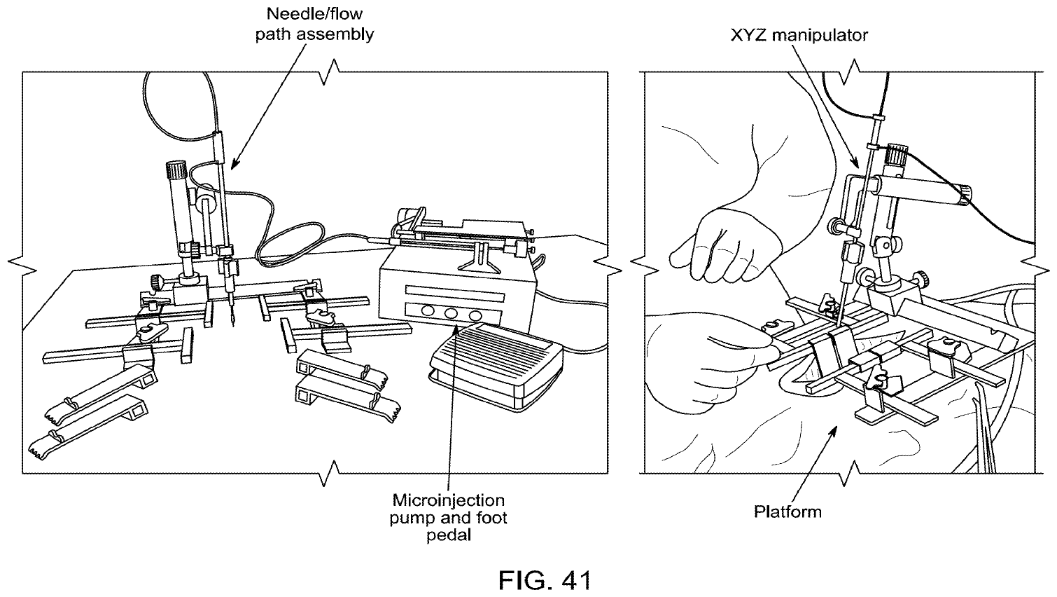

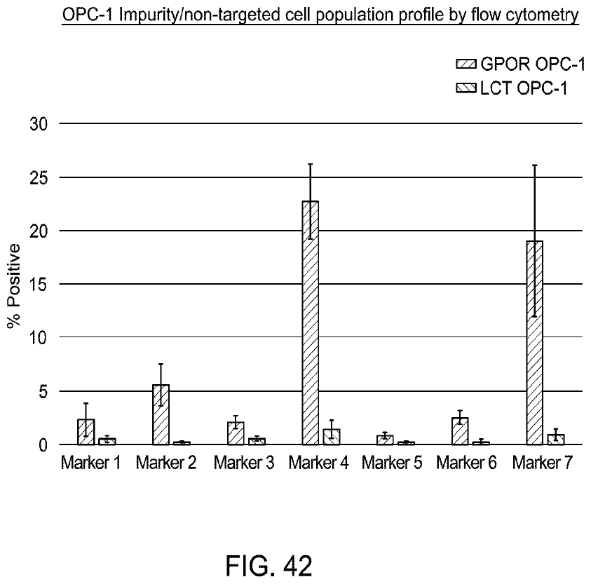

In one aspect, the PSD is as shown in any of FIGS. 39-41. In embodiments, the PSD is as shown in any of FIGS. 39-41.

In one aspect, the PSD device includes a frame having an elongated body and a plurality of holders extending therefrom; a plurality of first magnets, each being fixedly attached to a holder; a tube having a first end and a second end, the tube being slidingly disposed within through-holes disposed in each holder and in each first magnet; a plurality of second magnets fixedly attached to an exterior surface of the tube; and a needle fixedly attached to the first end of the tube. In embodiments, the frame may be made from any non-corrosive metal, such as stainless steel. In embodiments, the needle may range from about 27 to about 32 gauge. In embodiments, each of the first magnets and the each of the second magnets may be disposed such that a north pole of one first magnet faces a north pole of one second magnet or a south pole of one first magnet faces a south pole of one second magnet, thereby providing a magnetic repulsive force upon which the tube floats.

In various embodiments, the frame comprises two holders, each having attached thereto a first magnet, and a single second magnet is fixedly attached to the tube. In other embodiments, the frame comprises two holders, each having attached thereto a first magnet, and two second magnets are fixedly attached to the tube.

The device may further include a stop ring fixedly attached to the first end of the tube or an area near the first end of the tube. The device may further include a stop ring fixedly attached to the second end of the tube or an area near the second end of the tube. The device may further include tubing removably attached to the second end of the tube and configured for supplying a composition, e.g. the composition described herein, to the needle.

In embodiments, a needle stop ring may be fixedly attached to the needle to serve a guide to a surgeon as to the maximum distance that the needle will be lowered into the spinal cord during the procedure. When present, the stop ring may be positioned along the length of the needle such that when the needle reaches a predetermined depth, the stop ring contacts the subject/patient.

In embodiments, the stopper is located at about 1 mm to about 10 mm up the needle. In embodiments, the stopper is located at about 1 mm to about 8 mm up the needle (e.g., from the sharp end of the needle). In embodiments, the stopper is located about 4 mm to about 8 mm up the needle. In embodiments, the stopper is located at about 6 mm up the needle. The location of the stopper prevents the needle from traveling further into the spinal cord. The stopper allows for the ‘floating’ effect which compensates for spinal cord puslations caused by ventilation. This is a great advantage over other systems, as the patient does not need to be removed from the ventilator during injection.

In an embodiment, the system comprises a XYZ manipulator for manipulation of the position of a needle. In an embodiment, the device comprises a microinjection pump connected to a needle via a flow path (e.g., tubing) for delivery of the composition to the needle. In embodiments, the system comprises a foot pedal for operation of the pump. In an embodiment, the

In another aspect, the PSD system includes the PSD device described herein and a reservoir in fluid communication with the needle, the reservoir containing a composition to be administered to a subject. The system may further include a digital microinjector configured to control flow of the composition through the needle.

In various embodiments, the frame comprises two holders, each having attached thereto a first magnet, and a single second magnet is fixedly attached to the tube. In other embodiments, the frame comprises two holders, each having attached thereto a first magnet, and two second magnets are fixedly attached to the tube.

In yet another aspect, there is provided a method of compensating for spinal cord pulsation during administration of a composition to a spinal cord of a subject. The method includes positioning the PSD system over the spinal cord of the subject; lowering the needle into the spinal cord; and delivering a dose of the composition to the spinal cord. In embodiments, the needle and tube of the device float due to magnetic repulsive forces within the device, thereby compensating for spinal cord pulsation. In various embodiments, the method further includes repeating each of the steps at multiple sites along the spinal cord. The step of delivering may include activating a digital microinjector configured to control flow of the composition through the needle. The step of lowering the needle may include inserting the needle into the spinal parenchyma until a needle stop ring that is fixedly attached to the needle contacts the subject. Accordingly, the present invention also provides use of the PSD system described herein to compensate for spinal cord pulsation during administration of a composition as described herein.

The new PSD system device has several benefits. The device offers stability and control because it eliminates motion between platform/XYZ manipulator/injection needle. The pump and syringe are also not in a sterile field and allow for an accurate programmed dose rate. The device also requires no cessation of ventilation. It attaches directly to the patient and is compatible with patient breathing motion. The magnetic needle provides stabilization from micromotion due to heartbeats. The device is easier to use in a clinical setting because it is smaller and uses fewer components, it is easily assembled prior to surgery, it allows for single hand operation for XYZ positioning, accurate needle depth insertion, straightforward cleaning and sterilization, and it is compatible with OPC1 TAI formulation. Finally, it also eliminates the need for a prior-day dose preparation.

### EXAMPLES

Examples 1-8 describe the first-in-human Phase 1 safety clinical trial of oligodendrocyte progenitor cells derived from human pluripotent stem cells (LCTOPC1) which have mechanistic properties to support survival and potential repair of key cellular components and architecture of the SCI site. Example 9 describes a Phase 1/2a dose escalation study of oligodendrocyte progenitor cells derived from human pluripotent stem cells (AST-OPC1) for use in subacute cervical SCI.

### Example 1—Patients and Methods

Study design. The trial design was an open-label, multicenter study. A single dose of 2×10\hat{ }6 LCTOPC1 was injected within 7 to 14 days following SCI. Subjects who received LCTOPC1 also received tacrolimus to prevent rejection. Subjects will be followed by protocol for 15 years following administration of LCTOPC1.

Study Participants. Male or female participants from 18 to 65 years of age with acute traumatic spinal cord injury were eligible for study participation. As this was a first in man study, with a risk of neurological deterioration, inclusion was limited to neurologically complete injuries (American Spinal Injury Association Impairment Scale A), with a single neurological level of injury (NLI) from levels T3-T10, with no spared motor function <5 levels (i.e. zone of partial preservation) below the single neurological level. These inclusion criteria were chosen to minimize loss of function if neurological deterioration were to occur.

Post-stabilization magnetic resonance imaging (MRI) was used to confirm the presence of a single spinal cord lesion with sufficient visualization of the spinal cord for 30 mm above and below the injury epicenter to enable post-injection safety monitoring. Participants had to be eligible for an elective surgical procedure to inject LCTOPC1 7 to 14 days following SCI.

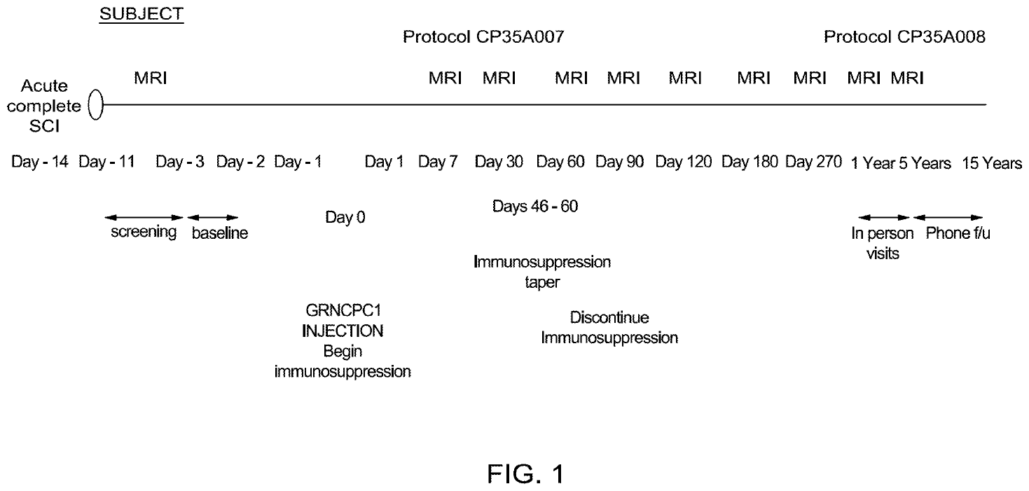

This study was a Phase 1, multi-center, non-randomized, a single group assignment interventional clinical trial. The Participants were enrolled from one of seven centers in the United Sates. The study was registered (NCT01217008) and the primary endpoint was safety, as measured by the frequency and severity of adverse events related to LCTOPC1, the injection procedure used to administer LCTOPC1, and/or the concomitant immunosuppression administered. The secondary endpoint was neurological function as measured by sensory scores and lower extremity motor scores on ISNCSCI examinations. The eligibility criteria are summarized in Supplemental Table 1. Participants have been followed by protocol for a total of 5 years of in-person visits and are being followed for an additional 10 years of annual phone visits. FIG. 1 and the following two paragraphs provide an overall study schema for the clinical trial.

The objective of the thoracic study was to evaluate the safety of oligodendrocyte progenitor cells derived from human pluripotent stem cells (LCTOPC1) administered between 7 and 14 days post injury to individuals with T3 to T11 neurologically complete spinal cord injuries (SCI). The rationale for this first-in-human trial was based on evidence that LCTOPC1 supports survival and potential repair of key cellular components and architecture of the SCI site. It was a multi-site, open-label, single-arm interventional clinical trial with an n=5.

The methods consisted of a single intra-parenchymal injection of 2×10⁶ OPC1 cells caudal to the epicenter of injury using a syringe positioning device. Immunosuppression with tacrolimus was administered for a total of 60 days. Follow-up consisted of annual in-person examinations and magnetic resonance imaging (MRI) for 5 years post injection, as well as annual phone questionnaires 6 to 15 years post injection. The primary endpoint was safety measured by the frequency and severity of adverse events related to the OPC1 injection, the injection procedure, and/or the concomitant immunosuppression administered. The secondary endpoint was neurological function as measured by sensory scores and lower extremity motor scores as measured by the ISNCSCI examinations.

The LCTOPC1 product is a cell population containing a mixture of oligodendrocyte progenitor cells and other characterized cell types obtained following directed differentiation of undifferentiated human embryonic stem cells. The initial characterization of the LCTOPC1 population was reported by Nistor et al 2005, who showed that these cells could differentiate into oligodendroglial progenitors. Subsequent studies demonstrated that the oligodendroglial progenitor cells survived after delivery to the spinal cord injury site in an acute incomplete rat contusion injury model. The cells led to sparing of tissue at the contusion site with evidence of remyelination of denuded axons. When delivered in the acute injury period, the cells led to improvement in locomotor function as measured in standardized behavioral testing. Preclinical studies in rats and mice demonstrated that the intended clinical, cryopreserved human equivalent dose formulation of LCTOPC1 could survive and migrate after injection in the SCI site, produce neurotrophic factors to support cell survival, provide remyelination potential to support denuded axons, and lead to tissue sparing at the SCI contusion site. Moreover, studies demonstrated that the cells did not produce teratomas, and did not lead to increased pain in injured animals.

This Phase 1 clinical trial was reviewed by the FDA, the Data and Safety Monitoring Board (DSMB), the SCI clinical community, surgical and outcomes steering committees, internal and external ethics committees, internal and clinical trial site stem cell research oversight committees, and the IRBs for each participating clinical trial site. As a first-in-human study, the trial design accounted for the need to minimize the risk to participants, and hence individuals with complete SCI localized between the thoracic neurological levels T3-T11 were chosen for intervention. The trial was an open-label, unblinded, non-randomized, non-placebo-controlled study to establish the safety of intraparenchymal injection of LCTPOC1 as well as to determine changes in neurological function.

Determining the long-term safety of stem cell therapeutics is a critical step in enabling future trials to investigate novel stem cell therapeutics or combination therapies. Ten years post-implantation, there have been no medical or neurological complications to indicate that LCTOPC1 implantation is unsafe. Specifically, there have been no Serious Adverse Events (SAEs) related to the procedure, cell implant, or immunosuppression. In addition, there have been no significant changes in neurological function. Safety data from this first-in-human study supported progression to a clinical trial for individuals with cervical spinal cord injuries.

The starting material for the production of AST-OPC1 is an H1 master cell bank produced from the H1 uhESC line derived at the University of Wisconsin in 1998. Compositional analysis of LCTOPC1 by immunocytochemistry and flow cytometry indicates that the cell population is comprised mostly of neural lineage cells of the oligodendrocyte progenitor phenotype. In this safety study, the intended route of administration for LCTOPC1 was a direct injection of 2×10\hat{ }6 viable LCTOPC1 cells into the spinal cord at a level 5 mm caudal to the injury epicenter.

The rationale for the selection of this dose was based upon pre-clinical studies involving rats and mice, and upon dose extrapolation to humans using two methods: 1) comparing the relative sizes of the human and rat spinal cords, and 2) evaluating tumorigenicity with respect to the absolute number of injected cells. Further, the rationale for the selection of this route of delivery was based on the results from nonclinical pharmacology studies, which showed that LCTOPC1 has properties that support repair of pathology in spinal cord lesions. Results from these studies also demonstrated that improvements in locomotor recovery were associated with robust LCTOPC1 cell survival at the lesion site.

Spinal cord injuries localized between the T3 and T11 neurological level, as assessed by the ISNCSCI, were chosen as the target for intervention. The primary goal of this first-in-human study was to establish the safety of intraparenchymal injection of hESC derived oligodendrocyte precursor cells into the spinal cord of individuals between 7-14 days post-injury. The secondary outcome measure in this trial was the ISNCSCI examination which allowed for the identification of motor and sensory changes at any of 13 in-person evaluations scheduled within the first five years post-injection.

Ten-years post-implantation, there have been no medical or neurological complications to indicate that the cell implantation was unsafe. Specifically, there have been no serious adverse events (SAEs) related to the procedure, cell implant, or immunosuppression. This report will review the first 10 years of data from this landmark clinical trial including early post-operative events, in-person follow-up through year 5 and conclude with data from telephone follow-up to the current time.

Investigational Product, Dose Preparation, Dose and Mode of Administration. LCTOPC1 is a cell population containing a mixture of oligodendrocyte progenitor cells and other characterized cell types that are obtained following differentiation of undifferentiated human embryonic stem cells (hESC) from the H1 stem cell line, produced at the University of Wisconsin in 1998.

Compositional analysis of LCTOPC1 by immunocytochemistry and flow cytometry indicates that the cell population is comprised mostly of neural lineage cells of the oligodendrocyte progenitor phenotype. In this safety study, the intended route of administration for LCTOPC1 was a direct injection of 2×10\hat{ }6 viable LCTOPC1 cells into the spinal cord at a level 5 mm caudal to the injury epicenter.

LCTOPC1 is a cryopreserved cell therapy product. At the time of cryopreservation, each vial contained 7.5×10⁶ viable cells in 1.2 mL of cryoprotectant solution. LCTOPC1 was supplied in 2.0 mL cryovials and shipped to the clinical sites in the vapor phase of liquid nitrogen and stored under the same conditions at the site. Prior to administration, LCTOPC1 was thawed and prepared by study personnel who were trained and qualified in the preparation of the study drug.

Participants received a single administration of 2×10⁶ viable LCTOPC1 cells suspended in Hank's balanced salt solution (HBSS) total volume per dose=50 μL. The rationale for selection of this dose was based upon pre-clinical studies involving rats and mice, and upon dose extrapolation to humans using two methods: 1) comparing the relative sizes of the human and rat spinal cords, and 2) evaluating tumorigenicity risks with respect to the absolute number of injected cells. At that time during the development of LCTOPC1, 2×10⁶ cells was the highest dose that was feasible to administer in the injured rat spinal cord and the rat was the largest animal that could be utilized to satisfy the animal number required for the IND-enabling studies for this novel product. Hence, to be conservative, 2×10⁶ cells, the highest dose tested in rats, was used as the dose for the Phase 1 trial. Participants who received LCTOPC1 also received tacrolimus to prevent rejection of this allogeneic cell-based product.

The intended route of administration for LCTOPC1 was intra-parenchymal at a depth of 6 mm, in the midline, 5 mm caudal to the epicenter of injury as determined by MRI, as modeled in preclinical studies. A caudal injection was selected out of an abundance of caution to avoid any potential direct tissue damage above the injury level. Based on preclinical studies, it was anticipated that the injected cells would migrate rostrally throughout the injury site. LCTOPC1 was administered to the spinal cord in a dedicated surgical procedure 7-14 days following injury. This time frame was chosen based on results of animal studies suggesting poor graft survival for implantation within the first 7 days of injury while attempting to maximize the potential neuroprotective and remyelinating effect. A custom-designed syringe positioning device was utilized to assist neurosurgeons with the controlled delivery of the cells. In other examples, the route of administration can include administering to the subject the composition (e.g., including LCTOPC1) using a parenchymal spinal delivery (PSD) system, for example a PSD system as described herein. In embodiments, the composition is administered using a PSD system as shown and described in U.S. Publication No: 2015/0224331, incorporated by reference herein in its entirety.

Tacrolimus Immunosuppression. Immunosuppression with tacrolimus was initiated between 6 and 12 hours after injection of LCTOPC1. If the participant was unable to take oral medication, tacrolimus was administered intravenously at a starting dose of 0.01 mg/kg/day, given as a continuous intravenous infusion. Participants were switched to oral tacrolimus as possible. The starting dose for oral tacrolimus was 0.03 mg/kg/day, divided into 2 daily doses. The tacrolimus dose was adjusted to achieve a target whole blood trough level of 3 to 7 ng/ml.

On Day 46, the tacrolimus dose was decreased by 50% (rounded to the nearest 0.5 mg, as this was the smallest capsule size available). On Day 53, the tacrolimus dose was decreased by another 50% (rounded to the nearest 0.5 mg). If the rounded total daily dose was 0.5 mg or lower, the participant received 0.5 mg once per day until tacrolimus was discontinued. Tacrolimus was discontinued at Day 60. The dose of tacrolimus was lowered if the trough blood level exceeded 7 ng/mL. In addition, an expert reviewed all ISNCSCI examination forms to assess whether there were any changes in neurological function that may have been associated with tacrolimus tapering and/or discontinuation.

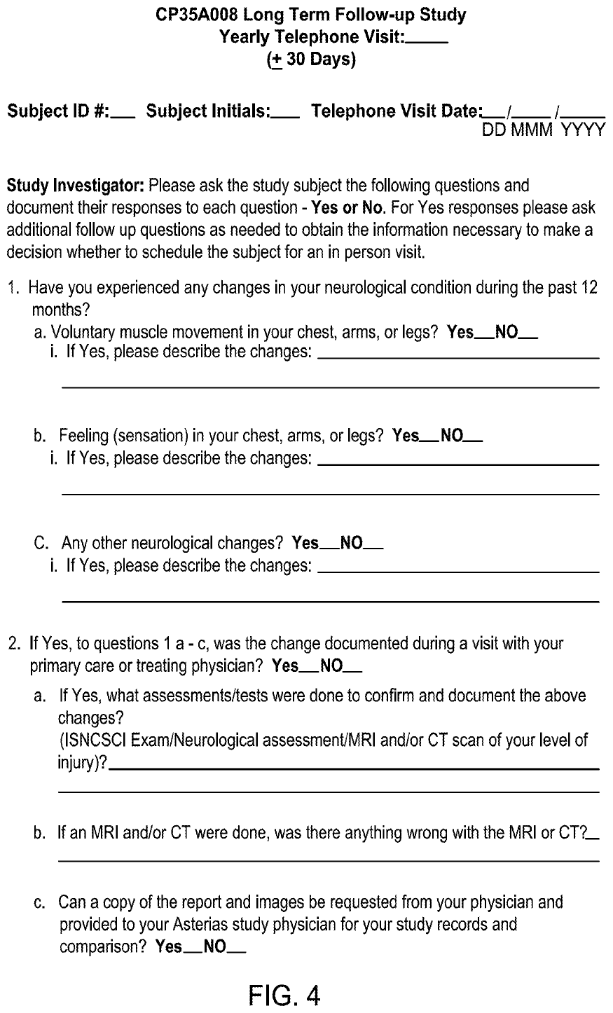

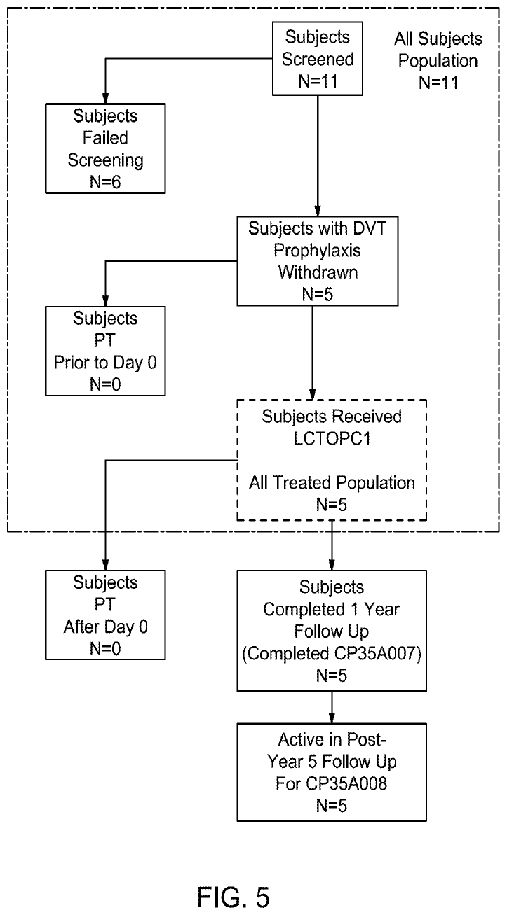

Follow-up and Assessments. An overview of study visits for the one-year protocol follow-up (CP35A007) and 2-15-year protocol follow-up (CP35A008) is provided in the study schema (FIG. 1). As this was a first clinical trial of cells derived from hESCs, a high number of study visits and long-term follow-up were required. In the one-year protocol, three study visits were required prior to product administration, with 13 in the first year following study administration. For the long-term protocol, annual visits were required in years 2-5. Subsequent to the year 5 annual visit, follow-up was by annual phone questionnaires (FIG. 4) and in-person evaluations, as necessary. Phone assessments include documentation of all new medications taken for longer than 30 days, admissions to the hospital, and documentation of AEs and SAEs.

Safety Assessments. The primary endpoint of the Phase 1 clinical trial was safety, as measured by the frequency and severity of adverse events (AEs) within 1 year of LCTOPC1 injection that were related to LCTOPC1, the injection procedure used to administer LCTOPC1, and/or the concomitant immunosuppression administered. Safety assessments included physical examination, vital signs, ISNCSCI neurological examination, pain questionnaire, electrocardiogram, MRI, laboratory tests for hematology and blood chemistry, laboratory tests for immunosuppression safety monitoring (whole blood trough levels of tacrolimus, serum levels of creatinine, potassium, magnesium, phosphate, ionized calcium, aspartate aminotransferase, alanine aminotransferase, and total bilirubin), concomitant medications, and AEs.

Definition of an Adverse Event. AEs were tabulated by system organ class and by preferred term within system organ class according to the Medical Dictionary for Regulatory Activities (MedDRAR) Version 10. An AE was any untoward medical event that occurred to a study participant once the participant had signed the informed consent form until the study participant's last study visit, whether or not the event was considered drug-related. The severity of AEs and the characterization of Serious Adverse Events (SAEs) were evaluated using standard FDA criteria.

The relationship of AEs to the investigational drug was determined by each site investigator and was categorized as “Possibly Related” based on the following criteria: 1) the AE was reasonably related in time with LCTOPC1 exposure, the injection procedure used to administer LCTOPC1, and/or the concomitant immunosuppression administered AND 2) the AE could be explained either by exposure to the investigational product or equally well by factors or causes other than exposure to the investigational product. Adverse events were monitored by the External Medical Monitor, Sponsor Medical Monitor, and DSMB.

Neurological Assessments. The secondary endpoint was neurological function including measurement of sensory scores and lower extremity motor scores. Neurological function was evaluated using the ISNCSCI examination for motor and sensory testing and for designation of the American Spinal Injury Association (ASIA) impairment scale (AIS).

Exploratory Endpoints. Pain assessment was performed using the International Spinal Cord Injury Pain Basic Data Set. A set of three questions was added to assess allodynia. These questions covered the presence and severity of pain provoked or increased by brushing, pressure or contact with cold. Information on pain medication was collected as part of the assessment of concomitant medications. Potential exploratory endpoints for recovery of neurological function were: University of Alabama-Birmingham Index of Motor Recovery (UAB-IMR), Spinal Cord Independence Measure (SCIM), and assessment of bowel and bladder function.

Lumbar Puncture. A lumbar puncture to obtain 10 mL of cerebrospinal fluid (CSF) was conducted after receiving general anesthesia but prior to LCTOPC1 injection as well as at day 60 post-injection. The volume required at individual study sites for the following tests were sent to the hospital laboratory: white blood cell count, glucose, total protein, oligoclonal banding, myelin basic protein, and immunoglobulin G index. In addition, CSF was evaluated by the sponsor to assess immune response to LCTOPC1.

Magnetic Resonance Imaging. Screening/Baseline MRI was obtained between 3 and 5 days prior to injection (Day-3 and Day-5) of LCTOPC1 but no earlier than 4 days after SCI. Screening/baseline MRI included the brain, cerebellum, and entire spinal cord, with and without contrast (gadolinium dietheylenetriamine pentaacetic acid [Gd-DTPA]). If surgery for LCTOPC1 injection was subsequently delayed for more than 3 days, then a repeat MRI of the thoracic spine, without contrast, was obtained. Follow-up MRIs of the spinal cord and cerebellum, with and without contrast (Gd-DTPA), were obtained on Days 7, 60, 120, and 270 post-injection. A full central nervous system MRI, with and without contrast (Gd-DTPA), was obtained on Days 30, 90, 180, and 365 as well as yearly between years 2-5. Image acquisition protocols were standardized. Image review was centralized and standardized with by an independent radiologist, DD at Radiology Imaging Associates Denver.

HLA Typing and Immunological Monitoring. LCTOPC1 cells do not express Human Leukocyte Antigen (HLA) Class II and are resistant to NK cell lysis. However, one concern in regard to the safety and potential efficacy of LCTOPC1 was the possibility of allogeneic rejection by the host's immune system. Immunosuppression was minimized in terms of duration to 60 days and dosed below the typical long-term maintenance therapy levels used for solid organ transplant. Peripheral blood and cerebrospinal fluid (CSF) samples from LSTOPC1 injected participants were collected according to protocol. A lumbar puncture to obtain 10 mL of CSF was conducted after receiving general anesthesia but prior to LCTOPC1 injection as well as at day 60 post-injection to assess for rejection of allogenic cells as well as for immunologic monitoring. The following assessments occurred at the hospital laboratory: white blood cell count, glucose, total protein, oligoclonal banding, myelin basic protein, and immunoglobulin G index. Peripheral blood was examined for the presence of antibodies specific for the donor-specific HLA molecules on LCTOPC1 and to detect T cell-mediated responses to LCTOPC1. In addition, CSF was evaluated by the sponsor to further assess immune response to LCTOPC1 and for the presence of LCTOPC1 (day 60) using a PCR based assay.

Statistical Methods. Descriptive analysis was used due to the small sample size, open-label, and non-randomized study design. The primary and secondary endpoints of this study are presented descriptively in table, figure, and text form.

### Results

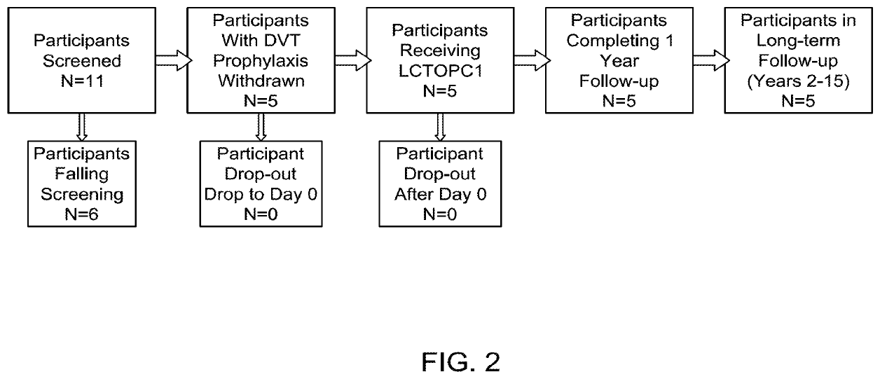

Study Participants. The first participant was implanted the winter of 2010 and the last participant was enrolled in the winter of 2011. Eleven individuals with SCI were screened for enrollment, with six individuals who failed screening: four due to MRI artifacts which prohibited adequate spinal cord visualization, one based on the ISNCSCI examination (NLI T1), and one due to elevated liver enzymes. A total of five individuals with SCI received LCTOPC1 at three study sites. FIG. 2 provides a Consolidated Standard of Reporting Trials (CONSORT) flow diagram. In this trial, the most common mechanism of injury was motor vehicle-related for four of five individuals, with a fall being the cause of injury in one individual. Four of five participants enrolled were male. The cohort age ranged from 21 to 32 years of age (Table 1).

TABLE 1
Demographic and Baseline Disease Characteristics - All Treated Subjects.

| | Age | | | Weight | BMI | |
|---|---|---|---|---|---|---|
| Subject | (years) | Sex | Race | (kg) | (kg/m²) | Cause of Injury |
| 1002 | 21 | Male | Caucasian | 63.1 | 21.1 | Motor vehicle accident |
| 1003 | 23 | Male | Caucasian | 78.4 | 23.4 | Restrained driver in rollover motor |
| | | | | | | vehicle collision with ejection |
| 1101 | 32 | Male | Caucasian | 70.5 | 22.9 | Motocross |
| 1203 | 31 | Male | Caucasian | 82.7 | 27.7 | Fell 30 feet down rock embankment |
| 1204 | 23 | Female | Caucasian | 61.5 | 24.8 | Car accident |

Abbreviations: BMI = body mass index, kg = kilograms, m = meter.

Participant Follow-up. As of this publication, all participants have entered their tenth year of follow-up. In agreement with the FDA, the trial was structured to begin with 5 years of in-person evaluation followed in years 6 through 15 with phone interviews. During the first 5 years of the study, 24 of 25 in-person annual visits were completed. One participant did not participate in the year 5 in-person visit but has participated in scheduled phone follow-up. From year 6 to the current time, 21 of 21 annual phone interviews have been completed. One participant has completed 10 years of follow-up and four participants are entering their 10-year follow-up interviews.

Primary Outcome Measure: Evaluation of Safety. All SAEs and AEs (related and unrelated to procedure, cell implant, or immunosuppression) are summarized in Table 2 and described below.

TABLE 2

| System Organ Class (SOC) | Total (N = 5) | |
|---|---|---|
| Preferred Term | Number of Events | n (%) |
| All events | 172 | |
| Nervous system disorders | 19 | 4 (80.0) |
| Eye disorders | 2 | 2 (40.0) |
| Gastrointestinal disorders | 16 | 5 (100) |
| General disorders and administration site | 8 | 3 (60.0) |
| conditions | | |
| Immune system disorders | 2 | 2 (40.0) |
| Infections and infestations | 42 | 5 (100) |
| Injury, poisoning and procedural | 10 | 5 (100) |
| complications | | |
| Investigations | 5 | 3 (60.0) |
| Metabolism and nutrition disorders | 3 | 2 (40.0) |
| Musculoskeletal and connective tissue | 32 | 5 (100) |
| disorders | | |
| Psychiatric disorders | 8 | 2 (40.0) |
| Renal and urinary disorders | 7 | 4 (80.0) |
| Reproductive system and breast disorders | 1 | 1 (20.0) |
| Respiratory, thoracic and mediastinal | 2 | 2 (40.0) |
| disorders | | |
| Skin and subcutaneous tissue disorders | 11 | 3 (60.0) |
| Surgical and medical procedures | 2 | 2 (40.0) |
| Vascular disorders | 2 | 1 (20.0) |

N = number of subjects in safety population or number of subjects with respective event category;
n = number of subjects in respective category;
% = n * 100/N.

Serious Adverse Events Related to Procedure, Cell Implant, or Immunosuppression. There were no SAEs related to the procedure, cell implant, or immunosuppression. There were no findings of clinical concern on MRI scans of the full central nervous system through five years post-injection in any participant. During long-term phone follow-up participants denied having any fever of unknown cause, no changes in sensation in chest, arms, or legs (other than described below), and no participants have been diagnosed with any type of cancer. No participants died during the study. Safety events were monitored by the DSMB and no suspension rules were triggered.

Serious Adverse Events Unrelated to Procedure, Cell Implant, or Immunosuppression. Three participants have reported four SAEs unrelated to the procedure, cell implant, or immunosuppression. These SAEs included urinary tract infection (UTI) and subsequent transitory autonomic dysreflexia in one individual, pyelonephritis, and a mood disorder in two different individuals.

Adverse Events Categorized by Grade. Over the course of the trial, 25 AEs were judged by the Investigators to be possibly related to LCTOPC1 (Grade 1/Mild [n=9], Grade 2/Moderate [n=15], and Grade 3/Severe [n=1]). The Grade 3 AE was described as a burning sensation in the trunk and lower extremities first reported on Day 57 post-injection with Grade 1 severity, increasing to Grade 3 severity on Day 90 post-injection. This neuropathic pain resulted in three additional Grade 2 severity AEs and was ongoing through year 9 follow-up. Grade 2 AEs included: surgical site pain, hypomagnesemia, urinary tract infection, vaginal yeast infection, emesis, upper back pain, shoulder pain, pain with range of motion, and autonomic discomfort during catheterization relieved after treatment with lidocaine. Grade 1 AEs included: hypomagnesemia, urinary tract infection, fever, headache, nausea, and spasticity.

Adverse Events Categorized by Relation to Procedure, Cell Implant, or Immunosuppression. Nine of the 25 related adverse events were deemed possibly related specifically to the injection procedure. Eight of the nine were Grade 1 or 2 in severity and one was Grade 3. The AEs were predominantly transient postoperative pain, one fever, and one urinary tract infection. There were no AEs attributed to the cell implant. Moreover, the immunosuppression regiment was well tolerated, and all five participants completed the regimen per protocol. Sixteen of the 25 adverse events were deemed possibly related specifically to the immunosuppression. Seven Grade 1 AEs and nine Grade 2 AEs were judged to be possibly related specifically to tacrolimus. These AEs were primarily known common adverse reactions to tacrolimus (nausea/emesis, low magnesium level, infections). Among reported infections, 1 of 7 was a vaginal yeast infection and 6 of 7 infections were in the urinary tract, which is a common complication of SCI.

Adverse Events Unrelated to Procedure, Cell Implant, or Immunosuppression. At year 6, one participant reported an increase in frequency and intensity of muscle spasms attributed to functional electrical stimulation (FES) cycling. This participant reported resolution of these symptoms during years 7 through 9 and is currently not using any medication for muscle spasms. In year 9, a different individual received outpatient testing after developing a deep vein thrombosis (DVT).

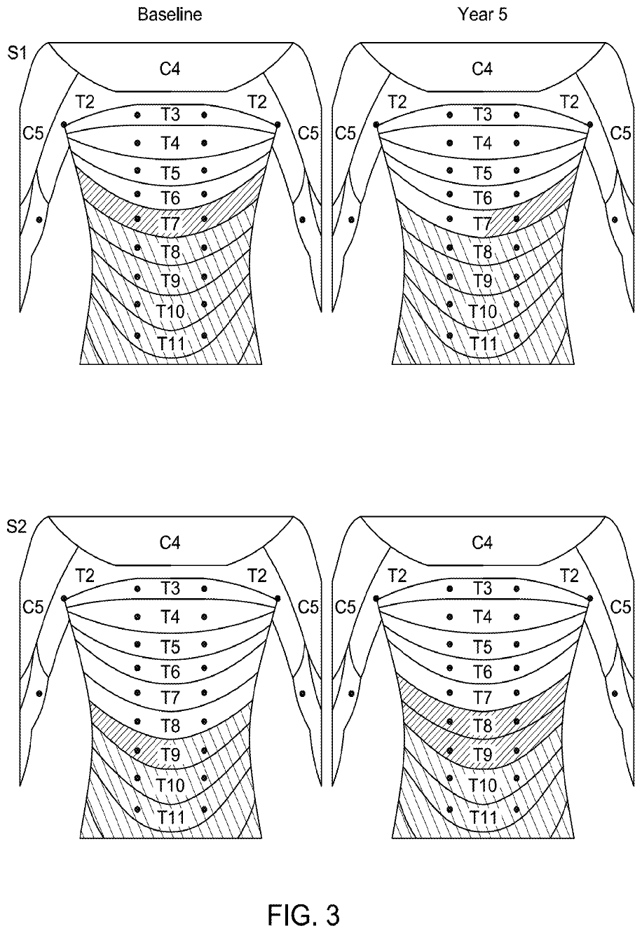

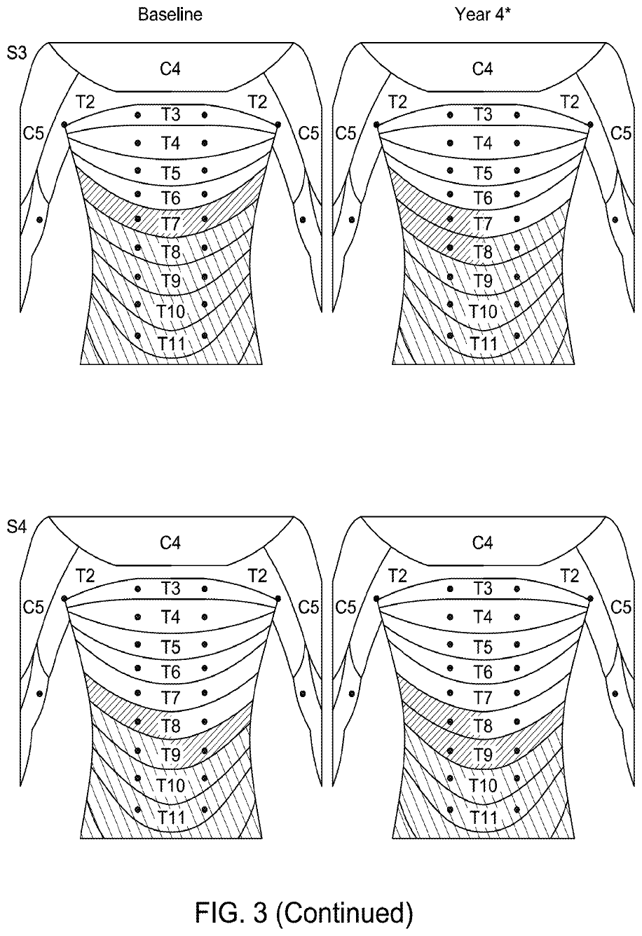

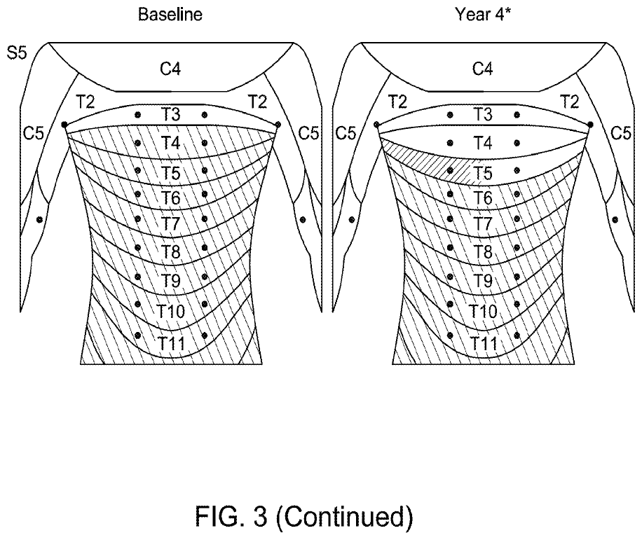

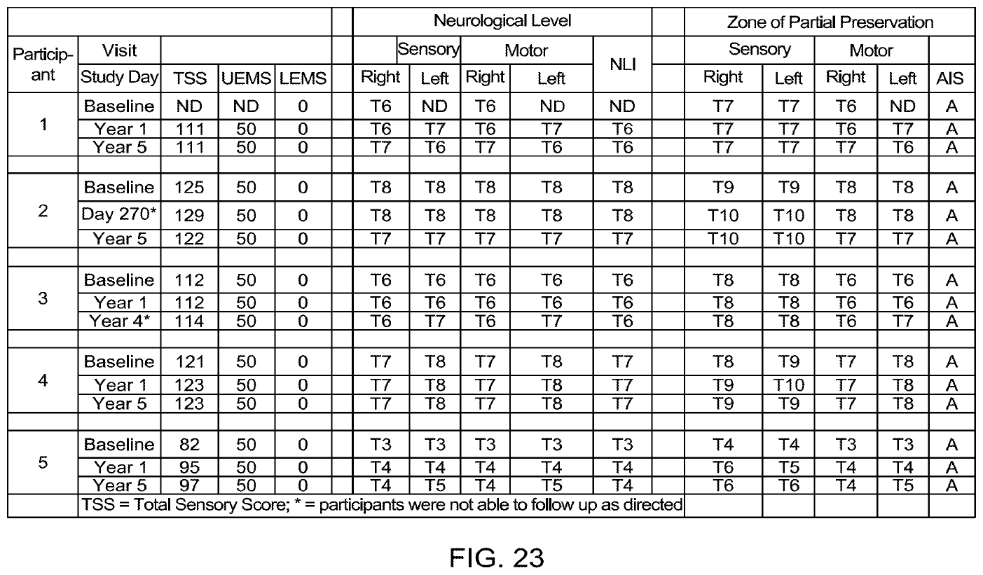

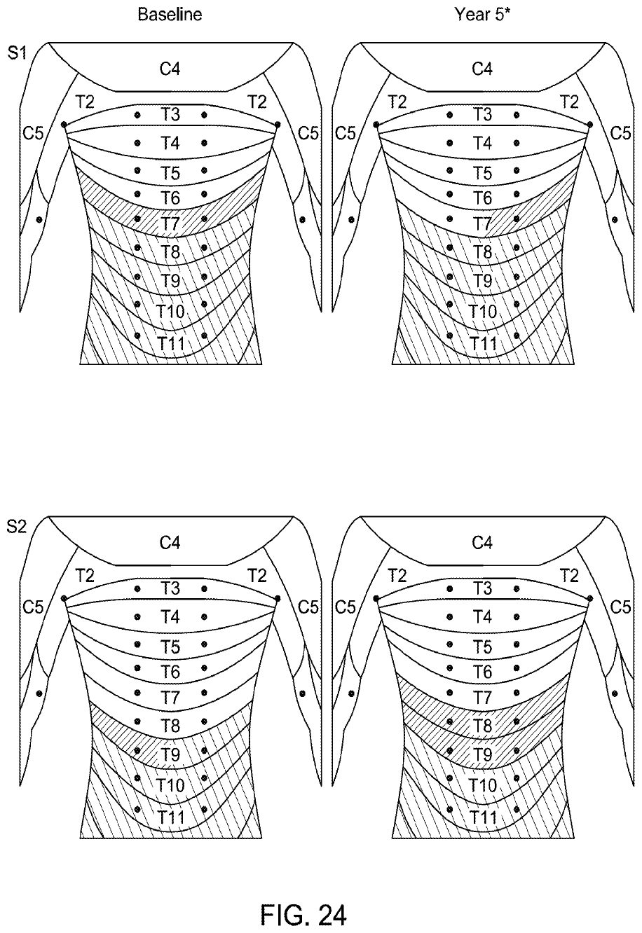

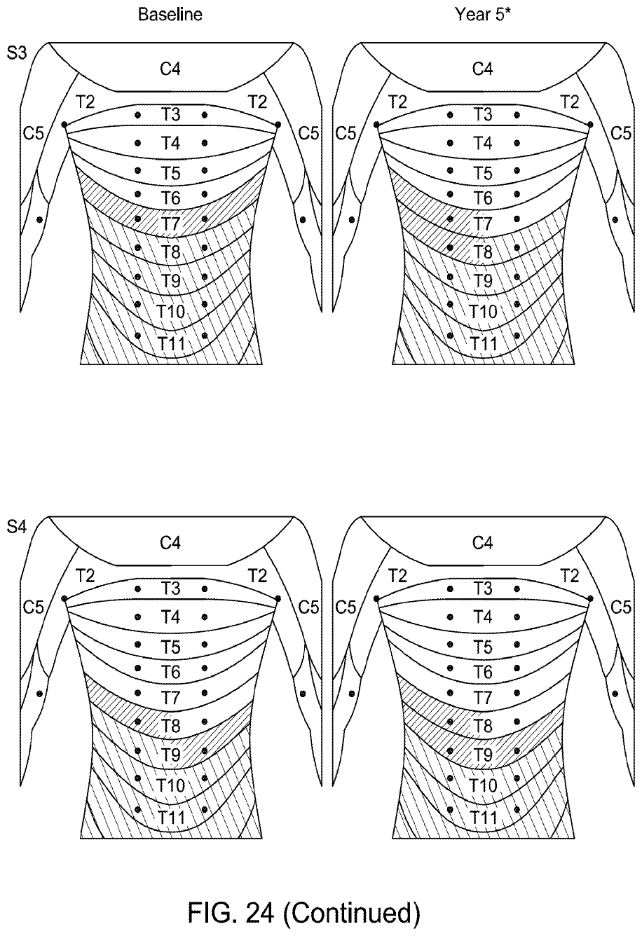

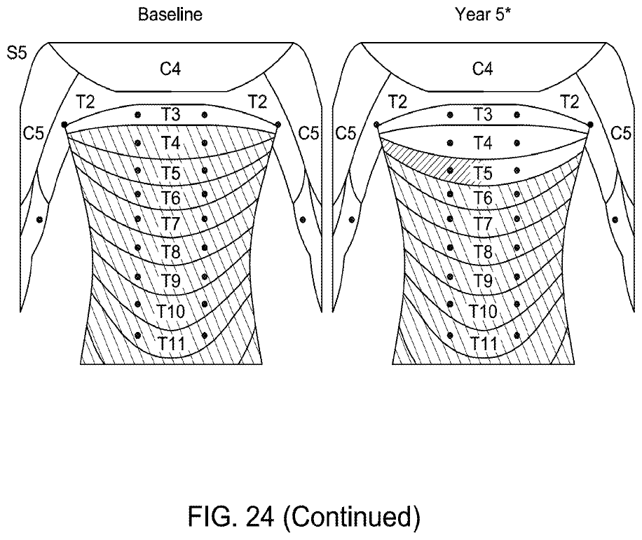

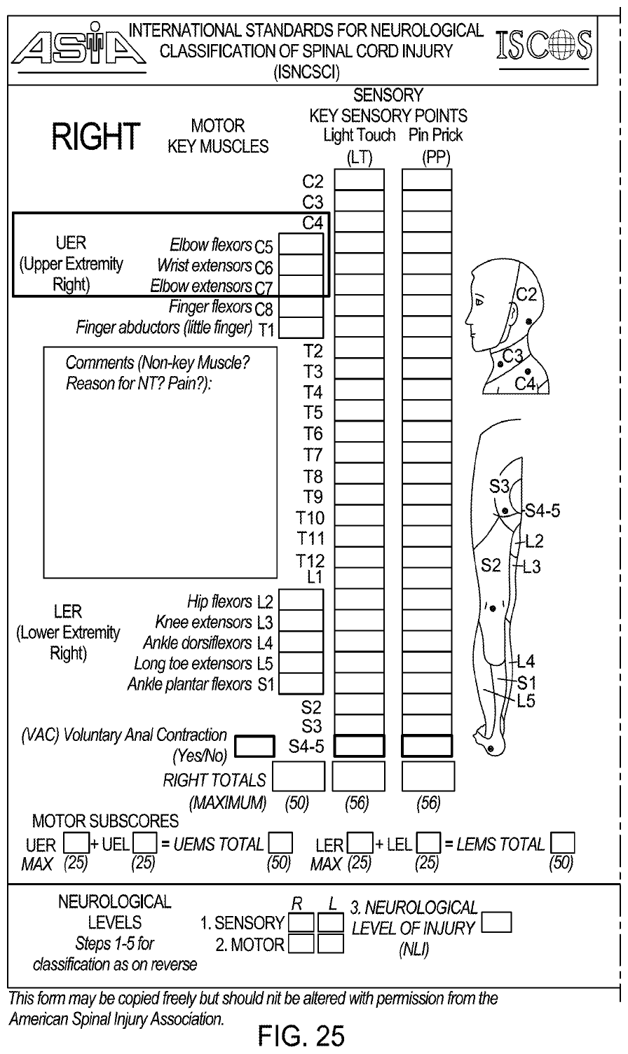

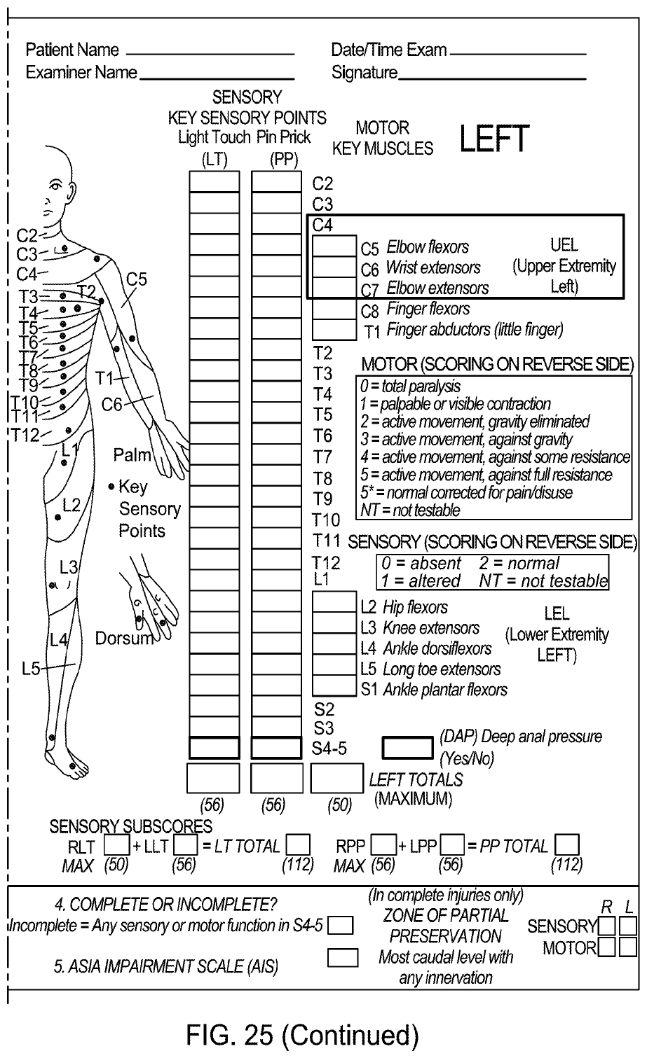

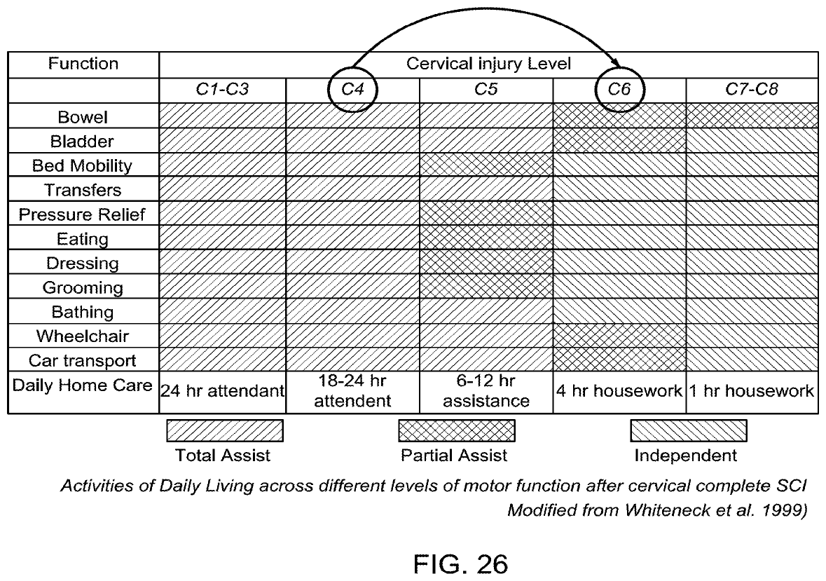

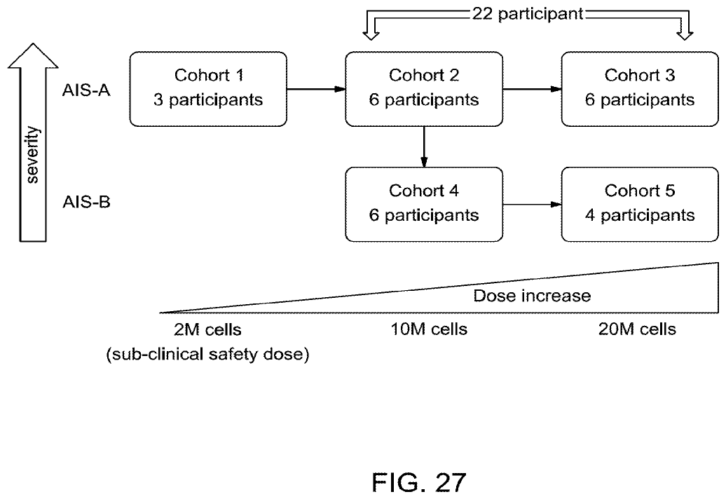

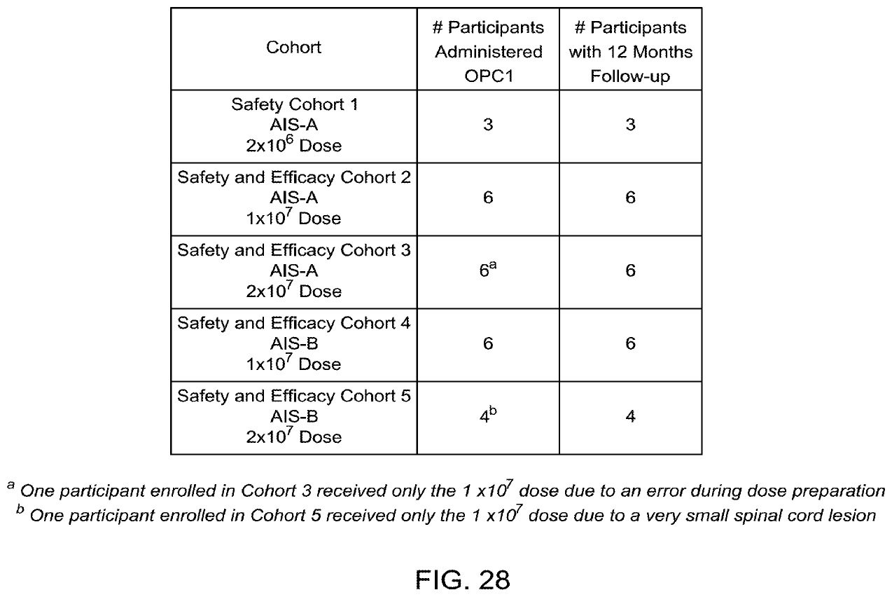

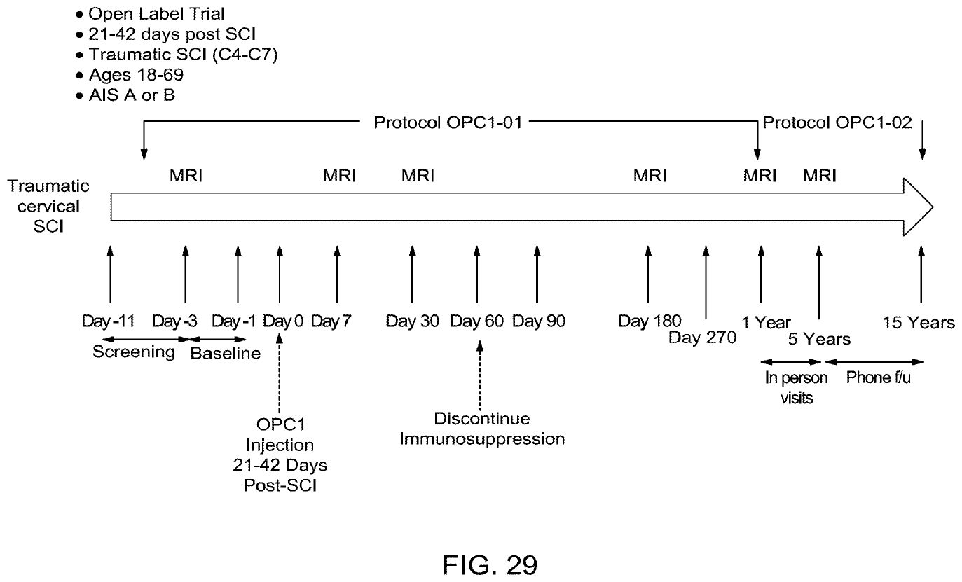

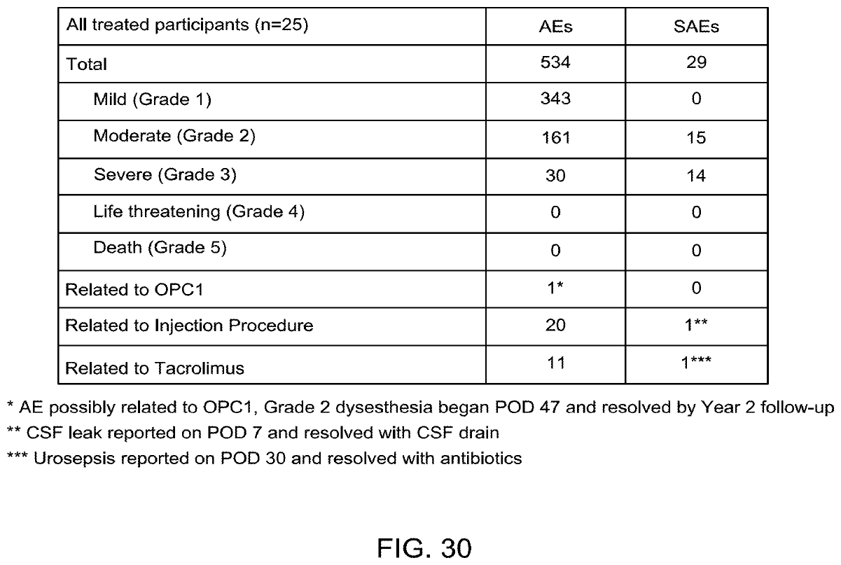

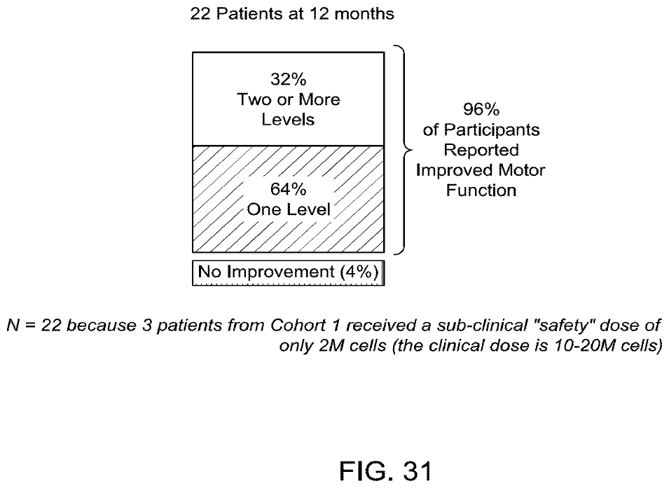

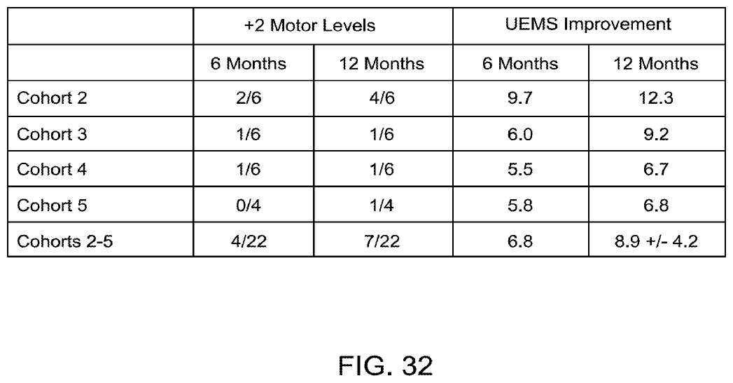

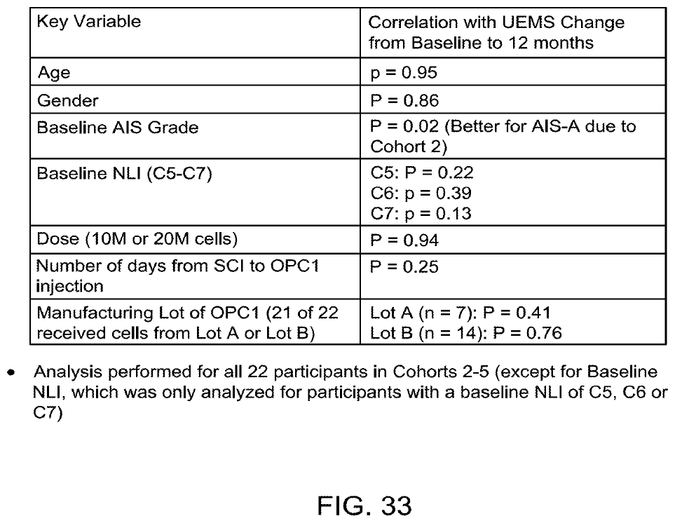

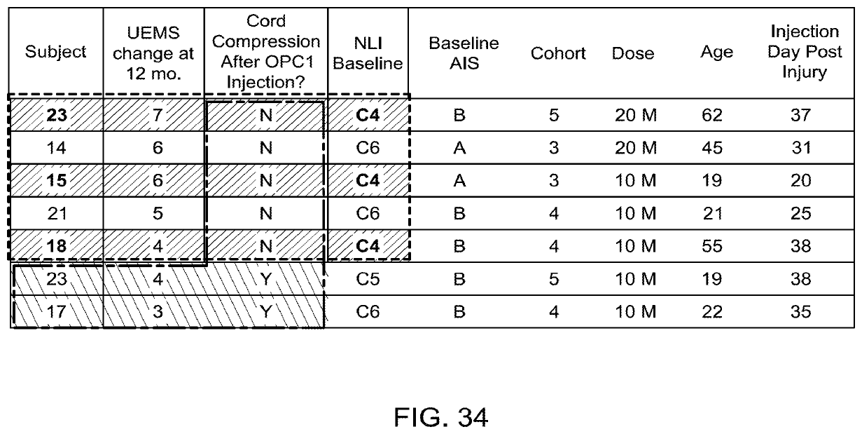

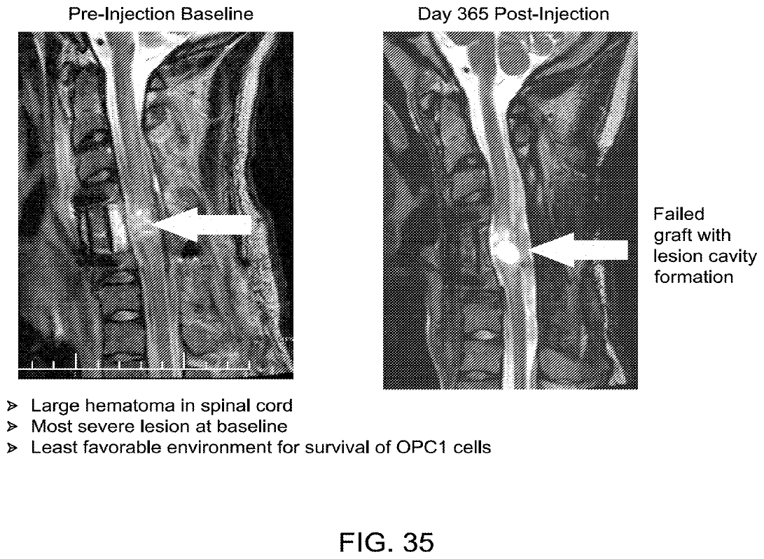

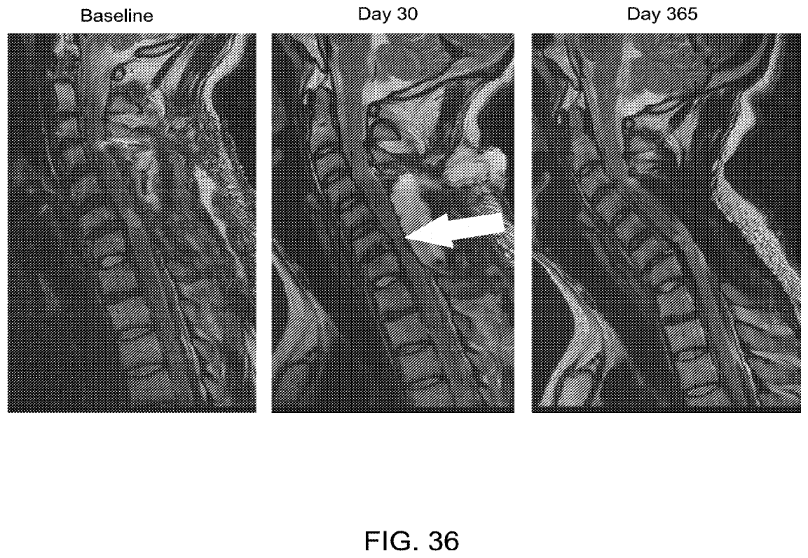

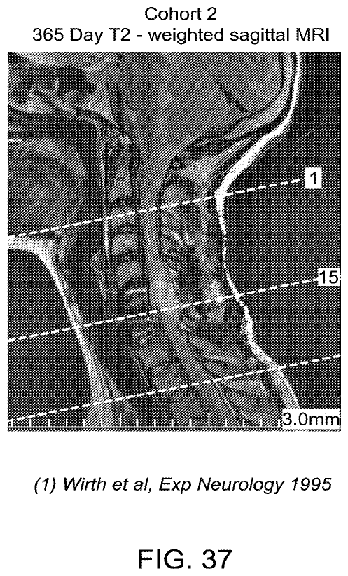

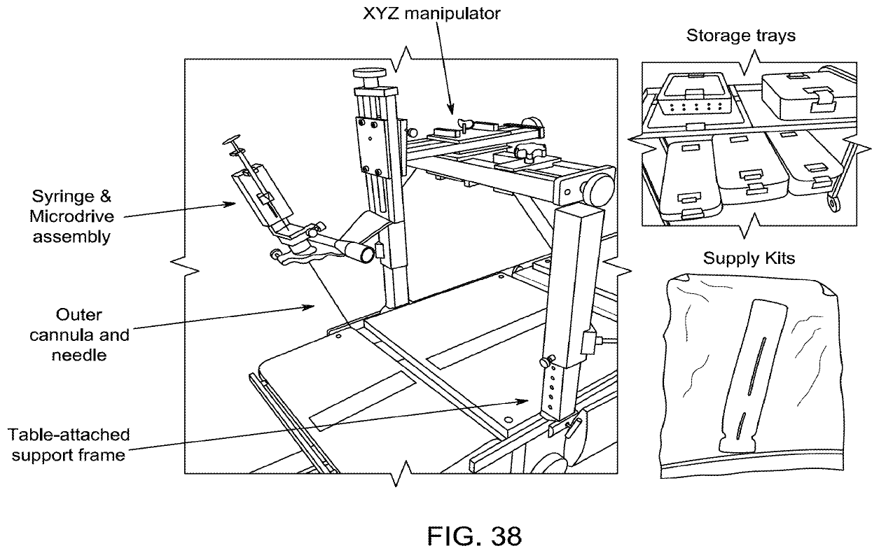

Secondary Outcome Measure: Neurological Assessment. After discharge from acute inpatient rehabilitation and through the first five years post-implantation, participants continued to be evaluated in-person according to the schedule shown in FIG. 1. Of note, between baseline and Year 5, participants' annual in-person evaluations included at least 13 ISNCSCI exams. All participants had an ASIA impairment Score (AIS) grade of A on enrollment in the trial and all participants have maintained the same impairment grade. The highest single and lowest NLI enrolled in the study were T3 and T8 respectively. Only the individual with T3 NLI improved to T4 with a sensory ZPP initially at T4 bilaterally noted to improve to T5 on the left and T6 on the right at one year follow-up. In total three of five participants experienced at least one level of improvement in their ZPP. All participants began and ended the 5 years of in-person ISNCSCI examination with intact upper extremity motor function with an upper extremity motor score (UEMS) of 50 out of 50, and lower extremity motor score (LEMS) of 0 out of 50 (Table 3). Over the course of 5 years of in-person follow-up, sensory examinations have not materially changed. FIGS. 3 and 23 provide a diagrammatic representation of the motor and sensory function of each patient at baseline and at 5 years post LCTOPC1 administration.

TABLE 3
ISNCSCI Baseline, Year 1 post-transplantation, and Year 5 post-transplantation.

| | | | | | | | | | | | Zone of Partial | | | | |
|---|---|---|---|---|---|---|---|---|---|---|---|---|---|---|---|
| | | | | | Neurologic Level | | | | | | Preservation | | | | |
| | Visit | | | | | Sensory | | Motor | | Single | Sensory | | Motor | | |
| Subject | Study | Date | TSS | UEM | LEMS | Right | Left | Right | Left | Score | Right | Left | Right | Left | AIS |
| | Baseline | 6 Oct. 2010 | ND | ND | 0 | T6 | ND | T6 | ND | ND | T7 | T7 | T6 | ND | A |
| 1002 | Year 1 | 6 Oct. 2011 | 111 | 50 | 0 | T6 | T7 | T6 | T7 | T6 | T7 | T7 | T6 | T7 | A |
| | Year 5 | 6 Oct. 2015 | 111 | 50 | 0 | T7 | T6 | T7 | T6 | T6 | T7 | T7 | T7 | T6 | A |
| | Baseline | 7 Aug. 2011 | 125 | 50 | 0 | T8 | T8 | T8 | T8 | T8 | T9 | T9 | T8 | T8 | A |
| 1003 | Day 270* | 3 May 2012 | 129 | 50 | 0 | T8 | T8 | T8 | T8 | T8 | T10 | T10 | T8 | T8 | A |
| | Year 5 | 2 Aug. 2016 | 122 | 50 | 0 | T7 | T7 | T7 | T7 | T7 | T10 | T10 | T7 | T7 | A |
| | Baseline | 5 May 2011 | 112 | 50 | 0 | T6 | T6 | T6 | T6 | T6 | T8 | T8 | T6 | T6 | A |
| 1101 | Year 1 | 24 Apr. 2012 | 112 | 50 | 0 | T6 | T6 | T6 | T6 | T6 | T8 | T8 | T6 | T6 | A |
| | Year 4* | 4 May 2015 | 114 | 50 | 0 | T6 | T7 | T6 | T7 | T6 | T8 | T8 | T6 | T7 | A |
| | Baseline | 15 Sept. 2011 | 121 | 50 | 0 | T7 | T8 | T7 | T8 | T7 | T8 | T9 | T7 | T8 | A |
| 1203 | Year 1 | 18 Sep. 2012 | 123 | 50 | 0 | T7 | T8 | T7 | T8 | T7 | T9 | T10 | T7 | T8 | A |
| | Year 5 | 17 Oct. 2016 | 123 | 50 | 0 | T7 | T8 | T7 | T8 | T7 | T9 | T9 | T7 | T8 | A |
| | Baseline | 14 Nov. 2011 | 82 | 50 | 0 | T3 | T3 | T3 | T3 | T3 | T4 | T4 | T3 | T3 | A |
| 1204 | Year 1 | 13 Nov. 2012 | 95 | 50 | 0 | T4 | T4 | T4 | T4 | T4 | T6 | T5 | T4 | T4 | A |
| | Year 5 | 31 Oct. 2016 | 97 | 50 | 0 | T4 | T5 | T4 | T5 | T4 | T6 | T6 | T4 | T6 | A |

TSS = Total Sensory Score; *= participants were not able to follow u

Table 3 represents total sensory score (TSS), upper extremity motor score (UEMS), lower extremity motor score (LEMS), sensory neurological level of injury (NLI), motor NLI, sensory zone of partial preservation (ZPP), Motor ZPP, and ASIA impairment Score (AIS) grade at baseline, year 1 and year 5. All five individuals were AIS Grade A at enrollment and there were no conversions to AIS B. The highest single and lowest NLI enrolled in the study were T3 and T8 respectively. Only the individual with the T3 NLI improved to T4 with a sensory ZPP initially at T4 bilaterally noted to improve to T5 on the left and T6 on the right at one year follow up. In total three of five participants experienced at least 1 level of improvement in their ZPP. ND=Unable to determine.

MRI Findings. No participant exhibited evidence of an enlarging cyst, enlarging mass, spinal cord damage related to the injection procedure, intramedullary hemorrhage, CSF leak, epidural abscess or infection, inflammatory lesions in the spinal cord, CSF flow obstruction, or masses in the ventricular system. No evidence of any adverse neurological changes or adverse changes on MRI was reported during tacrolimus tapering or after tacrolimus discontinuation.

Immune Monitoring. LSTOPC1 is an allogeneic cell therapy and is potentially sensitive to rejection by the recipient immune system. As a baseline assessment, HLA Class I and Class II molecular typing was performed for 10 alleles of the donor LCTOPC1 cells and peripheral blood cells of each of the 5 recipients. The potential development of a cellular immune response to LCTOPC1 was assessed and showed no evidence of T-cell mediated responses to LCTOPC1 even after cessation of tacrolimus immunosuppression in any of the serum samples of the five recipients. In addition, CSF samples obtained through lumbar puncture did not show signs of antibody or T-cell responses to LCTOPC1.

### Discussion

In January of 2009, Nature reported that LCTOPC1 would enter “the world's first clinical trial of a therapy generated by human embryonic stem cells”. At the time, pharmaceutical research in acute SCI was considered a relatively recent development. Although no clinical trial of hESC-derived cell lines had ever been assessed in any context, procedures for other intraparenchymal injections of cellular products (e.g. activated autologous macrophages) into the spinal cord had been evaluated providing a partial roadmap for LCTOPC1 based studies.

We present the primary and secondary outcome measures of five participants who received 2×10⁶ allogeneic hESC derived oligodendrocyte progenitor cells within 7-14 days post-injury. The primary results from the first 10 years of follow-up establish the safety and feasibility of intraparenchymal LCTOPC1 injection. The injection procedure and the low-dose immunosuppression regimen were well tolerated. To date, all five participants who received LCTOPC1 demonstrate no evidence of neurological deterioration or adverse findings on MRI scans.

This study was not designed to assess efficacy; however, animal studies of LCTOPC1 produced improvements in motor function through mechanisms that appeared to represent remyelination as well as neuroprotection, suppression of inflammation, promotion of axonal regeneration, and/or homeostatic maintenance. The proposed mechanism of locomotor function improvement included remyelination as well as expression of neurotrophic factors. The limited signs of functional recovery in various human trials despite promising results in animals may be related to the relative severity of human injuries in comparison to preclinical studies with incomplete contusion, suggesting that subsequent studies with incomplete injuries may demonstrate recovery more similar to that seen in animal models. Although this study did not demonstrate significant recovery, no participant exhibited evidence of neurological deterioration on ISNCSCI examinations through 5 years of in-person follow-up or 10 years of self-reported neurological function. The total motor score as well as the total sensory score of the ISNCSCI exam have remained stable across time. No unanticipated SAEs related to LCTOPC1 have been reported with 98% follow-up of participants (45 of 46 annual visits) through the first 10 years of the clinical trial.

Neuropathic pain in response to LCTOPC1 secondary to remyelination or neurotrophic factors was assessed using the International Spinal Cord Injury Pain Basic Data Set and a set of three questions to assess allodynia. Neuropathic at level pain and below level pain often have onset during the first several months after SCI, and by 1 year the prevalence of neuropathic pain approaches 60%. The prevalence of pain in this study is consistent with the natural history of neuropathic pain. One participant experienced neuropathic pain reported as a burning sensation in the trunk and lower extremities that was considered possibly related to LCTOPC1, which persisted in long-term follow up. The pain reported by this participant is consistent with two of the major categories of pain that are common following SCI: neuropathic pain at the level of injury (termed neuropathic at level pain), and neuropathic pain that occurs diffusely below the level of injury (termed neuropathic below level pain). Unfortunately for affected individuals, both at level and below level neuropathic pain are often severe and persistent for at least 5 years after SCI, despite attempts at pain management. In addition, 40 to 50% of individuals with these types of pain report their pain as severe or excruciating. It is not possible to determine a cause-and-effect relationship between LCTOPC1 or change in the incidence of long-term neuropathic pain in this small open-label study.

Serial MRI studies did not demonstrate the formation of ectopic tissue and/or teratomas. In addition to the absence of space occupying lesions, the natural history of chronic SCI MRI studies suggests that cavitary lesions will be identifiable in 58% of individuals who pursue thoracic level cellular trials. MRI results during the long-term follow-up period for LCTOPC1 were of particular significance because 80% of individuals showed T2 signal changes consistent with the formation of a tissue matrix at the injury site. Although the sample size is limited, these findings suggest that LCTOPC1 cells may have either durable engraftment and/or induced long-term changes which limited cavitation at the injury site.

SCI is a relatively rare condition and the potential population of T3-T11 AIS A injuries represent less than 20% of acute SCI in the United States (NSCISC National Spinal Cord Injury Statistical Center, University of Alabama at Birmingham, 2011 Annual Statistical Report-Complete Public Version).

### Conclusion

The LCTOPC1 thoracic SCI clinical trial is one of the longest running clinical trials in the hESC field. The study provides crucial first-in-human positive safety data for future hESC derived therapies. While we cannot exclude the possibility of future adverse events, the experience in this trial provides evidence that these treatments can be well tolerated and event-free for up to 10 years. In addition, this report supports the willingness of participants to participate in long-term follow-up as well as setting a standard for corporate sponsors' commitment to data collection beyond their immediate financial interests. Based on the safety profile of LCTOPC1 obtained in this study, a cervical dose escalation trial was initiated.

### Stem Cell Materials and Methods

The following Examples provide exemplary methods for obtaining cells that can be used in the methods and uses described herein. Additional methods, materials, and uses can be found, for example, in WO/2020/154533, which is incorporated herein by reference in its entirety.

### Example 2—Culture and Expansion of Undifferentiated Human Embryonic Stem Cells

Undifferentiated human embryonic stem cells (uhESC) from a working cell bank (WCB) generated from the H1 line (WA01; Thomson J A, Itskovitz-Eldor J, Shapiro S S, Waknitz M A, Swiergiel J J, Marshall V S, Jones J M. Embryonic stem cell lines derived from human blastocysts. Science. 1998 Nov. 6; 282 (5391): 1145-7) were cultured on recombinant human Laminin-521 (rhLn-521, Corning #354224) coated, tissue culture treated polystyrene T-75 culture flasks (Corning #431082) in complete mTeSRIM-1 medium (Stem Cell Technologies #85850). The medium was completely exchanged daily until the cells reached approximately 80-90% confluency, and uhESCs were then passaged using ReLeSR™ reagent (Stem Cell Technologies #05872). ReLeSR™-lifted uhESC cells were seeded in new rhLn-521 coated 225 cm² flasks, and daily medium exchange was resumed two days post-seeding. Cultured uhESCs from the WCB were expanded in this manner for between two to five passages, depending on the experiment, prior to differentiation into neuroectoderm progenitor cells as described in Example 3.

### Example 3—Method of Differentiating Human Embryonic Stem Cells to Neuroectoderm Progenitors with Dorsal Spinal Cord Progenitor Phenotype

Expanded uhESC were seeded on rhLn-521-coated vessels, and cultured until reaching 40-70% confluency at which point differentiation was initiated. Days 0-3: Differentiation was initiated by complete removal of mTeSR™-1 medium, and addition of Glial Progenitor Medium (GPM; consisting of DMEM/F12 (Gibco Catalog No. 10565-018) supplemented with 2% B27 supplement (Gibco Catalog No. 17504-044), and 0.04 μg/mL tri-iodo-thyronine (Sigma-Aldrich Catalog No. T5516-1 MG)) supplemented with 10 μM of MAPK/ERK inhibitor, PD0325901 (PD; Sigma-Aldrich Catalog No. PZ0162), 2 μM of BMP signaling inhibitor, Dorsomorphin (Dorso; Sigma-Aldrich Catalog No. P5499), and 1 μM of Retinoic Acid (RA; Sigma-Aldrich Catalog No. R2625). This medium was replenished daily. Days 4-6: On Day 4, culture medium was switched to GPM supplemented with 1 μM RA and 150 μM Ascorbic Acid (Sigma-Aldrich Catalog No. A4544) and replenished daily. Day 7: On Day 7, the cells were harvested for expansion and further differentiation into glial progenitors as described in Example 4. A subset of cells were collected for analysis by quantitative PCR (qPCR; as described in Example 7), flow cytometry (as described in Example 6), and when available, separate well plates set aside for analysis were prepared for immunocytochemistry (ICC) (as described in Example 6). At Day 7, these cells exhibited marker expression consistent with dorsal spinal cord progenitors (Table 4).

TABLE 4
qPCR analysis of gene markers for pluripotency and neuroectoderm
progenitor cells (NPCs) in H1 uhESCs differentiated into NPCs
using different combinations of small molecule inhibitors.

| | | | | | |
|---|---|---|---|---|---|
| PD0325901 + | AZD6244 + | GSK1120212 + | PD184352 + | Cobimetinib + | PD0325901 + |
| Dorso | Dorso | Dorso | Dorso | Dorso | ML347 |

Pluripotency genes

| | | | | | | |
|---|---|---|---|---|---|---|
| NANOG | 1 | 4 | 1 | 1 | 1 | 1 |
| LIN28A | 548 | 618 | 606 | 635 | 504 | 488 |
| SOX2 | 370 | 484 | 254 | 401 | 201 | 202 |

Neuroectoderm progenitor cell genes

| | | | | | | |
|---|---|---|---|---|---|---|
| PAX6 | 363 | 539 | 258 | 417 | 173 | 156 |
| HES5 | 97 | 100 | 68 | 91 | 36 | 36 |
| ZBTB16 | 135 | 149 | 145 | 171 | 134 | 120 |

Dorsal spinal cord progenitor cell genes

| | | | | | | |
|---|---|---|---|---|---|---|
| TFAP2A | 67 | 61 | 84 | 43 | 100 | 166 |
| PAX3 | 112 | 99 | 180 | 84 | 193 | 158 |
| PAX7 | 156 | 106 | 186 | 100 | 148 | 209 |

Ventral spinal cord progenitor cell genes

| | | | | | | |
|---|---|---|---|---|---|---|
| OLIG2 | 1 | 1 | 1 | 1 | 1 | 1 |
| NKX2-2 | 1 | 1 | 1 | 1 | 1 | 1 |

### Example 4—Method of Differentiating Human Embryonic Stem Cells to Glial Lineage Cells

Days 7-13: Differentiation of uhESCs to neuroectoderm progenitors, specifically of a dorsal phenotype, was performed as described in Example 3. On Day 7, the cells were lifted using TrypLE™ Select (Thermo Fisher, cat #A12859-01), counted, and seeded onto rhLn-521-coated vessels at a seeding density of 2.7×10+ cells/cm² in GPM supplemented with 20 ng/ml human basic fibroblast growth factor (hbFGF, Thermo Fisher, cat #PHG0263), 10 ng/ml epidermal growth factor (EGF, Thermo Fisher, cat #PHG0311), and 10 μM Rho Kinase Inhibitor (RI, Tocris Catalog No. 1254). Culture medium was replenished daily by aspirating spent medium and replacing it with fresh GPM+hbFGF+EGF. Days 14-21: At Day 14, cells were lifted using TrypLE™ Select, counted, resuspended in GPM+hbFGF+EGF+RI, and reseeded in dynamic suspension cultures at a density of 1.83×10⁶ viable cells/mL into either PBS-0.1 L or PBS-0.5 L Mini Bioreactor Systems (PBS Biotech). Subsets of cells at Day 14 were collected for analysis by flow cytometry (Example 6), ICC (Example 6), and qPCR (Example 7). PBS0.1 L and PBS0.5 L Mini Bioreactors were set to rotate at 35 RPM and 28 RPM, respectively. Culture medium was replenished daily by allowing the aggregates to settle, removing 70-80% of spent medium, and replacing with an equal volume of GPM+bhFGF+EGF. On Day 15, the rotation velocity was increased to 45 RPM and 32 RPM for the PBS0.1 L and PBS0.5 L Mini Bioreactors, respectively. At Day 21, subsets of the aggregates were collected for ICC (Example 6) and qPCR (Example 7). By Day 21, the differentiated cells expressed markers consistent with glial-restricted cells (Table 4).

### Example 5—Method of Differentiating Human Embryonic Stem Cells to Oligodendrocyte Progenitor Cells

Days 21-42: The glial-restricted progenitor cells obtained in Example 4 were further differentiated into oligodendrocyte progenitor cells (OPCs). The differentiation protocol for Days 0-20 was performed as described in Examples 3 and 4. On Day 21, aggregates were transferred from dynamic suspension to rhLn-521-coated culture vessels. For example, starting with 1×PBS-0.1 L Mini Bioreactor with 60 mL of total volume, the 60 mL of culture was split onto 2×T75 flasks, each with 30 mL of volume. Subsequently, cells were fed every other day with GPM supplemented with 20 ng/mL EGF and 10 ng/mL of platelet-derived growth factor AA (PDGFAA; PeproTech, cat #AF-100-13A). Every seven days, (i.e., Day 28 and Day 35), cells were lifted with TrypLE™ Select, counted, and reseeded onto fresh rhLn-521-coated culture vessels at a seeding density of 4×10⁴ viable cells/cm². The differentiated cells were harvested on Day 42. Cells were detached from vessels using TrypLEIM Select, counted, and re-formulated in CryoStor10 (BioLife Solutions, cat #210102) prior to cryopreservation. Subsets of cells were collected for analysis by flow cytometry (Example 6), ICC (Example 6), and qPCR (Example 7). By Day 42, the differentiated cells expressed markers characteristic with OPCs as measured by the three analytical methods (Table 4).

### Example 6—Characterization of Differentiated Cell Populations by Immunocytochemistry and Flow Cytometry

Flow cytometry and immunocytochemistry (ICC) can be used to detect and characterize different aspects of protein marker expression in a cell population. While flow cytometry can be used to quantify the percentage of individual cells within the population that exhibit a given protein marker profile, ICC provides additional information about the subcellular localization of each protein marker and can be applied to single cells or cellular aggregates. By using either or both of these protein profiling approaches, we tracked the differentiation of human embryonic stem cells to neuroectoderm progenitor cells, glial progenitor cells, and oligodendrocyte progenitor cells according to the methods of the present disclosure. For human embryonic stem cells differentiated into neuroectoderm progenitor cells and glial progenitor cells, protein marker expression in the differentiated Day 7 and Day 21 cells was characterized by ICC. Adherent cells and cellular aggregates were fixed in 4% paraformaldehyde (PFA) for 30 minutes at room temperature (RT). Fixed cells and aggregates were washed with phosphate buffered saline (PBS), and fixed aggregates were then sequentially placed in increasing concentrations of sucrose solution (10%, 20%, and 30% weight/volume) for 30 minutes at RT, 30 minutes at RT, and overnight at 4° C., respectively. Following sucrose replacement, aggregates were embedded in Tissue-Tek Optimal Cutting Temperature (OCT) solution (Sakura Finetek USA #4583) and frozen at −80° C. OCT-embedded aggregates were warmed to −20° C., cut into 30 μm sections using a cryostat (model CM3050 S, Leica Biosystems, Buffalo Grove, IL, USA), and mounted onto poly-L-lysine (Sigma-Aldrich #P4707) coated glass slides.

To perform immunocytochemical staining, fixed adherent cells and slide-mounted aggregate sections were permeabilized and blocked in blocking solution consisting of 0.1% Triton™ X-100/2% normal goat serum/1% bovine serum albumin in PBS for 2 hours at room temperature (RT). Following permeabilization and blocking, adherent cells and aggregate sections were incubated overnight at 4° C. in blocking solution without Triton™ X-100 and containing primary antibodies specific to protein markers of interest, including PAX6 (BD Pharmingen #561462 or BioLegend #901301) to detect neuroectoderm progenitors, and AP2 (Developmental Studies Hybridoma Bank-DSHB #3B5), PAX3 (DSHB #Pax3), and PAX7 (DSHB #Pax7) to detect dorsal spinal cord progenitor cells. Adherent cells and aggregate sections were then washed three times with PBS followed by incubation with secondary antibodies specific to the chosen primary antibodies and 4′,6-diamidino-2-phenylindole (DAPI) counter-stain in blocking solution without Triton™ X-100 for 1 hour at RT protected from light. Adherent cells and aggregate sections were washed three times with PBS and imaged using an IN Cell Analyzer 2000 (GE Healthcare, Pittsburgh, PA, USA).

After 7 days of differentiation, the adherent cell population from two representative experiments expressed PAX6, a protein marker characteristic of neuroectoderm progenitor cells and also expressed the dorsal spinal cord progenitor markers, AP2, PAX3, and PAX7.

Aggregates were sectioned and stained for dorsal progenitor markers AP2, PAX3, and PAX7, as well as pan-neural progenitor marker PAX6. While these early progenitor cells were still present at Day 21, also present was a distinct glial population expressing the oligodendrocyte progenitor marker NG2.

For human embryonic stem cells differentiated through Day 42 into oligodendrocyte progenitor cells, protein marker expression in the resulting single cell population was characterized by both flow cytometry and ICC. To characterize protein marker expression of the oligodendrocyte progenitor cells by ICC, staining was carried out as described above for slide-mounted aggregate sections, except permeabilization was performed with 100% methanol for 2 minutes at RT, and blocking solution consisted of 10% fetal bovine serum in PBS.

Based on ICC data for the Day 42 oligodendrocyte progenitor cells, the resulting single cell population from two representative experiments expressed the oligodendrocyte progenitor cell marker NG2 and reduced expression of the dorsal spinal cord progenitor cell marker AP2.

To quantify cell surface markers on Day 42 by flow cytometry, cells were thawed in Thaw Medium (10% fetal bovine serum in DMEM medium), centrifuged and resuspended in Stain Buffer (2% fetal bovine serum/0.05% sodium azide in PBS). Cells were incubated with primary antibodies specific to markers of interest, including NG2 (Invitrogen #37-2300), PDGFRα (BD Biosciences #563575), GD3 (Millipore #MAB2053), A2B5 (BD #563775), CD49f (Millipore #CBL458P), EpCAM (Dako #M080401-2) and CLDN6 (Thermo Fisher #MA5-24076), and their isotype controls for 30 minutes on ice. Cells were washed with Stain Buffer to remove unbound antibodies; in the case of unconjugated antibodies, cells were then incubated with appropriate fluorophore-conjugated secondary antibodies for 30 minutes on ice. Cells were washed and propidium iodide was then added to demark dead cells. In some cases, cells were cultured overnight at 37° C./5% CO2 in tissue culture vessels coated with Matrigel (Corning #356231) to recover protein markers that exhibited sensitivity to the Day 42 harvesting procedure described in Example 5, and were then harvested with TrypLEIM Select (Thermo Fisher #A12859-01) and stained for flow cytometry analysis as described above. All cells were analyzed on an Attune NxT (Thermo Fisher, Waltham, MA, USA) flow cytometer. To calculate the percentage of cells expressing a given protein marker, dead cells staining with propidium iodine were gated and the number of viable cells bound to the corresponding antibody was expressed as a fraction of the total number of cells analyzed after correcting for the number of cells that exhibited non-specific binding to the isotype control antibody.

Table 5 shows representative flow cytometry data for Day 42 oligodendrocyte progenitor cells generated in accordance with the methodology described in Example 5. As shown for two representative runs, a high proportion of cells in the resulting cell population expressed characteristic oligodendrocyte markers, including NG2 and PDGFRα. In addition, non-OPC markers were minimally detected in the resulting population, including the neural progenitor/epithelial marker CD49f and the epithelial markers CLDN6 and EpCAM.

TABLE 5
Representative flow cytometry data for oligodendrocyte
progenitor cells produced by a method in
accordance with the present disclosure.

| | OPC/Pre-OPC markers | | | | Non-OPC markers | | |
|---|---|---|---|---|---|---|---|
| | NG2 | PDGFRα | GD3 | A2B5 | CD49f | CLDN6 | EpCAM |
| Run 1 | 92% | 85% | 66% | 39% | 3% | 0% | 0% |
| Run 2 | 98% | 95% | 63% | 89% | 0% | 0% | 2% |

The cell population generated by the methodology described in the present disclosure resulted in higher proportion of cells positive for oligodendrocyte progenitor cell marker NG2 and reduced expression of non-OPC markers CD49f, CLDN6, and EpCAM when compared to OPCs that are currently in clinical testing to treat spinal cord injury and that were generated using another method (Priest C A, Manley N C, Denham J, Wirth E D 3rd, Lebkowski J S. Preclinical safety of human embryonic stem cell-derived oligodendrocyte progenitors supporting clinical trials in spinal cord injury. Regen Med. 2015 November; 10 (8): 939-58; Manley N C, Priest C A, Denham J, Wirth E D 3rd, Lebkowski J S. Human Embryonic Stem Cell-Derived Oligodendrocyte Progenitor Cells: Preclinical Efficacy and Safety in Cervical Spinal Cord Injury. Stem Cells Transl Med. 2017 October; 6 (10): 1917-1929).

### Example 7—Characterization of Differentiated Cell Populations by Gene Expression Profiling

Gene expression profiling can be used to characterize the cellular phenotype of the starting pluripotent cell population and each stage of differentiation, including the generation of neuroectoderm progenitor cells, glial progenitor cells, and oligodendrocyte progenitor cells. Gene expression profiling includes both global transcriptome profiling, using such methods as microarray and RNA-seq, and targeted gene profiling using methods of increased sensitivity such as quantitative real-time PCR (qPCR). To perform gene expression profiling, cells were lysed in Qiagen RLT Lysis Buffer (Qiagen #79216), and RNA was purified using Qiagen RNeasy Mini Kit (Qiagen #74106) according to the manufacturer's guidelines. For qPCR-based analysis, purified RNA was then converted to cDNA according to standard methods using the Invitrogen Superscript IV VILO Mastermix (Thermo Fisher Scientific #11756050) according to the manufacturer's guidelines. The relative expression level of target genes and reference housekeeping genes was then quantified using gene-specific primer-probe sets (Applied Biosystems Taqman Gene Expression Assays, Thermo Fisher Scientific #4331182) according to the manufacturer's guidelines. To determine relative expression levels of a given set of target genes, PCR reactions were performed on the ABI 7900HT Real-Time Sequence Detection System (Applied Biosystems), the BioMark HD System (Fluidigm) or equivalent. Each target gene was normalized to one or multiple reference genes, such as GAPDH, to determine its relative expression level.

Table 6 shows qPCR results from two representative experiments measuring expression of pluripotency genes, neuroectoderm progenitor cell genes, glial progenitor cell genes, dorsal spinal cord progenitor cell genes, ventral spinal cord progenitor cell genes, and oligodendrocyte progenitor cell genes in cell populations generated by methods in accordance with the present disclosure. RNA samples were collected at the following time points: prior to differentiation (Day 0), following differentiation to neuroectoderm progenitors (Day 7), following differentiation to glial progenitors (Day 21), and following differentiation to oligodendrocyte progenitors (Day 42). RNA samples were processed for qPCR using the methods described above. A selected panel of genes indicative of each differentiation state were quantified, including: three pluripotency genes (NANOG, LIN28A, SOX2), three neuroectoderm progenitor genes (PAX6, HES5, ZBTB16), three glial progenitor genes (CACGN4, DCC, FABP7), and three oligodendrocyte progenitor genes (CSPG4, PDGFRα, DCN). For each gene, normalized ACT values were calculated using the average of five housekeeping genes (ACTB, GAPDH, EP300, PGK1, SMAD1), and fold expression relative to baseline (expression below the limit of quantification) was calculated using the ΔΔCT method.

TABLE 6
qPCR analysis of gene markers for pluripotency, neuroectoderm
progenitor cells (NPCs), dorsal spinal cord progenitor cells,
ventral spinal cord progenitor cells, glial progenitor cells (GPCs),
and oligodendrocyte progenitor cells (OPCs) in H1 uhESCs
differentiated into OPCs in accordance with the present disclosure.

| | | | | | | | |
|---|---|---|---|---|---|---|---|
| Run 1 | Run 2 | Run 1 | Run 2 | Run 1 | Run 2 | Run 1 | Run 2 |
| Day | Day | Day | Day | Day | Day | Day | Day |
| 0 | 0 | 7 | 7 | 21 | 21 | 42 | 42 |
| uhESC | uhESC | NEPC | NEPC | GPC | GPC | OPC | OPC |

Pluripotency genes

| | | | | | | | | |
|---|---|---|---|---|---|---|---|---|
| NANOG | 158 | 119 | 4 | 3 | 2 | 1 | 2 | 1 |
| LIN28A | 650 | 1684 | 233 | 626 | 5 | 1 | 2 | 1 |
| SOX2 | 480 | 325 | 532 | 243 | 767 | 262 | 2 | 3 |

Neuroectoderm progenitor cell (NPC) genes

| | | | | | | | | |
|---|---|---|---|---|---|---|---|---|
| PAX6 | 1 | 1 | 305 | 250 | 218 | 32 | 2 | 2 |
| HES5 | 1 | 1 | 65 | 71 | 275 | 298 | 6 | 1 |
| ZBTB16 | 1 | 1 | 110 | 120 | 50 | 31 | 2 | 1 |

Dorsal spinal cord progenitor cell genes

| | | | | | | | | |
|---|---|---|---|---|---|---|---|---|
| TFAP2A | 1 | 1 | 67 | 84 | 169 | 215 | 69 | 20 |
| PAX3 | 1 | 1 | 100 | 300 | 91 | 118 | 2 | 7 |
| PAX7 | 1 | 1 | 74 | 178 | 104 | 89 | 19 | 6 |

Ventral spinal cord progenitor cell genes

| | | | | | | | | |
|---|---|---|---|---|---|---|---|---|
| OLIG2 | 1 | 1 | 1 | 1 | 2 | 1 | 2 | 1 |
| NKX2-2 | 1 | 1 | 1 | 1 | 2 | 1 | 2 | 1 |

Glial progenitor cell (GPC) genes

| | | | | | | | | |
|---|---|---|---|---|---|---|---|---|
| CACNG4 | 18 | 11 | 61 | 48 | 268 | 317 | 32 | 15 |
| DCC | 1 | 2 | 17 | 51 | 34 | 324 | 2 | 5 |
| FABP7 | 6 | 10 | 7 | 9 | 17 | 51 | 2 | 4 |

Oligodendrocyte progenitor cell (OPC) genes

| | | | | | | | | |
|---|---|---|---|---|---|---|---|---|
| CSPG4 | 5 | 3 | 5 | 10 | 6 | 49 | 126 | 111 |
| PDGFRα | 1 | 2 | 19 | 20 | 23 | 185 | 584 | 481 |
| DCN | 1 | 1 | 1 | 2 | 285 | 302 | 1080 | 1136 |

Referring to Table 6, differentiation of uhESCs for seven days by a method in accordance with the present disclosure resulted in a gene expression profile that was consistent with neuroectoderm progenitor cells, including downregulation of NANOG, and expression of LIN28A, SOX2, PAX6, HES5, and ZBTB16.

In addition, the neuroectoderm progenitor cells generated after seven days of differentiation exhibited a phenotype that was consistent with dorsal spinal cord progenitor cells based on expression of the dorsal markers TFAP2A (also known as AP2), PAX3, and PAX7. As further evidence of a dorsal spinal cord progenitor cell phenotype, the resulting neuroectoderm progenitor cells did not express the ventral spinal cord progenitor cell markers OLIG2 or NKX2-2, whose expression require activation of the sonic hedgehog signaling pathway.

After 21 days of differentiation, the resulting cell population exhibited a gene expression profile that was consistent with glial progenitor cells, including downregulation of pluripotency and neuroectoderm progenitor cell markers and induction of CACNG4, DCC (also known as the netrin receptor), and FABP7. As further evidence of a glial progenitor phenotype, the resulting Day 21 cells exhibited sustained expression of HES5, which in addition to its expression in neuroectoderm progenitor cells/neural progenitor cells, HES5 has also been shown to promote the neural to glial progenitor switch in the mammalian developing central nervous system. In addition, the resulting Day 21 glial progenitor cells exhibited sustained expression of the dorsal spinal cord progenitor markers, TFAP2A, PAX3 and PAX7, providing further evidence of derivation from dorsally-patterned neural progenitors.

Following 42 days of differentiation in accordance with the methods described in the present disclosure, the resulting cell population expressed markers consistent with oligodendrocyte progenitors, including downregulation of both the earlier lineage markers and dorsal spinal cord progenitor markers, and induction of CSPG4 (also known as NG2), PDGFRα, and DCN.

### Example 8—Differentiation of Human Embryonic Stem Cells to Dorsal Neuroectoderm Progenitor Cells Using Alternative Small Molecule Inhibitors of MAPK/ERK and BMP Signaling

In addition to the small molecule inhibitors used in Example 3 (PD0325901 and Dorsomorphin), alternative small molecule inhibitors of MAPK/ERK and BMP signaling were tested for their ability to differentiate human embryonic stem cells into dorsal neuroectoderm progenitors. Table 7 lists the alternative small molecule inhibitors that were tested. Each condition was tested in duplicate wells of a 6-well tissue culture plate.

TABLE 7
Small molecule inhibitors used to differentiate human embryonic stem
cells into dorsal neuroectoderm progenitors.

| | Concentration | Primary | Vendor |
|---|---|---|---|
| Inhibitor | tested | binding target(s) | catalogue # |
| MEK-ERK inhibitors | | | |
| PD0325901 | 10 μM | MEK1, MEK2 | ApexBio # A3013 |
| AZD6244 | 10 μM | MEK1, MEK2 | ApexBio # A8207 |
| GSK1120212 | 10 μM | MEK1, MEK2 | ApexBio # A3018 |
| PD184352 | 10 μM | MEK1, MEK2 | ApexBio # A1792 |
| Cobimetinib | 10 μM | MEK1, MEK2 | ApexBio # A3321 |
| BMP inhibitors | | | |
| Dorsomorphin | 2 μM | ALK2, ALK3, | Sigma-Aldrich # |
| | | ALK6 | P5499 |
| ML347 | 2 μM | ALK2, ALK1 | ApexBio # B3688 |

On differentiation Day 7, cells were collected and processed for RNA extraction and gene expression profiling by qPCR as described in Example 7. For each gene, a normalized ACT value was calculated relative to the average of five housekeeping genes (ACTB, GAPDH, EP300, PGK1, SMAD1), and fold expression relative to baseline (expression below the limit of quantification) was calculated using the ΔΔCT method. Table 6 shows the average of fold expression value for biological duplicates of each small molecule combination (relative to baseline). Referring to Table 7, differentiation of uhESCs for seven days with each of the tested small molecule combinations resulted in downregulation of the pluripotency marker NANOG and a similar degree of maintained expression or induction of genes associated with a neuroectoderm progenitor cell phenotype, including LIN28A, SOX2, PAX6, HES5, and ZBTB16. In addition, each of the tested small molecule combinations resulted in a dorsal spinal cord progenitor phenotype based on expression of the dorsal markers, TFAP2A, PAX3, and PAX7, and a lack of expression of the ventral markers, OLIG2 and NKX2-2.

To obtain a more comprehensive comparison of the resulting Day 7 cellular phenotypes after treatment with each small molecule combination, Fluidigm qPCR was conducted using a 96 gene panel that consisted of known markers for pluripotency, neuroectoderm progenitor cells, neural tube patterning, glial progenitor cells, oligodendrocyte progenitor cells, neural crest cells, neurons, astrocytes, pericytes, Schwann cells, and epithelial cells. Comparison of the day 7 cellular phenotype for each alternative small molecule combination to the cellular phenotype generated by treatment with PD0325901 plus Dorsomorphin by regression plot of the normalized ACT values indicated that a similar overall cellular phenotype could be achieved with each of the small molecule combinations tested. Taken together, the results shown in Table 8 support that various combinations of: (i) a MAPK/ERK inhibitor, together with (ii) a BMP signaling inhibitor, (iii) in the absence of a SHH signaling activator, can be used to differentiate uhESCs to dorsal neuroectoderm progenitor cells, and further to glial progenitor cells and to oligodendrocyte progenitor cells using the methods of the present disclosure.

TABLE 8
qPCR analysis of gene markers for pluripotency and neuroectoderm
progenitor cells (NPCs) in H1 uhESCs differentiated into
NPCs using different combinations of small molecule inhibitors.

| | | | | | |
|---|---|---|---|---|---|
| PD- | AZD- | GSK- | PD- | Cobi- | PD- |
| 0325901 + | 6244 + | 1120212 + | 184352 + | metinib + | 0325901 + |
| Dorso | Dorso | Dorso | Dorso | Dorso | ML347 |

Pluripotency genes

| | | | | | | |
|---|---|---|---|---|---|---|
| NANOG | 1 | 4 | 1 | 1 | 1 | 1 |
| LIN28A | 548 | 618 | 606 | 635 | 504 | 488 |
| SOX2 | 370 | 484 | 254 | 401 | 201 | 202 |

Neuroectoderm progenitor cell genes

| | | | | | | |
|---|---|---|---|---|---|---|
| PAX6 | 363 | 539 | 258 | 417 | 173 | 156 |
| HES5 | 97 | 100 | 68 | 91 | 36 | 36 |
| ZBTB16 | 135 | 149 | 145 | 171 | 134 | 120 |

Dorsal spinal cord progenitor cell genes

| | | | | | | |
|---|---|---|---|---|---|---|
| TFAP2A | 67 | 61 | 84 | 43 | 100 | 166 |
| PAX3 | 112 | 99 | 180 | 84 | 193 | 158 |
| PAX7 | 156 | 106 | 186 | 100 | 148 | 209 |

Ventral spinal cord progenitor cell genes

| | | | | | | |
|---|---|---|---|---|---|---|
| OLIG2 | 1 | 1 | 1 | 1 | 1 | 1 |
| NKX2-2 | 1 | 1 | 1 | 1 | 1 | 1 |

TABLE 9
Inclusion and Exclusion Criteria

```
Inclusion Criteria
Participants were eligible for the study if all of the following inclusion
criteria were met prior to dosing of LCTOPC1:
  1. Neurogically complete, traumatic SCI (American Spinal Injury Association
Impairment Scale A); spared motor or sensory function <5 levels below the
relevant sensory or motor (right or left) level.
  2. Single neurological level from T-3 through T-11.
  3. From 18 through 65 years of age at time of injury.
  4. Single spinal cord lesion on a post-stabilization MRI scan, with sufficient
visualization of the spinal cord for 30 mm above and below the injury
epicenter to enable post-injection safety monitoring.
  5. Informed consent for this protocol and the long-term follow-up protocol
provided and documented (i.e., signed informed consent forms) no later than
11 days following injury.
  6. Able to participate in elective surgical procedure to inject LCTOPC1 7 to
14 days following SCI.
Exclusion Criteria
Participants were not eligible for the study if any of the following exclusion
criteria were not met prior to dosing of LCTOPC1:
  1. Spinal cord injury due to penetrating trauma.
  2. Traumatic anatomical transection or laceration of the spinal cord based on
prior surgery or MRI.
  3. Spinal cord lesion with anteroposterior diameter of the spinal cord <2 mm
at point of maximal compression on a midline sagittal image from a post-
stabilization MRI.
  4. Any comcomitant injury that could interfere with the performance,
interpretation or validity of neurological examinations, such as multiple spinal
cord lesions, lumbar plexus injury, cauda equina injury, or traumatic brain
injury.
  5. Any treatment or pre-existing condition that could interfere with the
performance, interpretation or validity of neurological examinations,
such as polyneuropathy, focal or multi-focal neuropathy, myelopathy or
radiculopathy.
  6. Inability to communicate effectively with neurological examiner such
that the validity of patient data could have been compromised.
  7. Significant organ damage or systemic disease that would have created
an unacceptable risk for surgery or immunosuppression.
  8. Concomitant use at baseline of other immunosuppressive agents, such as
corticosteroids, that would have created an unacceptable risk for additional
immunosuppression with tacrolimus.
  9. Need for mechanical support of ventilation (ventilator, continuous positive
airway pressure, bi-level positive airway pressure), excluding supplemental
oxygen, at baseline.
10. History of any malignancy.
11. Pregnant or nursing women. Female participants of childbearing potential
agreed to prevent pregnancy by the use of contraception for 365 days following
LCTOPC1 injection; male participants agreed to use contraception to prevent
pregnancy in any female partners of child bearing potential for 365 days
following LCTOPC1 injection.
12. Positive blood test for antibodies to human immunodeficiency virus
types 1 or 2, antibodies to hepatitis B virus core antigen, or antibodies to
hepatitis C virus.
13. Panel reactive antibodies (PRA) ≥20%; if a site lab reported PRA only
for human leukocyte antigen (HLA) Class I or separately for HLA Class I and
II, exclusion was based solely on the PRA fo HLA Class I.
14. Serum creatinine above the established limit for the normal range at
individual study center laboratories at baseline.
15. Liver function tests >2× the established upper limit for the normal range
at individual study center laboratories at baseline.
16. Hematocrit ≤27% at baseline.
17. Positive blood cultures (48-hour culture results at Day −1).
18. Active untreated viral, fungal or bacterial infeciton at baseline.
19. Evidence of surgical site infection at intended LCTOPC1 injection site
at baseline.
20. Temperature ≥38.6° C. at 2 time points from Day −1 through Day 0
prior to surgery for LCTOPC1 injection.
21. Body mass index >35 kg/m2 or weight >300 pounds.
22. Active participation in another experimental procedure/intervention.
23. Psychoactive substance use disorder (as defined by the Diagnostic and
Statistical Manual of Mental Disorders, Fourth Edition) at any time during
the 3 months preceding study entry.
24. History of major depression, schizophrenia, paranoia, or other psychotic
disorder as defined by Diagnostic and Statistical Manual of Mental Disorders,
Fourth Edition.
25. Participant who, in the opinion of the investigator, was unlikely to return
for all follow-up visits as specified in the protocol.
26. Any condition that, in the judgement of the investigator, would have
precluded successful participation in the study.
```

TABLE 10
Schedule of Events from Screening to Day 90

| | Visit: | | | | | | | | | | | | | |
|---|---|---|---|---|---|---|---|---|---|---|---|---|---|---|
| | Screen | Day −3 | Baseline | Day −11 | Day 0 | Follow-up 2 | | | | | | | | |
| | Procedures Day (window): | | | | | | | | | | | | | |
| | | | | | | | | 7 | 7 | 30 | 30 | 60 | 60 | 90 |
| | −11 to −3 | −3 | −2 | −1 | 0 | 1 | 1 to 7 | (±1) | to 30 | (±3) | to 60 | (±7) | to 90 | (±7) |
| Informed consent3 | X | | | | | | | | | | | | | |
| Demographics and | X | | | | | | | | | | | | | |
| medical history | | | | | | | | | | | | | | |
| Physical exam | X4 | | X1.5 | | | X5 | | X5 | | X5 | | X5 | | X5 |
| Vital signs | X | | X1 | X | X | X | | X | | X | | X | | X |
| Neurological exam | | | X | | | | | X | | X | | X | | X |
| ISNCSCI exam | X | | X1 | X6 | | | | X | | X | X7 | X | X7 | X |
| UAB-IMR | | | X | | | | | X | | X | | X | | X |
| Pain questionnaire | | | | | | | | | | X | | | | X |
| Bowel and bladder | | | | | | | | | | X8 | | | | |
| questionnaire and | | | | | | | | | | | | | | |
| SCIM | | | | | | | | | | | | | | |
| Electrocardiogram | X | | | | | | | X | | | | | | |
| MRI | X9 | | | | | | | X10 | | X11 | | X10 | | X11 |
| Pregnancy test, if | X | | | | | | | | | | | | | |
| applicable | | | | | | | | | | | | | | |
| HIV, Hep B, | X | | | | | | | | | | | | | |
| Hep C, and panel | | | | | | | | | | | | | | |
| reactive antibodies | | | | | | | | | | | | | | |
| Hematology | X | | X1 | | | X | | X | | X | | X | | X |
| Blood chemistry | X | | X1 | | | X | | X | | X | | X | | X |
| panel 112 | | | | | | | | | | | | | | |
| Blood chemistry | | | | | | | X13.15 | | X13.15 | | X13.15 | X16 | | |
| panel 213 | | | | | | | | | | | | | | |
| Blood chemistry | | | | | | | | | X14.15 | | X14.15 | X16 | | |
| panel 314 | | | | | | | | | | | | | | |
| 48-hour blood | | X | | | | | | | | | | | | |
| cultures | | | | | | | | | | | | | | |
| HLA typing | | | X | | | | | | | | | | | |
| Fasting blood | | | X | | | | | | | X | | X | | |
| glucose | | | | | | | | | | | | | | |
| Tacrolimus level | | | | | | | X17 | | X17 | | X18 | X19 | | |
| Blood for immune | | | X | | | X | | X | | X | X20 | X | X20 | X |
| response (shipped) | | | | | | | | | | | | | | |
| Blood for | | | X | | | | | | | X | | | | |
| xenotransplantation | | | | | | | | | | | | | | |
| (shipped) | | | | | | | | | | | | | | |
| Withhold | | | | X21 | | | | | | | | | | |
| pharmocological | | | | | | | | | | | | | | |
| DVT prophylaxis | | | | | | | | | | | | | | |
| Restart | | | | | | X22 | | | | | | | | |
| pharmacological | | | | | | | | | | | | | | |
| DVT prophylaxis | | | | | | | | | | | | | | |
| Begin tacrolimus | | | | | X23 | | | | | | | | | |
| Taper tacrolimus | | | | | | | | | | | X24 | | | |
| dose | | | | | | | | | | | | | | |
| Discontinue | | | | | | | | | | | | X | | |
| tacrolimus | | | | | | | | | | | | | | |
| CSF via LP | | | | | X25.26 | | | | | | | X26 | | |
| (10 mL) | | | | | | | | | | | | | | |
| AST-OPC1 | | | | | X | | | | | | | | | |
| Injection | | | | | | | | | | | | | | |
| Concomitant | X | | X1 | X | X | X | | X | | X | | X | | X |
| medications | | | | | | | | | | | | | | |
| Adverse events | X | | X1 | X | X | X | | X | | X | | X | | X |

ALT-alanine aminotransferase;
AST-aspartate aminotransferase,
CSF-cerebrospinal fluid;
DVT-deep vein thrombosis;
Gd-DTPA-gadolinium dietheylenetriamine pentaacetic acid;
HIV-human immunodeficiency virus;
Hep-hepatitis;
HLA-human leukocyte antigen;
ISNCSCI-Internal Standards for Neurological Classification.
1If the day of injection was delayed, the following baseline tests and procedures were to be repeated on the new Day 1: brief physical examination, vital signs, ISNCSCI examination, hematology, blood chemistry panel 1, concomitant medications, and adverse events.
2If a participant’s participation terminated between Day 0 and Day 60, all tests and procedures scheduled for the Day 60 visit were collected at the final visit prior termination from the study, if possible. If a participant’s participation terminated between Day 60 and Day 365, all tests and procedures scheduled for the Day 365
3Informed consent was collected for treatment and long-term follow-up.
4Complete examination.
5Brief examination.
6Rectal examination only to confirm completeness of injury.
7A single pre-tapering ISNCSCI examination was performed between Day 42 and Day 46. Additional ISNCSCI examinations were performed twice weekly from Days 46 to 60, and weekly from Days 60 to 90. For those participants who exhibited neurological improvement through Day 46 greater than what would have been expected
8SCIM was collected as a questionnaire (versus observation of activities).
9A screening/baseline MRI was obtained between Day −5 and Day −3, but no earlier than 4 days after SCI. This MRI included the brain, cerebellum and entire spinal cord, with and without Gd-DTPA contrast. If surgery for AST-OPC1 injection was subsequently delayed for more than 3 days, then a repeat MRI of the T-spine, without
10MRI included the spinal core and cerebellum, with and without Gd-DTPA contrast.
11MRI included the brain in addition to the routine imaging of the spinal cord and cerebellum, with and without Gd-DTPA contrast.
12Blood chemistry panel 1: serum albumin, alkaline phosphatase, blood urea nitrogen, total bilirubin, chloride, serum creatinine, glucose, potassium, total serum protein, sodium, AST, ALT.
13Blood chemistry panel 2: serum creatinine, postassium, magnesium, phosphate, and iodized clacium were obtained daily for 1 week after initiation of tacrolimus, twice per week from Day 7 to Day 30, and once per week from Day 31 to Day 60.
14Blood chemistry panel 3: serum AST, ALT, and total bilirubin were measured 1 week after initiation of tacrolimus and then once per week until Day 60.
15When overlap with routine study visits occurred (Day 1, 7, 30, or 60), the same blood sample may have been used for both purposes. For example-panel 1 consisted of serum albumin, alkaline phosphatase, blood urea nitrogen, total bilirubin, chloride, serum creatinine, glucose, potassium, total serum protein, sodium, AST.
16Serum creatinine, potassium, magnesium, phosphate, ionized calcium, AST, ALT, and total bilirubin were obtained on Day 60, regardless of the time elapsed since the immediately preceding chemistry panel was obtained.
17Whole blood levels (if given intravenously) or trough levels (if given orally) of tacrolimus were measured within 3 days of initiation of treatment and twice per week thereafter until Day 30. If tacrolimus administration was changed from intravenous to oral, or if the dosage was adjusted, a trough blood level was obtained.
18Whole blood trough levels of tacrolimus were measured once per week from Day 30 to Day 60. If the dosage was adjusted during this time (other than scheduled tapering from Days 46-60), a trough blood level was obtained within 3 days, after which blood level monitoring once per week resumed through Day 60.
19Tacrolimus blood level and all serum samples were obtained on Day 60, regardless of the time elapsed since the previous blood collection.
20Additional blood samples for immune response monitoring were obtained weekly from Days 45 to 60 and 60 to 90. When overlap with the Day 60 or 90 visit occured, the same blood sample could have been used for both purposes.
21Pharmacological DVT prophylaxis may have been stopped on Day −1 in preparation for surgery, depending on the prophylaxis used and standards of care at the clinical site.
22Pharmacological DVT prophylaxis was re-started following the procedure to implant AST-OPC1, depending on the prophylaxis used and standards of care at the clinical site.
23Began 6 to 12 hours post-injection of AST-OPC1.
24At Day 46, the tacrolimus dose was decreased by 50% (rounded to the nearest 0.5 mg, since this was the smalled capsule size available). At Day 53, the tacrolimus dose was decreased by another 50% (rounded to the nearest 0.5 mg). If the rounded total daily dose was 0.5 mg or lower, the participant received 0.5 mg once per
25CSF collection on Day 0 occured via LP after anesthesia but prior to surgicall incision.
26The volume required at individual study sites for the following tests were sent to the hospital laboratory: white blood cell count, glucose, total protein, oligoclonal banding, myelin basic protein, and immunoglobulin G index. The remainder of the sample was processed and sent to an outside laboratory.

TABLE 11
Schedule of Events from Day 120 to Day 365.

| | Procedures | | | |
|---|---|---|---|---|
| | | Follow-up | | Final Visit1 |
| | Day (window): | | | |
| | 120 (±7) | 180 (±14) | 270 (±14) | 365 (±14) |
| Informed consent for companion long-term, | | | | X |
| follow-up study | | | | |
| Physical examination | X2 | X2 | X2 | X3 |
| Vital signs | X | X | X | X |
| Neurological examination | X | X | X | X |
| ISNCSCI examination | X | X | X | X |
| UAB-IMR | X | X | X | X |
| Pain questionnaire | | X | | X |
| Bowel and bladded questionnaire | | X | | X |
| SCIM | | X4 | | X4 |
| MRI | X5 | X6 | X5 | X6 |
| Hematology | X | X | X | X |
| Blood chemistry-panel 17 | X | X | X | X |
| Blood samples for immune response (shipped) | X | X | X | X |
| Blood samples for xenotransplantation (shipped) | | | | X |
| Concomitant medications | X | X | X | X |
| Adverse events | X | X | X | X |

ALT = alanine aminotransferase;
AST = aspartate aminotransferase;
Gd—DTPA = gadolinium diethylenetriamine pentaacetic
1If a participant's participation terminated between Day 60 and Day 365, all tests and procedures scheduled for the Day
2Brief examination.
3Complete examination.
4SCIM was collected as a questionnaire (versus observation of activities).
5MRI included the spinal cord and cerebellum, with and without Gd—DTPA contrast.
6MRI included the brain in addition to the routine imaging of the spinal cord and cerebellum, with and without Gd—DTP.
7Serum albumin, alkaline phosphatase, blood urea nitrogen, total bilirubin, chloride, serum creatinine, glucose, potassium

### Example 9—a Phase 1/2a Dose Escalation Study of AST-OPC1 in Subjects with Subacute Cervical Spinal Cord Injury

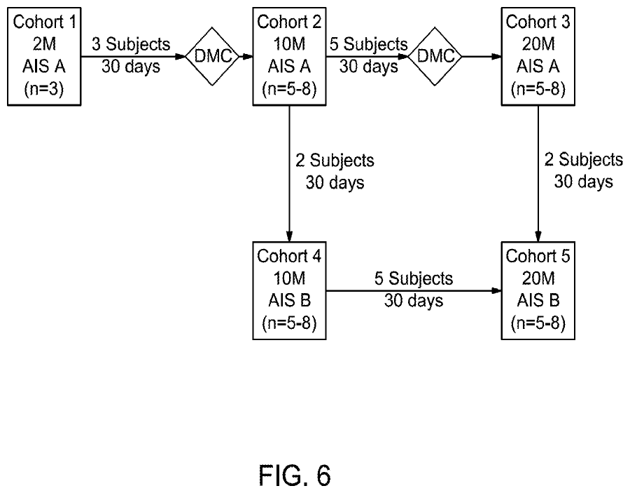

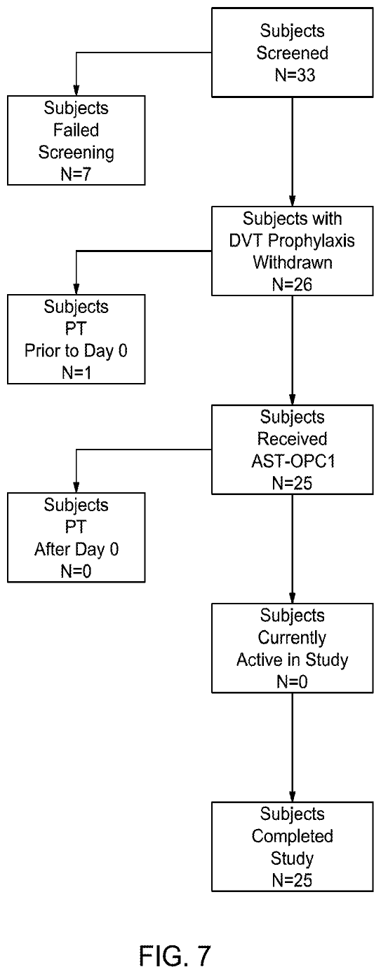

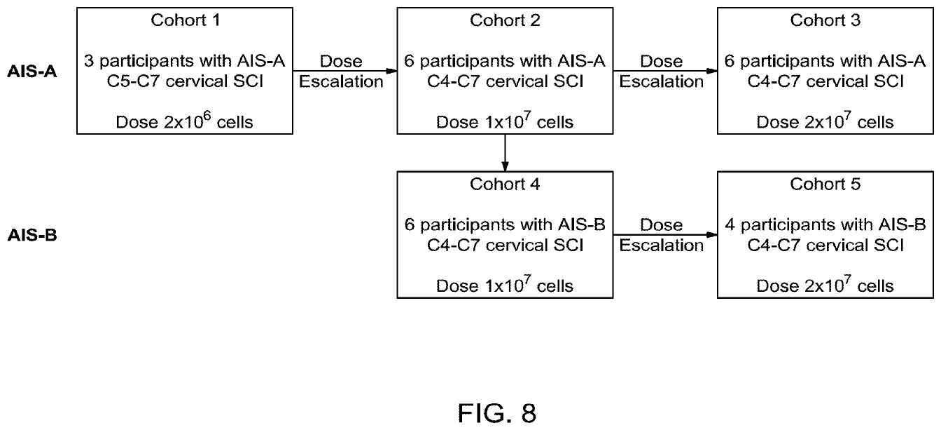

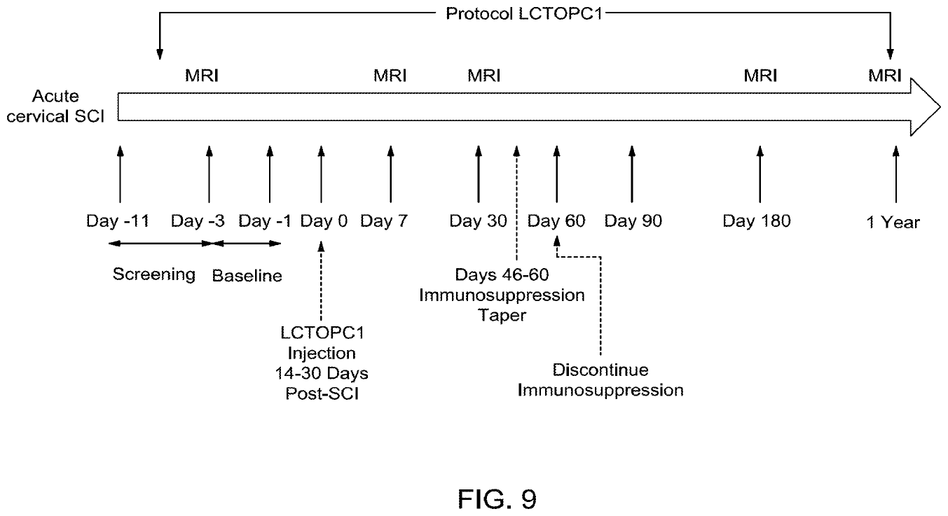

Study Design. The trial design was an open-label, staggered dose escalation, cross-sequential, multicenter study. Three sequential, escalating doses of AST-OPC1 were administered at a single time point between 21 and 42 days post-injury to subjects with subacute cervical spinal cord injuries (SCI). Subjects received AST-OPC1 via direct intra-spinal injection using a Syringe Positioning Device (SPD). To prevent rejection of engraftment, low dose tacrolimus was initiated 6 to 12 hours after and intra-spinal injection, and continued for 46 days, tapered from Day 46 to Day 60, and was discontinued at Day 60. Subjects were followed for 1 year following administration of AST-OPC1 under this protocol. Male or female subjects from 18 to 69 years of age at time of consent with sensorimotor complete, traumatic SCI (American Spinal Injury Association Impairment Scale A) or sensorimotor incomplete, traumatic SCI (American Spinal Injury Association Impairment Scale B). Subjects had a single neurological level of injury (NLI) from C-5 through C-7 or a C4 NLI with an upper extremity motor score (HEMS)≥1. There was a single spinal cord lesion on a post-stabilization magnetic resonance imaging (MRI) scan, with sufficient visualization of the spinal cord injury epicenter and lesion margins to enable post-injection safety monitoring. Subjects were able to participate in an elective surgical procedure to inject AST-OPC1 21 to 42 days following SCI. Subjects received a single dose of either 2×10⁶, 1×10⁷, or 2×10⁷ AST-OPC1 viable cells by injection, administered 21 to 42 days following SCI. The product was delivered intraoperatively into the spinal cord using the Syringe Positioning Device. The AST-OPC1 batch numbers used in this study were M08D1A, M22D1A and M25D1A. Single administration of AST-OPC1 was provided with 1 year of follow-up. Schematics of the planned study timeline, and subject screening and treatment, are provided in FIG. 6 and FIG. 7, respectively. A schematic diagram of the final open-label, staggered dose-escalation, multicenter Phase 1/2a clinical trial is shown in FIG. 8. An overview of study visits for the one-year protocol is presented in the study schema in FIG. 9.

A neurological examination was completed using the standardized International Standards for Neurological Classification of Spinal Cord Injury (ISNCSCI) examination for motor and sensory testing and for designation of the American Spinal Injury Association impairment scale. The ISNCSCI is used for efficacy with respect to improved motor function in the extremities, improved sensory function, and/or a descending neurological level. Safety assessments included physical examination, vital signs, ISNCSCI neurological examination, pain questionnaire (International Spinal Cord Injury Pain Basic Data Set, ISCIPBDS), electrocardiogram (ECG), Magnetic Resonance Imaging (MRI), laboratory tests, concomitant medications, and adverse events (AEs). Results for the ISNCSCI were listed for each subject by scheduled visit and analyzed by change in motor level and motor scores as well as other exploratory assessments of arm/hand function, self-care ability and overall volitional performance. Adverse events were tabulated by system organ class and by preferred term within system organ class according to the Medical Dictionary for Regulatory Activities (MedDRAR) Version 18.0. Statistical analysis of the safety data was performed using descriptive statistical methods including AEs incidence, severity and relatedness to AST-OPC1, to the injection procedure for product administration, and concomitant immunosuppression with tacrolimus. The number of laboratory assessments (hematology, clinical chemistry) that were below, within, or above the normal laboratory reference range were summarized for each analyte at scheduled study visits. Vital signs were summarized by calculating the mean, standard deviation, median, and range of values at each of the protocol-specified time points.

Results. The study enrolled 26 subjects. Twenty-five subjects were administered AST-OPC1 at 5 study sites. All 25 subjects completed 1 year of follow-up. The 25 subjects who were administered AST-OPC1 ranged in age from 18 to 62 years, 21 subjects were male and 4 were female, and the majority were Caucasian (22 subjects). Vehicular accident was the cause of SCI in 8 subjects. None of 25 treated subjects exhibited evidence of unexpected neurological deterioration on ISNCSCI examinations after completing 1-year follow-up. The safety data indicates that AST-OPC1 can be safely administered to subjects in the subacute period after cervical SCI. The injection procedure and the low-dose temporary immunosuppression regimen were well tolerated. The 25 subjects who received AST-OPC1 completed 1 year of follow-up and showed no evidence of neurological deterioration or adverse findings on MRI scans.

Table 12 below shows the AST-OPC1 dose cohorts and injection preparations used in the study. AST-OPC1 is a cryopreserved cell population containing a mixture of oligodendrocyte progenitor cells and other characterized cell types that are obtained following differentiation of undifferentiated human embryonic stem cells. At the time of cryopreservation, each vial contained 7.5×10⁶ viable cells in 1.2 mL of cryopreservation medium. The components of the cryopreservation medium were the following: Glial Progenitor Medium (GPM)-86% (v/v) [98% DMEM/F12 with GlutaMAX supplement, 1.9% B-27 supplement and 0.1% T3]; 25% Human serum albumin (HSA)-3.6% (v/v); 1 M HEPES-0.9% (v/v); DMSO-9.5% (v/v).

TABLE 12
Dose Cohorts and Injection Preparation.

| | | | Volume | | Total |
|---|---|---|---|---|---|
| | Dose | Con- | per | Number | Volume |
| | (No. of | centration | Injection | of | Administered |
| Cohort | cells) | (cells/μL) | (μL) | Injections | (μL) |
| 1 | 2 × 10⁶ | 4 × 10⁴ | 50 | 1 | 50 |
| 2 | 1 × 10⁷ | 2 × 10⁵ | 50 | 1 | 50 |
| 3 | 2 × 10⁷ | 2 × 10⁵ | 50 | 2 | 100 |
| 4 | 1 × 10⁷ | 2 × 10⁵ | 50 | 1 | 50 |
| 5 | 2 × 10⁷ | 2 × 10⁵ | 50 | 2 | 100 |

Dose selection and Timing. The first proposed dose of 2×10⁶ cells was evaluated for safety in a previous thoracic SCI trial. 2×10⁶ cells were used again in Cohort 1 in this trial to establish lack of complications due to the injection procedure. The increase from the first dose (2×10⁶ cells in 50 μL) to the second dose (1×10⁷ cells in 50 μL) only entails increasing the concentration of AST-OPC1 such that both of these doses were delivered via a single injection with a 50 μL volume. Therefore, the neurosurgeons consulted for this study viewed this initial dose escalation as a very small step with respect to the potential risks associated with the injection procedure. In addition, the safety of administering the second dose (1×10⁷ cells in 50 μL) was demonstrated in the uninjured pig cervical spinal cord at C6. This study confirmed the minimal expected tissue damage associated with injections into the uninjured spinal cord, and no evidence of efflux or cellular dissemination via the CSF was observed. The third dose represents an additional injection of 1×10⁷ cells in 50 μL at a second site within the lesion in a manner similar to that used for the rodent safety studies. This dose is within the 6 to 12× safety margin relative to the highest dose tested in the rat safety studies, particularly with respect to the total volume injected.

LCTOPC1 was supplied to the clinical sites in sterile, single-dose, single-use, 2.0 mL Corning™ cryovials. At the time of cryopreservation, each vial typically contained 7.5×10⁶ viable cells in 1.2 mL of cryopreservation medium. The components of the cryopreservation medium were the following: 1) Glial Progenitor Medium (GPM)—86% (v/v) [98% DMEM/F12 with GlutaMAX supplement, 1.9% B—27 supplement and 0.1% T3]; 2) 25% Human serum albumin (HSA)—3.6% (v/v); 3) 1 M HEPES—0.9% (v/v); 4) DMSO—9.5% (v/v). The cryopreserved drug product was thawed, washed, resuspended in the injection medium, and loaded into the injection syringe at the clinical sites.

LCTOPC1 is a cell population containing a mixture of oligodendrocyte progenitor cells (OPCs) and other characterized cell types that are obtained following differentiation of undifferentiated human embryonic stem cells (uhESCs). LCTOPC1 Drug Product (DP) is manufactured by a continuous process. Harvested LCTOPC1 Drug Substance is a transient intermediate that is immediately formulated, vialed, and cryopreserved to LCTOPC1 DP without the use of a hold step. Compositional analysis of LCTOPC1 by immunocytochemistry (ICC), flow cytometry, and quantitative polymerase chain reaction (qPCR) indicates that the cell population is comprised primarily of neural lineage cells of the oligodendrocyte progenitor phenotype. Other neural lineage cells, namely astrocytes and neurons, are present at low frequencies. The only non-neural cells detected in the population are epithelial cells. Mesodermal and endodermal lineage cells, and uhESCs are routinely below the quantitation or detection limits of the assays.

It is hypothesized that the subacute phase of SCI is the optimal time window in which to administer AST-OPC1. This phase avoids the early damage that leads to apoptosis of endogenous oligodendrocytes and occurs soon enough to allow the AST-OPC1 cells to migrate to denuded axons before extensive glial scarring has occurred. This hypothesis is supported by studies in rodent models of SCI that have shown functional benefits when AST-OPC1 or other similar preparations are injected 7 days after SCI, but no benefit if the interval between injury and injection is greater than 8 weeks (Keirstead 2005, Karimi-Abdolrezaee 2006). Since spontaneous functional recovery in rats with contusion SCI begins to plateau at about 6 weeks post-injury, injection of AST-OPC1 at 7 days corresponds to about ⅙ (17%) of the time elapsed between injury and the onset of recovery plateau.

The subacute phase of SCI is thought to be much longer in humans given that the rate of spontaneous recovery typically begins to plateau at about 6 months after SCI (Fawcett 2007). Extrapolating from the nonclinical efficacy data in rodents, injection of AST-OPC1 when one-sixth of the time to onset of recovery plateau has elapsed in humans would correspond to about 30 days post-injury.

The original dosing window of 14 to 30 days was selected to avoid the early hemorrhage and inflammation that occurs following SCI, as well as the scar tissue formation that occurs in the chronic phase of SCI. This window was also based on the preclinical data available prior to the initiation of this clinical study. However, a dedicated preclinical study was performed which suggested that the optimal dosing window in human subjects may extend to a maximum 60 days post-SCI.

Review of the new preclinical study data was conducted by a panel including study investigators, and several SCI experts to determine whether the dosing window should be adjusted. Additionally, during the review, consideration was given to the planned inclusion of subjects with a C4 NLI. The final recommendations indicated that a dosing window of 21 to 42 days post-SCI would still be considered within the subacute period, while allowing more time for subjects to be medically stable prior to undergoing elective surgery for AST-OPC1 injection. Therefore, AST-OPC1 was administered to subjects in this study at 21 to 42 days after SCI.

Tacrolimus management. Immunosuppression with tacrolimus was initiated between 6 and 12 hours after injection of AST-OPC1. If the subject was unable to take oral medication, tacrolimus was administered intravenously at a starting dose of 0.01 mg/kg/day, given as a continuous intravenous infusion. Subjects were switched to oral tacrolimus as soon as they were able to take medication by mouth. The starting dose for oral tacrolimus was 0.03 mg/kg/day, divided into 2 daily doses. The tacrolimus dose was adjusted to achieve a target whole blood trough level of 3 to 7 ng/ml. This target range was slightly below the typical range for long-term maintenance therapy following solid organ transplantation and was selected based on the low allogenic reactogenicity of AST-OPC1. At Day 46, the tacrolimus dose was decreased by 50% (rounded to the nearest 0.5 mg, since this was the smallest capsule size available). At Day 53, the tacrolimus dose was decreased by another 50% (rounded to the nearest 0.5 mg). If the rounded total daily dose was 0.5 mg or lower, the subject received 0.5 mg once per day until tacrolimus was discontinued. If the rounded total daily dose was 0.5 mg or lower, the participant received 0.5 mg once per day until tacrolimus was discontinued. Tacrolimus was discontinued at Day 60 (FIG. 2). The dose of tacrolimus was lowered if the trough blood level exceeded 7 ng/mL. In addition, an expert reviewed all ISNCSCI examination forms to assess whether there were any changes in neurological function that may have been associated with tacrolimus tapering and/or discontinuation. Tacrolimus was discontinued if any of the following occurred: infection, uncontrolled fever, liver function test elevation, serum creatinine elevation, seizure, or tacrolimus-induced thrombotic thrombocytopenia purpura. Tacrolimus was discontinued at Day 60.

A schedule of the evaluations and procedures that were to be performed from screening to day 365 is provided in Table 13 below.

TABLE 13
Schedule of Events
Part A

| | | | | Post-Injection | | |
|---|---|---|---|---|---|---|
| | Screen | Baseline | Surgery | | Day 7 | |
| | Days | Days | Injection | Days | (+/−1 | Days |
| Procedure | −11 to −3 | −2 to −1 | Day | 1-6 | day) | 8-29 |
| Demographic data | X | | | | | |
| Past and current | X | | | | | |
| medical history | | | | | | |
| Complete physical | X | | | | | |
| exam | | | | | | |
| Brief physical exam | | X | | Day 1 | X | |
| Vital signs | X | X³ | X | Daily | X | |
| Neurological exam | | X⁵ | | | X | |
| ISNCSCI exam | X | X¹ | | | X | |
| GRASSP | | X⁵ | | | | |
| SCIM | | X⁵ | | | | |
| MRI² | Day −7 | | | | X | |
| | to −3 | | | | | |
| ECG | X | | | | X | |
| Hematology | X | X | | Day 1 | X | |
| Blood chemistry | X | X | | Daily | X | 2/ |
| | | | | | | week |
| Serology for HIV, | X | | | | | |
| HBV, HCV | | | | | | |
| Panel reactive | X | | | | | |
| antibodies | | | | | | |
| Pregnancy test, if | X | | | | | |
| applicable | | | | | | |
| 48-hour blood | Day −3 | | | | | |
| culture | | | | | | |
| Fasting blood | | X⁴ | | | | |
| glucose | | | | | | |
| Blood for HLA | | X⁵ | | | | |
| typing | | | | | | |
| Blood for immune | | X⁵ | | | X | |
| response monitoring | | | | | | |
| Blood for | | X⁵ | | | | |
| xenotransplantation | | | | | | |
| archival | | | | | | |
| Withhold DVT | | Day −1 | | | | |
| prophylaxis | | | | | | |
| CSF via lumbar | | | X | | | |
| puncture | | | | | | |
| Begin tacrolimus | | | X | | | |
| Restart DVT | | | | Day 1 | | |
| prophylaxis | | | | | | |
| Tacrolimus blood | | | | Day 3⁶ | X | 2/ |
| levels | | | | | | week |

| | |
|---|---|
| Concomitant | [symbol] |
| medications | |
| Adverse events | |

Part B

| | | | | | Day | Day | |
|---|---|---|---|---|---|---|---|
| | Day 30 | | Day 60 | Day 90 | 180 | 270 | |
| | (+/−3 | Days | (+/−7 | (+/−7 | (+/−14 | (+/−14 | Day |
| Procedure | days) | 31-59 | days) | days) | days) | days) | 8-29 |
| Complete physical | | | | | | | X |
| exam | | | | | | | |
| Brief physical exam | X | | X | X | X | | |
| Vital signs | X | | X | X | X | | X |
| Neurological exam | X | | X | X | X | | X |
| ISNCSCI exam | X | | X | X | X | X | X |
| GRASSP | | | | X | X | X | X |
| SCIM questionnaire | X | | X | X | X | X | X |
| Pain questionnaire | X | | | X | X | | X |
| Bowel-Bladder | X | | | | X | | X |
| questionnaire | | | | | | | |
| MRI² | X | | | | X | | X |
| Hematology | X | | X | X | X | | X |
| Blood chemistry | X | 1/week | X | X | X | | X |
| Fasting blood | X | | | | | | |
| glucose | | | | | | | |
| Blood for immune | X | | X | X | X | | X |
| response monitoring | | | | | | | |
| Tacrolimus blood | X | 1/week | X | | | | |
| level | | | | | | | |
| CSF via lumbar | | | X | | | | |
| puncture | | | | | | | |
| Blood for | X | | | | | | X |
| xenotransplantation | | | | | | | |
| archival | | | | | | | |

| | |
|---|---|
| Concomitant | [symbol] |
| medications | |
| Adverse events | |

Abbreviations:
CSF, cerebrospinal fluid;
GRASSP, Graded Redefined Assessment of Strength, Sensibility and Prehension;
ISNCSCI, International Standards for Neurologic Classification of Spinal Cord Injury;
MRI, magnetic resonance imaging;
SCIM, Spinal Cord Independence Measure.
¹ISNCSCI exam (may be performed on Day −3 to −1, unless the sreening ISNCSCI was performed on Day −3)
²MRI of cervical spine and brain at Screen & Day 365; MRI of cervical spine only at Days 7, 30, 180
³Vital on day −2 and −1
⁴May be done pre-op on Injection Day
⁵May be performed as early as Day −4 if required to accommodate clinical site staff availability
⁶Tacrolimus blood level on Day 3 may be obtained +/−1 day.

Efficacy Evaluations. Neurological examinations were performed using the standardized International Standards for Neurological Classification of Spinal Cord Injury (ISNCSCI) examination for motor and sensory testing and for designation of the American Spinal Injury Association impairment scale. Upper Extremity Motor Score (0-50) and change from baseline were summarized with N, mean, standard deviation, 95% confidence interval, median, minimum, and maximum for the overall Intent to Treat population. A positive change is considered improvement and a negative change is considered worsening.

Motor and sensory level at each visit and changes in motor/sensory level from baseline were defined as follows. “Change in Level” is defined as the number of levels the motor/sensory level has changed from baseline. A positive number represents a change in the caudal direction; this is considered improvement. A negative number represents a change in the rostral direction; this is considered worsening.

The change in motor and sensory level was tabulated as follows: Percentage of subjects with an ascending motor level on either side of the body relative to baseline; Percentage of subjects with no motor level change on either side of the body relative to baseline; Percentage of subjects with one motor level improvement on at least one side of the body relative to baseline; Percentage of subjects with one motor level improvement on both sides of the body relative to baseline; Percentage of subjects with a two or more motor level improvement on at least one side of the body relative to baseline.

Efficacy Results. Efficacy for this study was measured by the change in ISNCSCI exam upper extremity motor score (UEMS) and change in motor level from baseline to 12 months after injection of AST-OPC1. A total of 22 subjects were part of the intent to treat population (Cohorts 2-5). The mean UEMS for the 22 subjects who completed the Day 365 visit was 28.4 (min: 7, max: 46), with a mean change from baseline of 8.9 points. There were three subjects (2004, 2007, 2008) who presented with some improvement in lower extremity motor scores (LEMS) without correlation with the level of improvement in UEMS. A total of 7 (31.8%) out of 22 subjects attained a two motor level improvement on at least one side of the body at the Day 365 visit and 21 (95.5%) subjects out of 22 subjects attained a one motor level improvement on at least one side at the Day 365 visit relative to Baseline. The percentage of subjects with motor level improvement is shown in Table 14 below.

TABLE 14
Percent of Motor Level Improvement from Baseline.

| | ≥2 Motor | ≥1 Motor | ≥1 Motor | | Ascending |
|---|---|---|---|---|---|
| | Levels At least | Level On both | Level At least | No change % | Motor Level % |
| Visit (N) | one side % (n) | sides % (n) | one side % (n) | (n) | (n) |
| Day 7 (22) | 0 (0) | 0 (0) | 45.5 (10) | 54.5 (12) | 0 (0) |
| Day 30 (22) | 9.1 (2) | 18.2 (4) | 45.5 (10) | 54.5 (12) | 0 (0) |
| Day 60 (21) | 14.3 (3) | 28.6 (6) | 61.9 (13) | 38.1 (8) | 0 (0) |
| Day 90 (22) | 18.2 (4) | 36.4 (8) | 81.8 (18) | 18.2 (4) | 0 (0) |
| Day 180 (22) | 13.6 (3) | 54.5 (12) | 86.4 (19) | 13.6 (3) | 0 (0) |
| Day 270 (20) | 25.0 (5) | 45.0 (9) | 85.0 (17) | 10.0 (2) | 5.0 (1) |
| Day 365 (22) | 31.8 (7) | 59.1 (13) | 95.5 (21) | 4.5 (1) | 4.5 (1) |

Study Endpoints. The primary endpoint of the trial was safety, as measured by the frequency and severity of adverse events (AEs) and serious adverse events (SAEs) within 1 year of LCTOPC1 injection that were related to LCTOPC1, the injection procedure used to administer LCTOPC1, and/or the concomitant immunosuppression administration. Measurements to assess safety included physical exams, vital signs, electrocardiogram (ECG), neurological exams, ISNCSCI exams, magnetic resonance imaging (MRI) scans, pain questionnaire, concomitant medications, AEs, and laboratory tests for hematology, blood chemistry, and immunosuppression safety monitoring. The secondary endpoint was neurological function as measured by the International Standard for Classification of Spinal Cord Injury (ISNCSCI). The ISNCSCI is a highly reproducible research and clinical assessment of neurological impairment for individuals with SCI and has been used as a tool to evaluate the effectiveness of acute SCI clinical interventions (Rupp PMID: 34108832; Marino 2008 PMID: 18581663). To maximize the inter-rater and intra-rater reliability of neurological assessments, a half-day training session, led by an external expert and including examinations of individuals with SCI, was required as part of each center's site initiation visit. During the first year of the study, ISNCSCI examinations were performed at 30, 60, 90, 180, 270, and 365 days after injection of LCTOPC1. The 365-day follow-up visit was pre-specified as the time point for the secondary endpoint.

STATISTICAL ANALYSIS FOR EFFICACY. The efficacy endpoint, neurological function, was evaluated by characterizing upper extremity motor scores and motor level on the ISNCSCI examination (point estimate and 95% confidence interval) by time point at 30, 60, 90, 180, 270, and 365-days post-injection of LCTOPC1. The baseline for the ISNCSCI assessment was defined as the Baseline Visit performed between 24 to 48 hours prior to injection. Upper Extremity Motor Score and change from baseline was summarized by participant, mean, standard deviation, 95% confidence interval, median, minimum, and maximum for the overall intent-to-treat population.

STATISTICAL ANALYSIS FOR SAFETY. The collection period for adverse events (AEs) began once the participant had signed the informed consent form and ended after 365 days of observation. Statistical analysis of AEs started on or after the date and time of the LCTOPC1 injection, or an AE that started before the LCTOPC1 injection, and worsened after the administration of the investigational product. AEs were tabulated by system organ class (SOC) and by preferred term (PT) within system organ class, according to the Medical Dictionary for Regulatory Activities (MedDRA®) Version 18 and reported by participants. A topline summary of AEs with the number of events, number of participants, and percentage of participants for each category was tabulated by cohort and overall. Categories for possible relationship included: LCTOPC1, injection procedure, and tacrolimus. Tabulations were prepared for all AEs, related events, Grade 3 and higher events, and serious events.

TABLE 15
Summary of Related Adverse Events in All Treated Participants

| | | | | Days | | |
|---|---|---|---|---|---|---|
| | AE | SAE | | from | | |
| Preferred Term | (N) | (N) | Causality | Injection | Outcome | Severity |
| Dysesthesia | 1 | None | OPC1 | 47 | Resolved* | Grade 2 |
| Cerebrospinal | 2 | 1 | Injection | 11-15 | Resolved | Grade |
| Fluid Leakage | | | procedure | | with | 2-3 |
| | | | | | sequelae** | |
| Cerebrospinal | 5 | None | Injection | 6-7 | Resolved | Grade 1 |
| fluid retention | | | procedure | | | |
| Pain (neck, | 5 | None | Injection | 0-1 | Resolved | Grade |
| muscular, | | | procedure | | | 1-3 |
| incisional) | | | | | | |
| Headache | 3 | None | Injection | 0-84 | Resolved | Grade |
| | | | procedure | | | 1-2 |
| Nausea | 1 | None | Injection | 0 | Resolved | Grade 2 |
| | | | procedure | | | |
| Postoperative | 1 | None | Injection | 7 | Resolved | Grade 1 |
| wound infection | | | procedure | | | |
| Vomiting | 1 | None | Injection | 0 | Resolved | Grade 2 |
| | | | procedure | | | |
| Autonomic | 1 | None | Injection | 0 | Resolved | Grade 3 |
| Dysreflexia | | | procedure | | | |
| Incisional | 1 | None | Injection | 1 | Resolved | Grade 1 |
| drainage | | | procedure | | | |
| Hypo- | 8 | None | Tacro- | 5-43 | Resolved | Grade |
| magnesemia | | | limus | | | 1-2 |
| Bacterial | 1 | 1 | Tacro- | 30 | Resolved | Grade 3 |
| infection | | | limus | | | |
| Decreased | 1 | None | Tacro- | NK | Resolved | Grade 2 |
| Appetite | | | limus | | | |
| Nausea | 1 | None | Tacro- | 7 | Resolved | Grade 2 |
| | | | limus | | | |

Abbreviations:
NK, not known;
AE, adverse event;
SAE, serious adverse event.
*Dysesthesia resolved at Year 2 follow-up.
**One Cerebrospinal Fluid Leakage required lumbar drainage. Participant returned to Rehabilitation on Day 22.

SUMMARY OF CERVICAL TRIAL. The overall safety profile of OPC1 was excellent, and immunosuppression with tacrolimus was well-tolerated. MRI scans were consistent with a very high rate (96%) of durable engraftment through 1 year post-injection. The majority of participants who received 10M or 20M OPC1 cells exhibited motor recovery in the upper extremities. 21/22 participants in cohorts 2-5 improved at least 1 motor level on at least 1 side. 7/22 participants in cohorts 2-5 improved at least 2 motor level on at least 1 side. Two issues (C4 NLI; postop cord compression) that may negatively impact motor recovery are believed to be addressable in future studies. These encouraging engraftment and motor recovery data warrant further evaluation in studies incorporating a period of rehabilitation utilizing novel strategies designed to augment the potential of hESC base therapies to promote functional recovery. Data from the Cervical Trial will help inform the design of future randomized studies with respect to inclusion/exclusion criteria, dose, and timing of administration.

DISCUSSION The safety data from this study suggest that AST-OPC1 can be safely administered to participants in the subacute period after cervical SCI. The injection procedure and the low-dose temporary immunosuppression regimen were well tolerated. None of the 25 participants who received LCTOPC1 showed evidence of neurological deterioration. There were no SAEs reported as directly related to LCTOPC1 and evaluation of the AEs did not show an increase in incidence for commonly reported SCI complications, such as urinary tract infections, muscle spasms, or neuropathic pain (Sezer 2015). In this study involving participants with cervical C4-C7 AIS-A and AIS-B, at one-year follow-up, 24/25 (96%) of participants recovered one or more levels of motor function on at least one side of their body and 8/25 (32%) of participants recovered two or more levels of motor function on at least one side of their body. Improvement of two motor levels can change a person's functional capacity from requiring total assistance for activities of daily living to near independence (Whiteneck et al. 1999). The safety and neurological recovery data, from both the thoracic and cervical trials, have provided evidence that hESC-derived treatments can be safely delivered into the spinal cord.

Overall, there were several improvements in production and quality of OPC1 following the first-in-man study. A new ready-to-inject formulation was developed, elimination of dose preparation was achieved, there was a 10- to 20-fold increase in production scale, there was significant reduction in product impurities, there were improvements in functional activity, 12 new analytical and functional methods were developed, and all animal-based production reagents were eliminated.

### Example 10—Decorin Secretion as a Potency Assay for OPC1

Treatment with recombinant Decorin in rat models of spinal cord injuries (SCI) has been shown to inhibit inflammation and glial scar formation and may promote axonal growth across the injury interface after acute spinal cord injury (Wu, Li et al, 2013, Ahmed, Bansal et al. 2014). Decorin has been shown to suppress acute scarring and wound cavitation and induce dissolution of mature scar tissue in dorsal funicular lesion SCI model system of the spinal cord in adult rats (Wu, Li et al, 2013, Ahmed, Bansal et al 2014). DFL cavity treatment with recombinant Decorin suppresses inflammation and scar deposition in the acute and subacute phases of the CNS injury response in rat model of SCI and also contributes to dissolution of the mature scar following SCI (Esmaeli, Berry et al 2014, Ahmed, Bansal et al 2014). OPC1 treatment in non-clinical models of SCI has demonstrated similar results to that seen in the published studies above.

OPC1 cells have been shown to produce large amounts of Decorin. The results seen in the OPC1 animal studies demonstrate very similar anatomical outcomes to that seen in the studies above. Thus, the anatomical effects observed in the nonclinical efficacy studies of OPC1 transplantation into SCI injury may be attributed, at least in part, to the secretion of Decorin.

While the preclinical studies to date have established the optimal window for OPC1 implantation to achieve maximum efficacy between 14-60 days post-injury, Decorin has been shown to have an effect in dissolving mature scars following SCI (Esmaeli, Berry et al 2014, Ahmed, Bansal et al 2014), lending evidence for a potential role of OPC1 cells in the treatment of chronic SCI subjects.

### Introduction

Decorin is a naturally occurring extracellular small leucine-rich proteoglycan TGF-β1/2 antagonist which regulates diverse cellular functions through interactions with components of the extracellular matrix (ECM) and plays several key roles in the cellular response to spinal cord injury. Accordingly, Decorin secretion in vitro was developed and qualified as a potency assay for OPC1

Briefly, OPC1 Drug Product cells are thawed and cultured for 48 hours, then the media is collected and secreted Decorin concentration measured by an ELISA assay.

Preliminary molecular analysis and an initial ELISA revealed that Decorin is not secreted by H1 hESC, and that its expression is gradually turned on during OPC1 differentiation, with the highest levels of a secreted protein detected in the drug product. This, along with scientific literature showing a biological activity inclusive of scarring suppression at injury site and stimulation of axonal growth through spinal cord injury (SCI) site, supports Decorin as a suitable potency candidate.

Decorin (secretion) is useful as a potency indicator for OPC1 cells by describing its ability to modulate SCI tissue remodeling, via attenuating harmful processes.

### Decorin is a Biological Modulator Secreted by OPC1

OPC1 Drug Product is a cryopreserved cell population containing oligodendrocyte progenitor cells and other characterized cell types that are obtained following differentiation of H1 human embryonic stem cells (hESC). OPC1 has been shown to have three potentially reparative functions which address the complex pathologies observed at the SCI injury site. These activities of OPC1 include production of neurotrophic factors, stimulation of vascularization, and induction of remyelination of denuded axons, all of which are critical for survival, regrowth and conduction of nerve impulses through axons at the injury site. One of the potential routes by which OPC overcome the inhibitory factors at the injury site may be Decorin upregulation as a response of NG2″ (neuron-glial antigen 2, CSPG4) cells to retinoic acid (Goncalves, Wu et al. 2019).

It is important to note that Decorin secretion is acquired by OPC1 cells during the differentiation process as part of their maturation, feature enabling easy and quantifiable Decorin measurement as a possible potency marker of OPC1.

Decorin secretion was approximately 25 ng Decorin per day. Accordingly, the current threshold for the Decorin potency assay was defined as 25 ng/ml.

Decorin has been shown to be effective in reducing scarring, when produced endogenously by different cell types at the injury site or given exogenously in a recombinant form in non-clinical models of SCI, as described below.

Thus, active de-novo secretion of endogenous Decorin by OPC1 during its implantation into the SCI cavity may be a key component to ensure successful treatment, and, as such, may be a good potency marker.

### Spinal Cord Injury

The majority of traumatic SCIs result in contusion or compression of the spinal cord. The mechanical insult (primary injury) in these cases causes a cascade of molecular and cellular changes that are collectively referred to as the secondary injury (Kakulas 1999). Some of the pathological changes associated with secondary injury include petechial hemorrhages progressing to hemorrhagic necrosis, free radical-induced lipid peroxidation, elevated intracellular calcium leading to activation of neutral proteases, accumulation of extracellular potassium, accumulation of excitatory amino acids, and ischemia (Anderson 1993, Hulsebosch 2002). Traumatic demyelination also begins within a few hours after injury (Kakulas 1999).

The cellular response to SCI is generally considered to consist of 3 phases: an acute hemorrhagic phase when hematogenous inflammatory cells invade the wound; a sub-acute phase when scarring is initiated from astrocytes interacting with invading meningeal fibroblasts to produce a glia limitans around the wound cavity with a core of extracellular matrix (ECM) proteins, revascularization is also initiated, and axon growth is arrested at the wound margins; and a consolidation or chronic phase when ECM deposits are remodeled by proteases to establish a mature contracted scar.

The superimposition of progressive wound cavitation on top of the cellular response results in a progressive cystic expansion of an astrocyte-free void filled with proteoglycans and macrophages and bordered by a proteoglycan-rich neurophils that cause secondary destruction of axons.

### Cellular and Extracellular Composition of the Spinal Cord Injury Scar Tissue

Traumatic SCI triggers a complex cascade of events that culminates in the formation of a scar which consists of multiple cell types, as well as extracellular and non-neural components. In the acute post-injury phase (0-72 h), cell death and damage lead to release of a number of cellular and blood-derived damage associated molecular patterns (DAMPs). These are powerful activating and inflammatory stimuli for stromal cells, astrocytes, NG2+ OPCs and microglia. Fibroblast-like cells proliferate from perivascular origin in this acute phase. Activated cells increase deposition of ECM molecules such as Chondroitin sulfate proteoglycans (CSPGs) and stromal-derived matrix. Circulating immune-responders (neutrophils, monocytes) are recruited, their relative expression of cytokines, chemokines and matrix metalloproteinases is that of a mixed cell phenotype (pro-inflammatory and pro-resolving). Over time, the injury microenvironment becomes increasingly proinflammatory. In the chronic spinal injury scar, monocyte-derived macrophages/microglia adopt a predominantly pro-inflammatory phenotype. Rather than entering a resolution phase, responding innate immune cells present DAMPs to circulating adaptive immune cells and the pathology spreads. Hypertrophy of reactive astrocytes, upregulated expression of intermediate-filament associated proteins and secretion of matrix CSPGs occur. Scar-forming reactive astrocytes are organized into a barrier-like structure, which separates spared tissue from a central region of inflammation and fibrosis where wound-healing fails to undergo resolution. In most mammalian species, a chronic cystic cavity develops. Wallerian degeneration of injured axonal projections contributes to continued extracellular deposition of axonal and myelin debris, which is ineffectively processed by immune cells, and along with CSPGs, acts to inhibit neuronal regeneration and neuroplasticity (Bradbury and Burnside 2019).

### Function of Decorin in Spinal Cord Injury

OPC1 clinical application is aimed at the sub-acute phase, 21-60 days post-SCI. It is thus assumed that the transplantation of OPC1 occurs during the transition from acute to chronic phase, in an inflammatory active environment. Hence, the ability of OPC1 to actively secrete Decorin that can potentially reduce the ongoing negative cues may be useful for its therapeutic activity.

Decorin suppresses CNS scarring through several mechanisms (Esmaeili, Berry et al. 2014, Gubbiotti, Vallet et al. 2016) including: (1) attenuating both TGF-β1/2 receptor activation and signaling through down-stream SMADs (a family of intracellular proteins that mediate signaling by members of the TGF-beta superfamily), that mediate transcriptional activation of ECM production: (2) binding to type I collagen fibrils to inhibit fibrogenesis; (3) forming an activity-blocking complex with connective tissue growth factor (CTGF); (4) binding to fibronectin and inhibiting cell adhesion and fibroblast migration; (5) abrogating inflammation, CSPG/laminin/fibronectin-rich scar formation and the injury responses of astrocytes, microglia and macrophages; (6) stimulating microglia to secrete plasminogen/plasmin (the activity of which is moderated by PAI-1), which then regulates matrix metalloproteinase (MMP): tissue inhibitors of MMP (TIMP) ratios in wounds to initiate fibrolytic degradation of ECM underpinning remodeling during the consolidation phase of acute scarring; and (7) binding to the epidermal growth factor receptor (EGFR), hepatocyte growth factor (Met) receptor and toll-like receptors to modulate angiogenesis and inflammatory responses.

Recombinant human Decorin (Galacorin), was investigated as a potential treatment for macular degeneration, diabetic retinopathy and diabetic macular edema (Nastase, Janicova et al. 2018). In the patent U.S. Pat. No. 9,061,047B2, the authors suggest using Galacorin for preventing or reducing scar formation by its administration to patients with neurological conditions including central nervous system injuries and/or diseases. A formulation of Decorin in an eye drop was reported as an anti-scarring agent that can replace corneal transplantation (Hill, Moakes et al. 2018).

Decorin promotes axon regeneration directly by suppressing the production of scar-derived growth inhibitory ligands (Esmaeili, Berry et al. 2014) and indirectly by: (1) plasmin activation of neurotrophins (Davies, Tang et al. 2006); (2) disinhibition of axon growth cone advance by digestion of CSPG and CNS myelin inhibitors through plasmin and plasmin induced activation of MMP (Minor, Tang et al. 2008); and (3) suppression of EGFR activity in growth cones, thereby potentially blocking CSPG/CNS myelin mediated growth cone collapse.

Treatment with Recombinant Decorin in Rat Models of SCI Supports Therapeutic Potential

Recombinant human Decorin (rh-Decorin) has been shown to inhibit inflammation, glial scar formation and CSPG expression, and may promote axonal growth across the injury interface after acute spinal cord injury (Wu, Li et al. 2013, Ahmed, Bansal et al. 2014). These data show that Decorin treatment in animal models commenced immediately after spinal cord injury, inhibits TGF-β1/2-mediated invasion of inflammatory cells, scar deposition and cavitation and that later, during the consolidation phase, regulates ECM remodeling by both the induction of MMP and tissue plasminogen activator (tPA) activity and suppression of TIMP and PAI-1. Moreover, in Decorin-treated mature scars in which acute titers of TGF-β1/2 have declined, scar dissolution appears to be induced by MMP/tPA mediated fibrolytic activities and enhanced by depressed levels of TIMP and PAI-1 activity.

Decorin suppressed acute scarring (fibrogenesis) and wound cavitation, and induced dissolution of mature scar tissue (fibrolysis) in dorsal funicular lesion (DFL) SCI model system of the spinal cord in adult rats (Wu, Li et al. 2013, Ahmed, Bansal et al. 2014). DFL cavity treatment with recombinant Decorin suppresses inflammation and scar deposition in the acute and subacute phases of the CNS injury response in rat model of SCI and also contributes to dissolution of the mature scar following SCI. Decorin treatment of spinal cord injury (Esmaeili, Berry et al. 2014, Ahmed, Bansal et al. 2014).

Additionally, Decorin promoted axonal regrowth in both acute and chronic experiments. In both cases, axons were absent in PBS-treated DFL, but present in Decorin-treated DFL.

OPC1 Treatment in Non-Clinical Models of SCI Demonstrates Similar Results to that Seen with Decorin Treatment

In five studies (Table 16) of OPC1 transplant into rodent models of SCI, a statistically significant reduction of cavitation area was observed in OPC1-treated animals, as compared to animals injected with control vehicle (HBSS or IsoLyte plus Human Serum Albumin). In these studies, axonal regrowth through the SCI lesion was seen in all OPC1-treated animals but not in the control animals. Tabulated results of these studies are shown below. The rat model of SCI injury that was used closely emulates the damage and outcomes seen in human after a contusion or crush injury of the cervical spinal cord. For rat model treatment time windows after SCI injury are defined as: Day 1-7: acute stage; Day 7-14: subacute stage; and over 14 days: chronic. In other examples, the route of administration can include transplating to the subject the composition (e.g., including OPC1) using a PSD system, for example the PSD system as described herein. In other examples, the route of administration can include transplating to the subject the composition (e.g., including OPC1) using the PSD shown and described in U.S. Publication No: 2015/0224331, incorporated by reference herein in its entirety.

TABLE 16
Summary of preclinical studies demonstrating similar results seen
with Decorin treatment

| Study | Timepoint | Study Type | Results |
|---|---|---|---|
| P002-2011 (Seq. 0073) | 4 months | Efficacy study in rats (treated 7- | 86% Reduction in |
| | | 3 days after spinal cord | cavitation area over |
| | | contusion) | control |
| 1058-015 (Seq. 0073) | 9 months | Tox study in rats (treated 6-8 | 4% of OPC1 treated |
| | | days after spinal cord | and 63% of controls |
| | | contusion) | had measurable areas |
| | | | of cavitation |
| 1058-021 (Seq. 0073) | 9 months | Tox study in rats (treated 6-8 | 4% of OPC1 treated |
| | | days after spinal cord | and 58% of controls |
| | | contusion) | had measurable areas |
| | | | of cavitation |
| 2237-001, (Seq. 0100) | 9 months | Tox study in rats (treated 6-8 | 5% of OPC1 treated |
| | | days after spinal cord | and 84% of control |
| | | contusion) | had measurable areas |
| | | | of cavitation |
| SP01-2016 (Seq. 0094) | 2 months | Study comparing timing of | 4-5 fold reduction of |
| | | transplantation (1 week, 2 | cavitation for OPC1 |
| | | weeks, 3 weeks) (treated 2-3 | treated over control at |
| | | weeks after spinal cord | 1 and 2 weeks, little |
| | | contusion) | cavitation reduction |
| | | | for OPC1 treated at 3 |
| | | | weeks over control |

Staining for the presence of myelinated axonal fibers performed in the first four studies listed above has shown the presence of myelinated axonal fibers traversing the lesion area in animals treated with OPC1 but not in the animal treated with the control.

The results observed in the OPC1 animal studies demonstrate very similar anatomical outcomes to that seen in the studies where Decorin infused implants were transplanted into animals with spinal cord injuries as described above (Wu, Li et al. 2013, Ahmed, Bansal et al. 2014). Thus, the anatomical effects observed in the nonclinical efficacy studies of OPC1 transplantation into SCI injury may be attributed, at least in part, to the secretion of Decorin.

### Summary

The current knowledge, as presented above, on the positive role that Decorin plays in attenuating the damage in the SCI cavity and the nonclinical studies results in Decorin-treated and OPC1-treated models of SCI justifies its utilization as a potency indicator for OPC1. It is projected that the ability of OPC1 cells to produce and secrete Decorin is one of the key therapeutic effects of OPC1, by positively modulating scarring and axon regrowth inhibiting processes. Therefore, we intend to use a qualified Decorin assay to be included in the panel of release testing for the new, improved OPC1 production process.

### References for Example 10

Ahmed, Z., D. Bansal, K. Tizzard, S. Surey, M. Esmaeili, A. M. Gonzalez, M. Berry and A. Logan (2014). “Decorin blocks scarring and cystic cavitation in acute and induces scar dissolution in chronic spinal cord wounds.” Neurobiol Dis 64:163-176.
Bradbury, E. J. and E. R. Burnside (2019). “Moving beyond the glial scar for spinal cord repair.” Nat Commun 10 (1): 3879.
Davies, J. E., X. Tang, J. C. Bournat and S. J. Davies (2006). “Decorin promotes plasminogen/plasmin expression within acute spinal cord injuries and by adult microglia in vitro.” J Neurotrauma 23 (3-4): 397-408.
Esmaeili, M., M. Berry, A. Logan and Z. Ahmed (2014). “Decorin treatment of spinal cord injury.” Neural Regen Res 9 (18): 1653-1656.
Goncalves, M. B., Y. Wu, E. Clarke, J. Grist, C. Hobbs, D. Trigo, J. Jack and J. P. T. Corcoran (2019). “Regulation of Myelination by Exosome Associated Retinoic Acid Release from NG2-Positive Cells.” J Neurosci 39 (16): 3013-3027.
Gubbiotti, M. A., S. D. Vallet, S. Ricard-Blum and R. V. lozzo (2016). “Decorin interacting network: A comprehensive analysis of decorin-binding partners and their versatile functions.” Matrix Biol 55:7-21.
Hill, L. J., R. J. A. Moakes, C. Vareechon, G. Butt, A. Ng, K. Brock, G. Chouhan, R. C. Vincent, S. Abbondante, R. L. Williams, N. M. Barnes, E. Pearlman, G. R. Wallace, S. Rauz, A. Logan and L. M. Grover (2018). “Sustained release of decorin to the surface of the eye enables scarless corneal regeneration.” NPJ Regen Med 3:23.
Minor, K., X. Tang, G. Kahrilas, S. J. Archibald, J. E. Davies and S. J. Davies (2008). “Decorin promotes robust axon growth on inhibitory CSPGs and myelin via a direct effect on neurons.” Neurobiol Dis 32 (1): 88-95.
Nastase, M. V., A. Janicova, H. Roedig, L. T. Hsieh, M. Wygrecka and L. Schaefer (2018). “Small Leucine-Rich Proteoglycans in Renal Inflammation: Two Sides of the Coin.” J Histochem Cytochem 66 (4): 261-272.
Wu, L., J. Li, L. Chen, H. Zhang, L. Yuan and S. J. Davies (2013). “Combined transplantation of GDAs (BMP) and hr-decorin in spinal cord contusion repair.” Neural Regen Res 8 (24): 2236-2248.

### Example 11—Comparability of GPOR OPC1 Cells and LTCOPC1 OPC1 Cells

Some of the process for making the cells and the OPC1 product, such as some of those used in the clinical studies described in the Examples herein, are referred to as Geron process and Geron cells or GPOR. The new processes described in this and other Examples herein are used for making the LCTOPC1 product. The LCTOPC1 process described herein is the process used for current GMP production of cells for clinical use. Data will be provided in this Example, supporting comparability between these manufacturing processes.

### List of Abbreviations

ASIA American Spinal Injury Association Impairment
bFGF Basic FGF
BMP Bone Morphogenic Protein
CMC Chemistry Manufacturing Controls
CS10 CryoStor 10
DP Drug Product
ECM Extracellular Matrix
FCM Flow Cytometry
GMP Good manufacturing Process
GPM Glial Progenitor Medium
GPOR Geron Process of Records
hESC Human Embryonic Stem Cells
hsFGF Heat-Stable FGF
IPC In-process Controls
MCB Master Cell Bank
NB Neurobody
NLI Neurological Level of Injury
OCB Original Cell Bank
OL Oligodendrocytes
POC Proof-of-Concept
QC Quality Control
RA Retinoic Acid
R&D Research and Develoment
rhEGF Recombinant Human EGF
RL Risk Level
RMAT Regenerative Medicine Advanced Therapy
SCI Spinal Cord Injury
TAI Thaw-and-Inject
UEMS Upper Extremity Motor Score
WCB Working Cell Bank

LCTOPC1 (OPC1), previously referred to as GRNOPC1 and then AST-OPC1, is an oligodendrocyte progenitor cell population derived from the H1 hESC line intended for one-time administration for the treatment of subacute spinal cord injury (SCI). OPC1 has been shown in pre-clinical studies to produce neurotrophic factors, migrate in the spinal cord parenchyma, stimulate vascularization, and induce remyelination of denuded axons, all of which are essential functions of oligodendrocyte progenitors and are important for survival, regrowth and function of axons.

Clinical evaluation of LCTOPC1 was initiated in 2010 by Geron Corporation. The first clinical trial was a Phase 1 safety study (NCT01217008) in which a low dose of 2×10⁶ OPC1 cells was injected into the lesion site of subjects with subacute, neurologically complete thoracic spinal T3-T11 injuries. A total of 5 subjects out of the planned 8 received OPC1 as part of the original Phase 1 CP35A007 safety study from October 2010 through November 2011.

In 2014, a Phase 1/2a study (NCT02302157) dose escalation of OPC1 in subjects with subacute sensorimotor complete (American Spinal Injury Association Impairment Scale A (ASIA Impairment Scale A)), Single Neurological Level (SNL) from C5 to C7 cervical spinal cord injuries was initiated, with the later expansion of the study to patients with a C4 Neurological Level of Injury (NLI) if the Upper Extremity Motor Score (UEMS)≥1 and changing the dosing window from 14-30 days to 21-42 days post-spinal cord injury. A total of 25 subjects across 5 cohorts were enrolled in the AST-OPC-01 study and received a single administration of OPC1 cells delivered by intra-parenchymal injection into the spinal cord injury site using a Syringe Positioning Device, during a dedicated surgical procedure. The enrollment for AST-OPC-01 study was completed in December 2017 and reported in December 2020.

Briefly, the origin of the new Master Cell Bank (MCB) is the H1 Bank Lot. No. MCBH101. MCBH101 was manufactured by Geron directly from the H1 Original Cell Bank (OCB) in 2009. It was manufactured in feeder-free conditions using well-defined raw materials, new culturing system and harvesting procedure, and cryopreserved by an hESC-customized cryopreservation process. In addition, the method for assessment of H1 hESC pluripotency was optimized. The new WCB originated from the new MCB and was expanded in tissue culture for 4 passages, while maintaining hESC characteristics, and then cryopreserved. The WCB will provide the starting material for LCTOPC1 manufacturing.

The purpose of this Example is also to present the scientific data generated during the development of LCTOPC1 CMC program. The provided information includes the development plans for LCTOPC1 with regards to preliminary comparability results based on R&D runs of the improved manufacturing process, comparability between the GPOR and LCT R&D manufactured material, introduction of a new proposed potency assay, review of the OPC1 safety status based on the GPOR in vivo data and reanalysis of GPOR lots, utilizing improved analytical methods.

### Rationale for Process Improvements

OPC1 is an investigational drug studied in a Phase 1 and a Phase 1/2a spinal injury clinical studies using OPC1 clinical lots produced by Geron Inc. Geron's (GPOR) manufacturing process was originally developed in the early 2000s. At that time, well-defined and cell therapy grade reagents and materials were not widely available. As such, Stage 1 of the manufacturing process included the propagation of H1 embryonic cells on Matrigel™, an animal derived Extracellular Matrix (ECM), collagenase, and manual scraping of the culture dish surface for harvesting, passaging and expansion of the H1 embryonic stem cells.

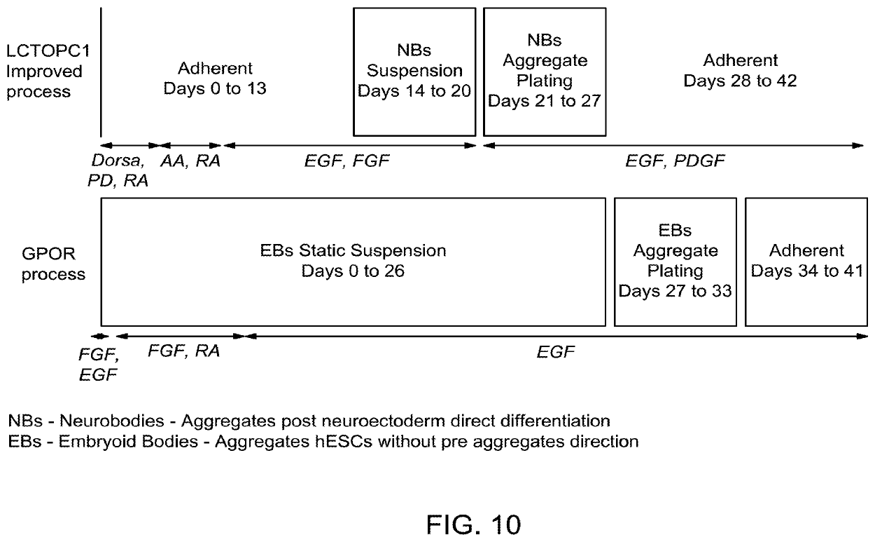

Furthermore, the GPOR manufacturing process was based on a poorly controlled differentiation process, driven by three guiding molecules. Most of the differentiation process occurred in cell aggregates starting directly from pluripotent H1 cells, in the form of Embryoid bodies (day 0 to day 26, FIG. 10), which have a strong susceptible to spontaneous differentiation. From day 27 on, the differentiation was completed on Matrigel™ coated adherent surface for oligodendrocyte progenitor expansion and maturation. The GPOR manufacturing process had low yields, and key quality attributes defined by purity/impurity/non-targeted cell population markers exhibited limited reproducibility. Additionally, the final cryopreserved product required upon thawing, washing and formulation preparation prior to administration.

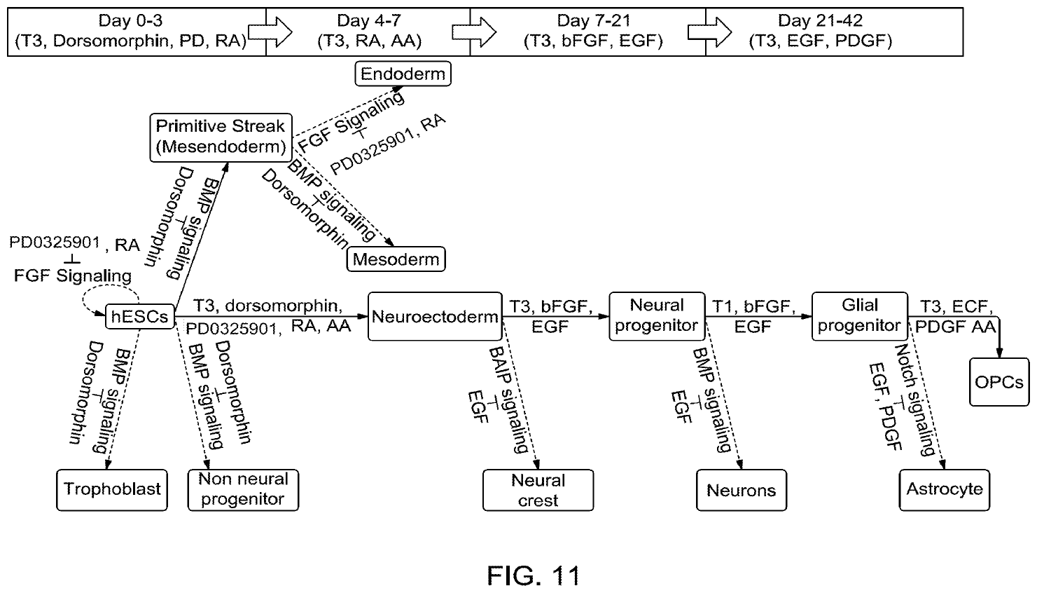

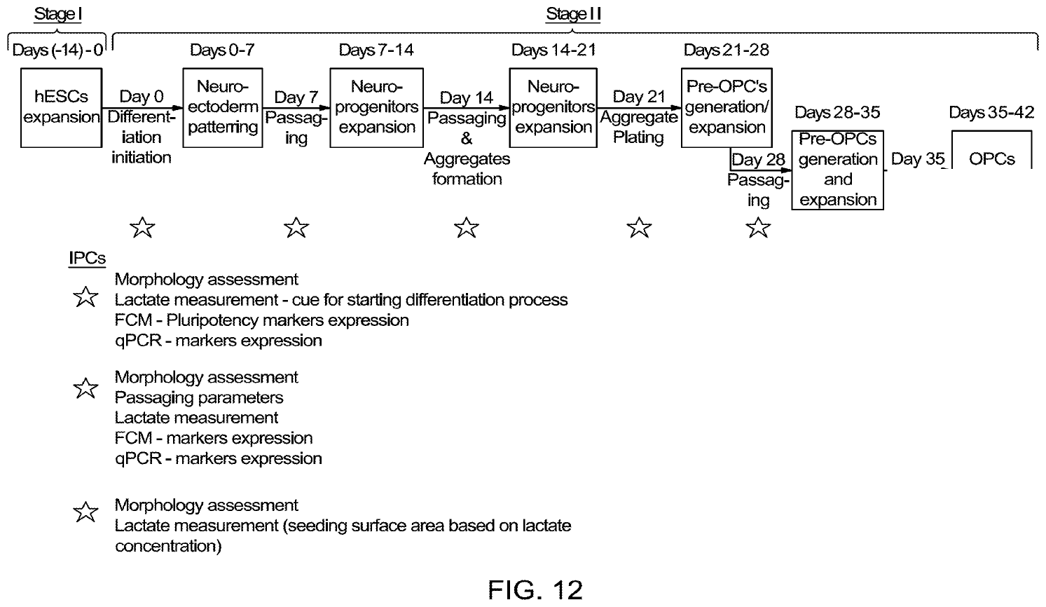

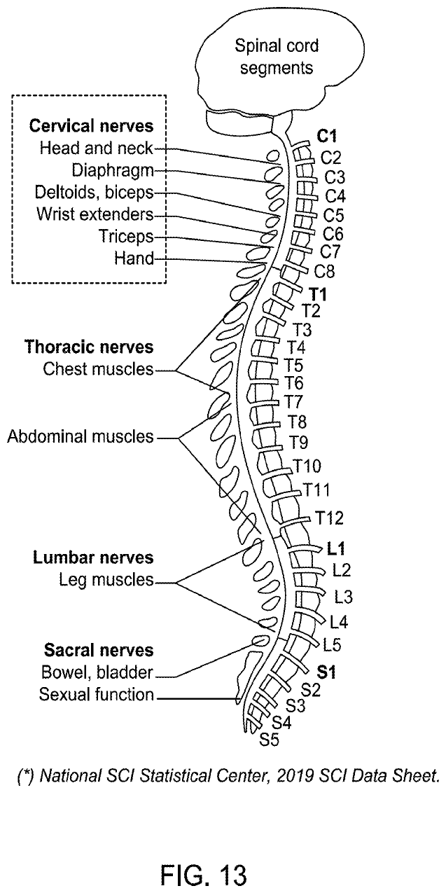

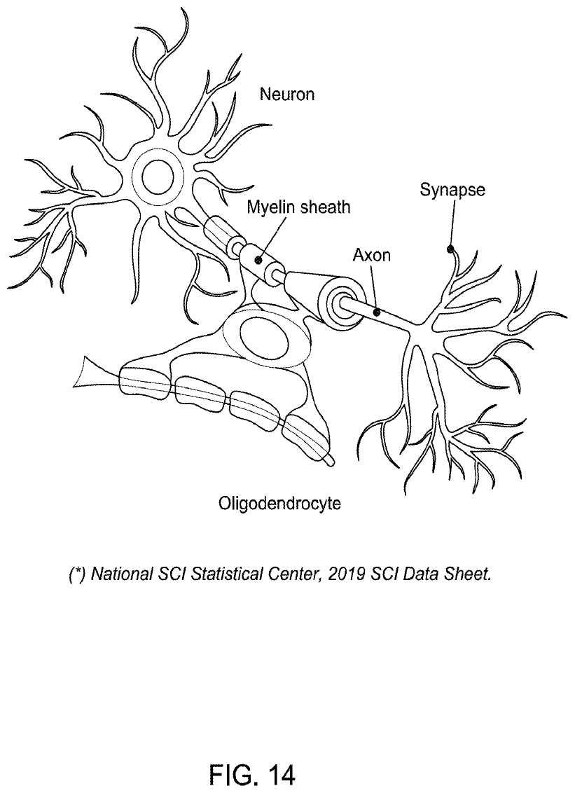

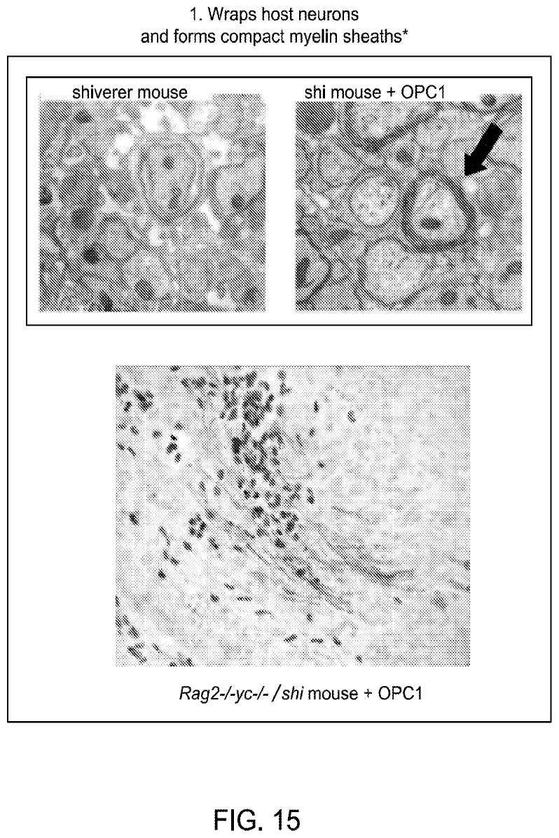

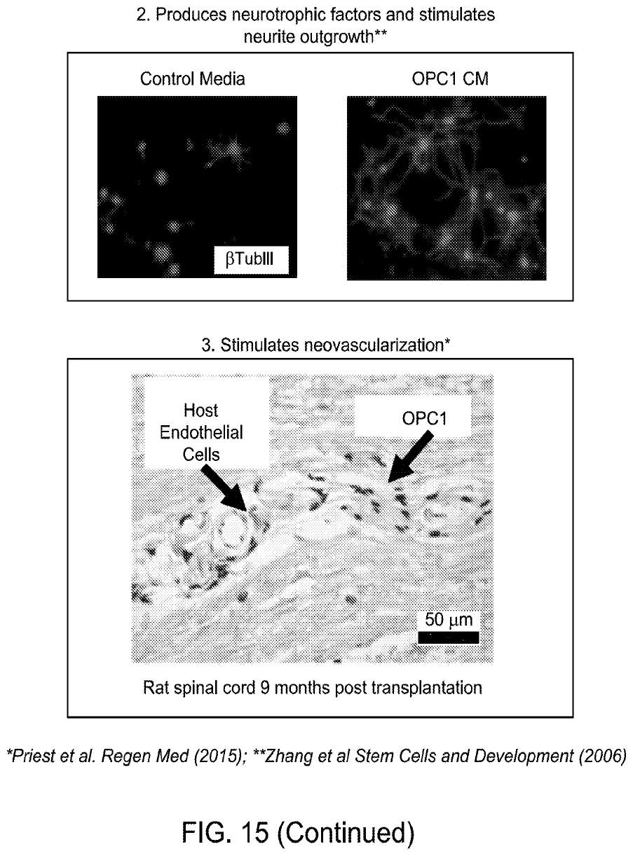

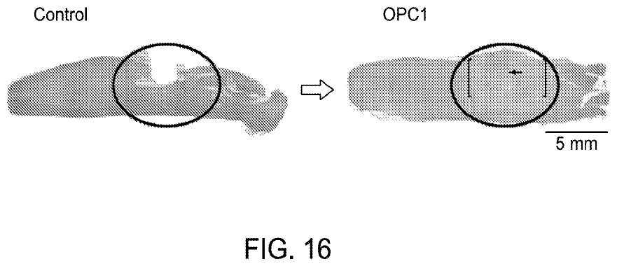

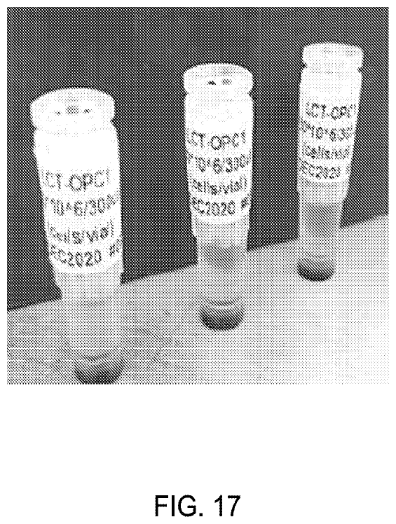

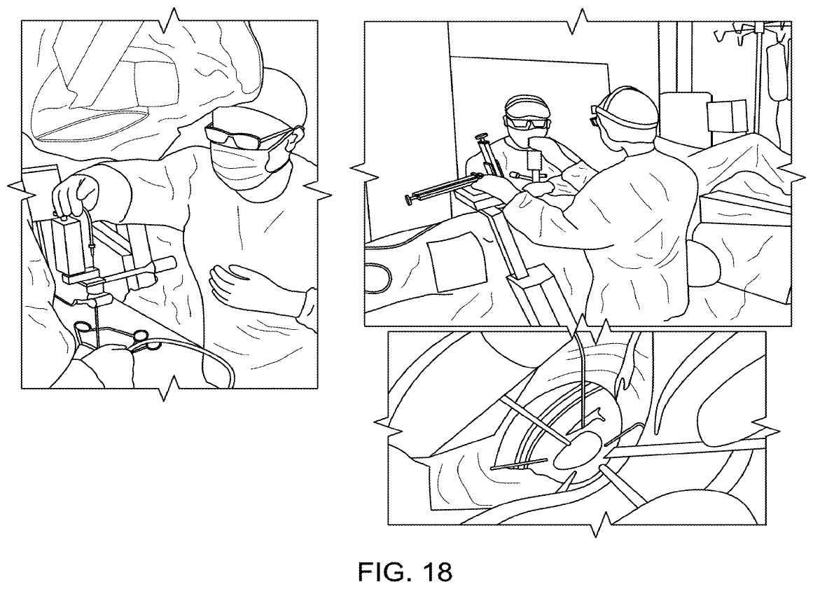

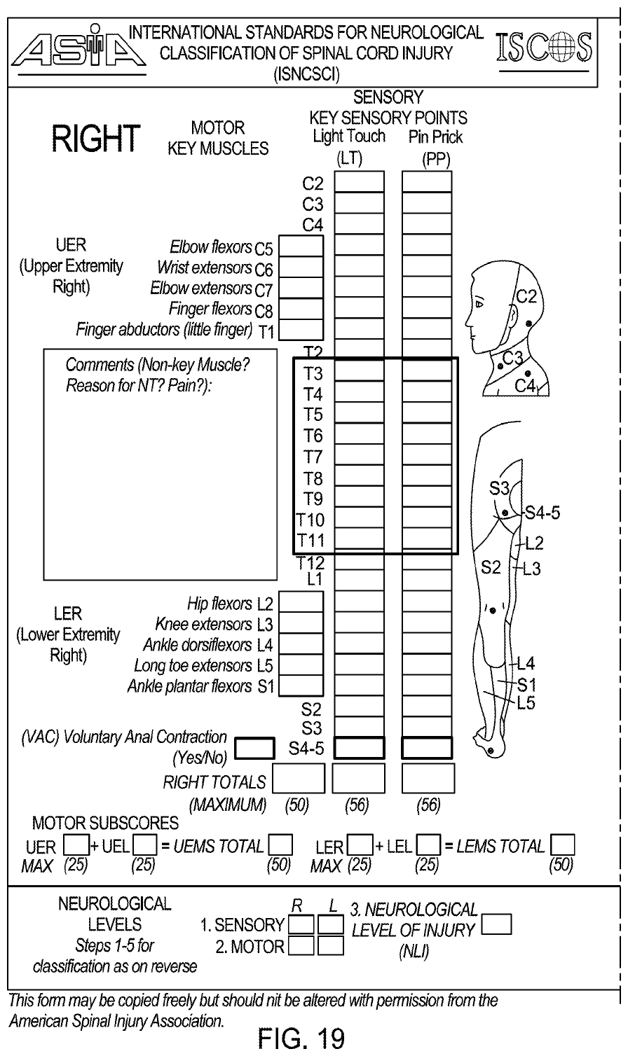

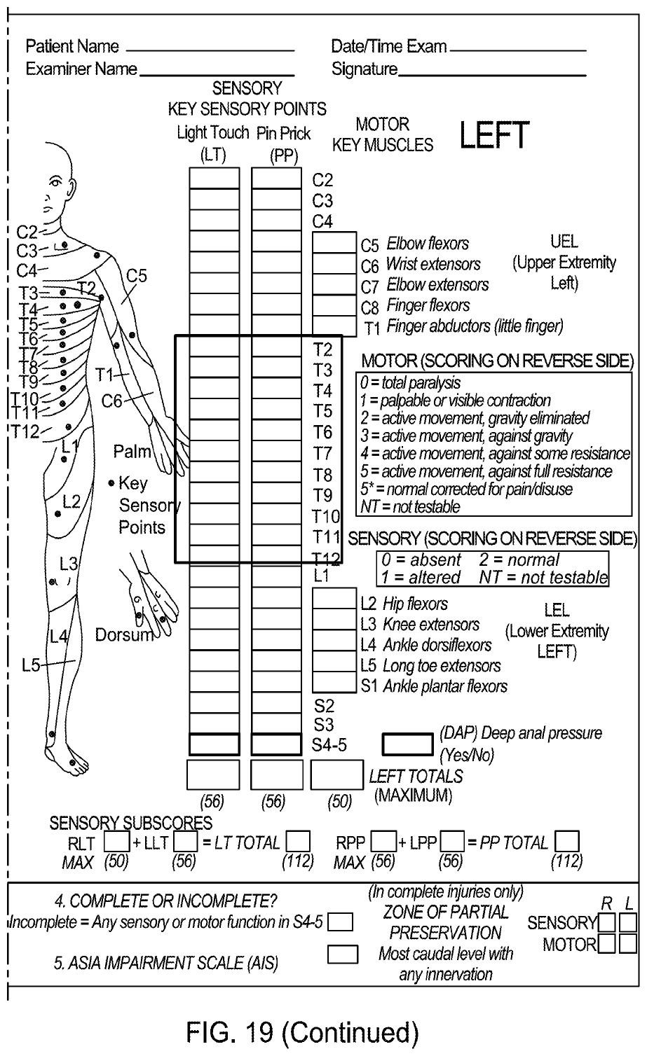

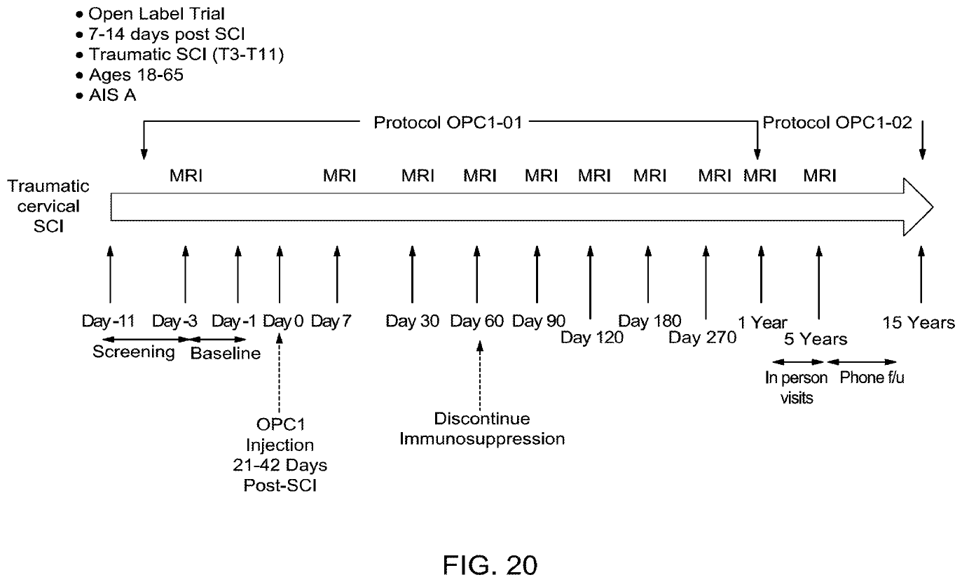

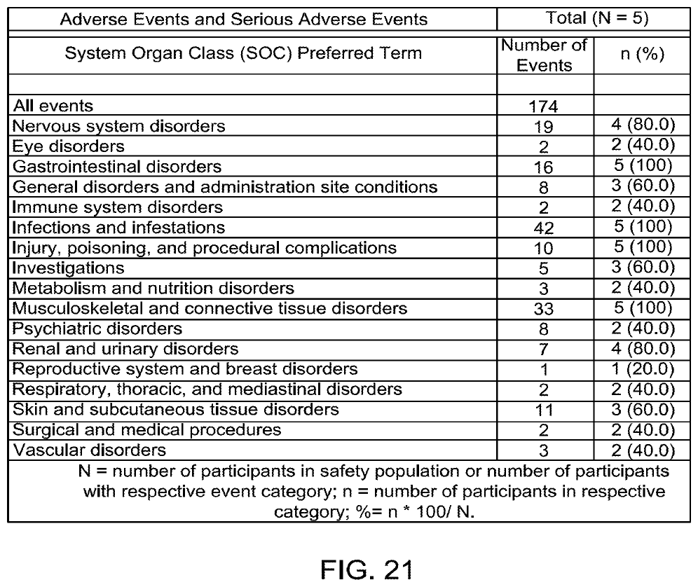

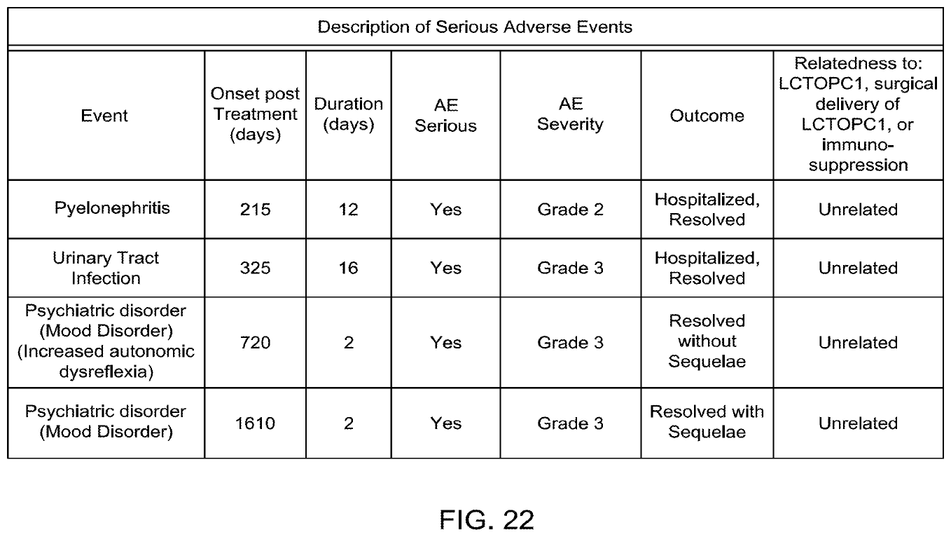

The development of the improved manufacturing process focused on a more controlled directed differentiation of H1 towards OPC, guided by a specific sequence of growth factors and small molecules to inhibit or direct differentiation pathways using cell therapy grade materials when possible (as detailed in FIG. 11). Moreover, the new process reduces the lengthy aggregate phase used by Geron, from 26 days directly from pluripotent cell state, which is prone to spontaneous aberrant differentiation, to 7 days, following 14 days of monolayer directed differentiation of H1 cells into neuroectoderm, reducing the possibility for spontaneous differentiation in the aggregates phase. The GPOR Vs. LCTOPC1 differentiation processes are summarized in FIG. 10. The biological rationale for the signaling sequence of inducing and inhibitory factors of the improved differentiation process is described in FIG. 11. Additionally, new in-process controls (IPCs) were added to better monitor and characterize the differentiation process, as detailed in FIG. 12.

Materials used to manufacture of OPC1 cells (both the original GPOR and the modified processes) are summarized in Table 17.

TABLE 17
Materials used during the production of OPC cells (GPOR and
LCTOPC1 processes).

| | Parameter | GPOR Process | LCTOPC1 Process |
|---|---|---|---|
| Stage I-hESCs | Culturing | XVIVO 10 | mTeSR Medium |
| H1 expansion | System | Medium | ReleSR |
| | | Collagenase/ | rhLaminin |
| | | mechanical | |
| | | scraping | |
| | | Matrigel | |
| Stage II- | Differentiation | GPM (DMEM/F12, T3, B-27) | |
| hESCs H1 | Medium | | |
| differentiation | Coating Reagent | Matrigel | rhLaminin |
| into OPC | Growth and | rhEGF | Dorsomorphin |
| | Differentiation | rhbFGF | PD0325901 |
| | Factors | Retinoic Acid | Retinoic Acid (RA) |
| | | (RA) | Ascorbic Acid (AA) |
| | | | rhEGF |
| | | | heat stable rhbFGF |
| | | | ROCK Inhibitor |
| | | | PDGF-A |

### Overview of the LCTOPC1 Manufacturing Process

### Stage I—H1 Expansion

Pluripotent H1 cells are thawed and cultured for 12-15 days on laminin-coated vessels in mTeSR Plus Medium. The cells are passaged and expanded using a non-enzymatic reagent ReLeSR (as described for the MCB and WCB hESC culturing).

During the expansion, the cells are morphologically assessed, and at the end of 3 passages (before the initiation of differentiation process), hESC pluripotency is evaluated by flow cytometry-based

Stage II—H1 Differentiation into OPC1

From day 0 of differentiation until the end of the process, the cells are cultured in Glial Progenitor Medium (GPM)—which is DMEM/F-12 supplemented with B27 and T3.

Day 0-7—on day 0, when the H1 culture reaches the required criteria which is defined by lactate concentration and cell morphology, the differentiation process is initiated by changing the medium for the expanded pluripotent hESC cultured on laminin-coated vessels as follows. On days 0-3, GPM medium is supplemented with Retinoic Acid (RA), Dorsomorphin and PD0325901, in order to direct the differentiation process towards the neuroectoderm pathway (Kudoh, Wilson et al. 2002). Dorsomorphin inhibits Bone Morphogenic Protein (BMP) signaling (SMAD pathway) and therefore inhibits mesoderm and trophoblast differentiation (Li and Parast 2014). PD0325901 inhibits downstream bFGF signaling at MEK1 and MEK2, and inhibits pluripotency and endoderm differentiation (Sui, Bouwens et al. 2013). In summary, inhibition of pluripotency, endoderm, mesoderm and trophoblast formation in addition to activation of the RA signaling pathway, promotes neural tube (neuroectoderm) formation (Watabe and Miyazono 2009, Sui, Bouwens et al. 2013, Li and Parast 2014, Patthey and Gunhaga 2014, Janesick, Wu et al. 2015). On days 4-7, the culture is supplemented with Retinoic Acid and Ascorbic Acid to continue neuroectoderm differentiation induction (Duester 2008).

Day 7-14—On day 7 the cells are enzymatically harvested using TrypLE Select, and then seeded as a monolayer culture from day 7 to day 14 on laminin-coated vessels and cultured in GPM supplemented with rhEGF, hsFGF and ROCK inhibitor (ROCK Inhibitor only for the first 48 hours) (Hu, Du et al. 2009, Patthey and Gunhaga 2014, Zheng, Li at al. 2018).

Day 14-21—On Day 14, in order to promote neurobody (NB) aggregate formation, the cells are enzymatically harvested using TrypLE Select, and cultured for a week as a dynamic suspension culture in GPM supplemented with ROCK inhibitor (for the first 48 hours), and rhEGF and hs-rhFGF throughout.

Day 21-42—On Day 21 the aggregates are plated back as an adherent culture on laminin-coated vessels in GPM supplemented with rhEGF and PDGF (Ota and Ito 2006, Koch, Lehal et al. 2013), and then on Day 28, the cells are harvested enzymatically using TrypLE Select, and expanded as an adherent culture on laminin-coated vessels in GPM supplemented with rhEGF and PDGF until days 35-42 for final expansion and maturation, with enzymatic passaging every ˜7 days using TrypLE Select.

At the end of the expansion process, the OPC1 cells are harvested using TrypLE Select and cryopreserved in CryoStor® 10 (CS10; BioLife Solutions, Inc.) cryopreservation solution as a Thaw-and-Inject (TAI) formulation. The LCTOPC1 production process flow is depicted in FIG. 12.

In-Process Control tests are performed at every key step during the differentiation process of hESC to OPC1, as depicted in FIG. 12. Biomarker proteins and mRNA expression are assessed using multicolor Flow Cytometry (FCM) and qPCR methods (respectively). The cells are tested for the expression of OPC1, epithelial, mesodermal, astrocytes and neuronal biomarkers, and residual hESC. In addition, viability, cell yield and metabolic activity (e.g., lactate) are assessed during the process. Lactate concentration is used as indicator for initiating differentiation starting on day 0, and on day 21 as a surrogate to cell counting in order to determine the surface area required for aggregate plating for pre-OPC generation and expansion.

### Proposed Cmc Comparability Testing

OPC1 will be manufactured according to the improved process, released according to revised release parameters, and cryopreserved. LCTOPC1 DP will be compared to Geron's manufactured representative batches and characterized with the updated analytical methods used for the release of the OPC1 manufactured via the new process. The plan will include testing of attributes used as release criteria for GPOR plus additional markers that were identified. The suggestion for comparison is based on quality attributes that characterize the Drug Product as described in Table 18.

The side-by-side comparison between LCTOPC1 and GPOR OPC1 batches will be based on statistical analysis, calculating the expected range for quantitative measurements of the quality attributes from GPOR OPC1 batches. The values of those quality attributes measured in LCTOPC1 batches will be assessed in relation to those expected ranges for the quality attributes tested. The comparability data analysis is expected to establish reproducible release criteria for the LCTOPC1 process and demonstrate that LCTOPC1 has low batch to batch variability.

The tested quality attributes will include viability, identity/purity, impurity/non-targeted population, gene profiling, and function/potency assays for 2-3 representative GPOR and LCT OPC1 batches each.

Suggested comparability quality attributes are as follows: Viability—a critical quality attribute of any live cell drug product; Identity/Purity-assessment of characteristic oligodendrocyte progenitor cell protein markers: NG2, GD3, PDGFRα and PDGRFβ; Non-targeted cells population/impurities-(i) Residual H1 hESC from starting material-human embryonic stem cells protein markers TRA-1-60 and Oct-4 as a potential source for teratogenic agents combined in a multi-color Flow Cytometry test. TRA-1-60 and Oct-4 are commonly known and used markers for embryonic stem cells and were used previously as an OPC1 release criteria, and (ii) Assessment of potential aberrant differentiation paths (Epithelial cells protein markers: Keratin 7, Claudin-6, EpCAM and CD49f known epithelial markers, Mesodermal cells protein markers CXCR4 and CD56 as known mesodermal and cartilage markers, Astrocytes cells protein marker GFAP as known astrocytes marker, Neuronal phenotype cells protein marker b-Tubulin 3 as known neuronal marker, Mesenchymal cells mRNA OLRI that induces epithelial-mesenchymal transition, Endoderm cells mRNA markers of FOXA2, SOX17 and AFP as known endodermal markers, and does not include previously used Nestin and α-actinin attributes, since new data indicate that Nestin is not specific for OPC, but rather a marker for NSC and other cell types and α-actinin can be effectively replaced by combination of CXCR4/CD56. CXCR4 is expressed in definitive endoderm and mesoderm); In-vitro Cells Function/Potency-(i) Decorin secretion—a small secreted cellular matrix proteoglycan, as a potency indicator for OPC1. Decorin expressed by neurons and astrocytes in the central nervous system attenuates scar tissue formation, inhibits cavitation and promotes wound healing. The detailed rationale for the decorin secretion as a proposed potency assay is discussed in Example 10. (ii) OPC1 cells migration in response to platelet-derived growth factor-BB (PDGF-BB) which is important for cell motility at the injury site to ensure broadest anatomical coverage from a single injection location in the spine. (iii) Development of new potency test for assessment of the maturation and myelinization potential of OPC1. This assay is based on an essential function of OPC1 cells-remyelination of denuded axons. In this assay, OPC1 cells are thawed and plated in a specific media in a 3D tissue culture environment (e.g., Matrigel or Nanofiber tubes) that should induce the maturation of OPC1 into Oligodendrocytes (OL).

The 3D environment nanotube mimics denuded axons in order to induce myelinization activity by OPC1 cells in a simple in-vitro setting. The assay will measure secretion of proteins associated with OL function (MBP and decorin) and will test OL protein markers (MBP, 04, MAG, MOG) expression by immunocytochemistry. The assay is currently being developed as a Proof-of-Concept (POC) and will be established if it proves to be robust enough.

The proposed test panel to be used to examine GPOR OPC1 and LCTOPC1 along with proposed release criteria for LCTOPC1 can be seen below in Table 18.

TABLE 18
The proposed test panel and rationale used for process development,
release and comparability.

| | | Current | | | |
|---|---|---|---|---|---|
| | | Marker | | | Current Proposed |
| | | (GPOR | Proposed | Justification | Criteria |
| Test | Method | OPC1) | Marker/s | and Reference | (based on LCTOPC1) |
| Viability, | NC-200 | % of viable | % of viable | Critical attribute | ≥90% |
| Strength | | population | population | to assess the | |
| | | | | quality of the | |
| | | | | cell product | |

Biosafety

| | | | | | |
|---|---|---|---|---|---|
| Sterility | <USP71> | NA | NA | NA | No growth |
| Mycoplasma | <USP63> | NA | NA | NA | No growth |
| Endotoxin | <USP85> | NA | NA | NA | <2 EU/ml |

Identity

| | | | | | |
|---|---|---|---|---|---|
| OPC | Flow | % NG2+ | % GD3+ | Known Pre-OPC | ≥80% |
| | Cytometry | % Nestin+ | | marker | |
| | | | % NG2+ | Known OPC | ≥80% |
| | | | | marker | |
| | | | % PDGFRa+ | Known OPC | ≥90% |
| | | | | marker | |
| | | | % PDGFRβ+ | Essential for | ≥90% |
| | | | | maturation of | |
| | | | | oligodendrocytes | |

Non-targeted cell populations

| | | | | | |
|---|---|---|---|---|---|
| Epithelial | Flow | % Keratin 7+ | % Keratin 7+ % | Known epithelial | ≤4% |
| | Cytometry | | of Claudin-6+ | markers | ≤2% |
| | | | % of EpCAM+ | | ≤2% |
| | | | % CD49f | | ≤4% |
| hESCs | | % TRA-1- | % TRA-1- | Teratogenic | ≤0.1% |
| residual | | 60+/Oct4+ | 60+/Oct4+ | potential | |
| Mesoderm | | α-Actinin+ | % | Known | ≤1% |
| | | | CXCR4+/CD56+ | mesodermal and | |
| | | | | cartilage marker | |
| Astrocytes | | GFAP | % GFAP | Known | ≤1% |
| | | | | astrocytes | |
| | | | | marker | |
| Test | | Current | Proposed | Justification | Current Proposed |
| | | Marker | Marker/s | and Reference | Criteria |
| | | (GPOR | | | (based on LCTOPC1) |
| Neuronal | | b-Tubulin 3+ | % b-Tubulin 3+ | Known | ≤4% |
| Phenotype | | | | neuronal | |
| | | | | marker | |
| Mesenchymal | | OLR1 | OLR1 | OLR1 induces | Comparable/lower than |
| | | | | epithelial- | GPOR OPC-1 |
| | | | | mesenchymal | |
| Endoderm | | AFP | FOXA2, SOX17, | Known | Comparable/lower than |
| | | | AFP | endodermal | GPOR OPC-1 |
| | | | | markers | |

Function/Potency

| | | | | | |
|---|---|---|---|---|---|
| Decorin | ELISA | Decorin | Decorin (ng/ml) | Attenuates scar | ? 25 ng/ml |
| Secretion | | (ng/ml) | | tissue, blocks | |
| | | | | cavitation, | |
| | | | | promotes wound | |
| | | | | healing | |
| Cell | NC-200 | NA | % Migrated | Potential mode | ? 30% (+PDGFI3I3) |
| Migration | | | (PDGF-induced) | of action; | |
| | | | | promote healing | |
| | | | | in tissue | |

Preliminary comparability of R&D LCTOPC1 batches, from representative runs are presented below in Tables 19-23.

TABLE 19
Comparability data from representative GPOR OPC1 and
LCTOPC1 R&D Runs-OPC1 Identity profile by flow cytometry.

| | | | | | | LCTOPC1 | | |
|---|---|---|---|---|---|---|---|---|
| Current | Proposed | | GPOR OPC1 | | | RD | RD | RD |
| Marker | Marker | Justification | M08¹ | M22¹ | M25¹ | 11A2 | 11C | 12D1 |
| % NG2 | % GD3 | Known Pre-OPC marker | 85.4 | 89.9 | 86.4 | 90 | 87.1 | 88 |
| % Nestin | % NG2 | Known OPC marker | 47.8 | 27.8 | 18.2 | 88 | 90.5 | 95.3 |
| | % NG2 | | 91.6 | 90.7 | 91.2 | 93.9 | 91.3 | 92.7 |
| | (*MG) | | | | | | | |
| | % PDGFR_{α} | Known OPC marker | 42.3 | 52.2 | 55.8 | 98.9 | 98.4 | 99.2 |
| | % PDGFR_{α} | | 79.7 | 79.9 | 87 | 99 | 99 | 99.3 |
| | (*MG) | | | | | | | |
| | % NG2/ | Known OPC markers | 27 | 16.2 | 11.8 | 86.5 | 89.1 | 94.9 |
| | PDGFR | | | | | | | |
| | % NG2/ | | 78.5 | 78 | 83.8 | 93.4 | 91.1 | 92.5 |
| | PDGFRα | | | | | | | |
| | % PDGFRβ | Essential for maturation | 92.7 | 87.1 | 91.6 | 99.1 | 98.7 | 99.1 |
| | | of oligodendrocytes | | | | | | |

*MG- Cells are seeded on Matrigel overnight before FCM analysis. GPOR OPC1 formulation and freezing reduces NG-2 and PDGFR-α expression.
¹Clinical batch

TABLE 20
Comparability data from representative GPOR and
LCTOPC1 R&D Runs-non-targeted/impurities cell population profile by flow cytometry.

| | | | | | | | LCTOPC1 | | |
|---|---|---|---|---|---|---|---|---|---|
| Cell | Current | Proposed | | GPOR OPC1 | | | RD | RD | RD |
| population | Marker | Marker | Justification | M08¹ | M22¹ | M25¹ | 11A2 | 11C | 12D1 |
| Epithelial | % Keratin7 | % Keratine | Known epitherlial | 1.04 | 1.47 | 1.88 | 0.25 | 0.38 | 0.36 |
| | | % Claudin- | markers | 4.45 | 5.83 | 3.09 | 0.33 | 0.42 | 0.17 |
| | | % EpCAM | | 1.17 | 2.21 | 1.37 | 0.62 | 0.72 | 0.99 |
| | | % CD49f | | 24.3 | 21.2 | 23.6 | 0.83 | 1.26 | 2.66 |
| Astrocytes | % GFAP | % GFAP | Known astrocytes | 1.78 | 3.44 | 1.62 | 0.028 | 0.04 | 1.35 |
| Neuronal | % b-Tubulin | % β-Tubulin | Known Neuronal | 13.7 | 17.9 | 14.3 | 0.51 | 0.29 | 0.87 |
| Phenotype | III | III | marker | | | | | | |

¹Clinical batch

TABLE 21
Comparability data from representative GPOR OPC1 and
LCTOPC1 R&D Runs-hESC residuals by flow cytometry.

| | | | | | | | LCTOPC1 | | |
|---|---|---|---|---|---|---|---|---|---|
| Cell | Current | Proposed | | GPOR OPC1 | | | RD | RD | RD |
| population | Marker | Marker | Justification | M08¹ | M22¹ | M25¹ | 11A2 | 11C | 12D1 |
| hESCs | % TRA-1- | % TRA-1- | Teratogenic | 0.01 | 0.00 | 0.00 | 0.0 | 0.00 | 0.00 |
| hESCs | % TRA-1- | % TRA-1- | Teratogenic | 0.41 | 0.49 | 0.44 | 0.0 | 0.04 | 0.01 |

¹Clinical batch

TABLE 22
Comparability data from representative GPOR OPC1
and LCTOPC1 R&D Runs-non-targeted/impurities cell
population gene profile by qPCR (relative to GPOR OPC1 M08).

| | | | | | | | LCTOPC1 | | |
|---|---|---|---|---|---|---|---|---|---|
| Cell | Current | Proposed | | GPOR OPC1 | | | RD | RD | RD |
| populatio [symbol] | Marker | Marker | Justificatio [symbol] | M08 [symbol] | M22 [symbol] | M25 [symbol] | [symbol] | [symbol] | [symbol] |
| Endoderm | AFP | AFP | Known | 1.00 | 1.35 | 1.11 | N [symbol] | N [symbol] | ND |
| | | FOXA2 | endoderm | ND | ND | ND | N [symbol] | N [symbol] | ND |
| | | SOX17 | genes | ND | ND | ND | ND | ND | ND |
| Mesenchymsl | OLR1 | OLR1 | OLR1 | 1.00 | 1.31 | 1.77 | ND | ND | ND |
| | | | induces | | | | | | |
| | | | epithelial- | | | | | | |
| | | | mesenchymal | | | | | | |
| | | | transition | | | | | | |

*ND-Not Detected
¹Clinical batch [symbol]
indicates data missing or illegible when filed

TABLE 23
Comparability data from representative GPOR OPC1 and LCT OPC1
R&D Runs-in vitro function as determined by decorin secretion and migration assays.

| | | | | | LCTOPC1 | | |
|---|---|---|---|---|---|---|---|
| | | GPOR OPC1 | | | RD | RD | RD |
| Test | Justification | M08¹ | M22¹ | M25¹ | [symbol] | [symbol] | [symbol] |
| Decorin | Attenuates scar tissue, | 25.6 | 27.61 | 23.75 | 36.67 | 29.12 | 29.78 |
| Secretion (ng/ml) | blocks cavitation, | | | | | | |
| Cells Migration | Potential mode of action; | 36 | 47 | 33 | 49 | 44 | 35 |
| (% migrated, | promote healing in tissue | | | | | | |
| PDGF-bb induced) | | | | | | | |

¹Clinical batch [symbol]
indicates data missing or illegible when filed

### Review of Ectopic Tissue Formation Assessment as a Release Criterion for OPC1 Drug Product

This analysis revealed certain correlations between the available data from GLP tox studies of Geron GMP batches to the expression level of purity and impurities/non targeted cell population specific markers.

The retesting results and analysis show correlation between impurities and batch failure in vivo, indicating that LCTOPC1, characterized by significant lower levels of impurities and high purity profile, has a greatly reduced likelihood of cyst formation in vivo than does GPOR OPC1.

### Summary

Preliminary comparability data of representative GPOR and LCTOPC1 batches demonstrate: GPOR OPC1 and R&D LCTOPC1 demonstrate similar OPC1 identity/purity profile profiles, except for the PDGFR-α biomarker which is higher in R&D LCTOPC1 and may result from the improved OPC1 manufacturing process (directed differentiation). LCTOPC1 has lower levels of impurities from epithelial, astrocytes and neuronal non-targeted cells, compared to GPOR OPC1. Both LCTOPC1 and GPOR OPC1 have very rarely detectable residual hESC (detected by % TRA-1-60+/Oct4+); however, GPOR OPC1 has a higher percentage of multipotent cells compared to LCTOPC1 as demonstrated by populations of cells exhibiting TRA-1-60+/Oct4− and CD49f+. LCTOPC1 has lower levels of endoderm and mesenchymal non-targeted cells gene expression, compared to GPOR OPC1. LCTOPC1 and GPOR OPC1 demonstrate similar decorin secretion and migration capacity.

Conclusions: the data accumulated to date show that LCTOPC1 drug product manufactured with an improved process, generated from a defined cell banking system by a highly-reproducible and tightly-controlled differentiation process and monitored with updated analytical methods, presents similar essential quality attributes to GPOR OPC1, but benefits from overall higher expression of purity markers and lower expression of an impurity/non-targeted population markers. Thus, potentially increasing the safety profile for LCTOPC1 drug product.

### References for Example 11

Duester, G. (2008). “Retinoic acid synthesis and signaling during early organogenesis.” Cell 134 (6): 921-931.
Hu, B. Y., Z. W. Du, X. J. Li, M. Ayala and S. C. Zhang (2009). “Human oligodendrocytes from embryonic stem cells: conserved SHH signaling networks and divergent FGF effects.” Development 136 (9): 1443-1452.
Janesick, A., S. C. Wu and B. Blumberg (2015). “Retinoic acid signaling and neuronal differentiation.” Cell Mol Life Sci 72 (8): 1559-1576.
Koch, U., R. Lehal and F. Radtke (2013). “Stem cells living with a Notch.” Development 140 (4): 689-704.
Kudoh, T., S. W. Wilson and I. B. Dawid (2002). “Distinct roles for Fgf, Wnt and retinoic acid in posteriorizing the neural ectoderm.” Development 129 (18): 4335-4346.
Li, Y. and M. M. Parast (2014). “BMP4 regulation of human trophoblast development.” Int J Dev Biol 58 (2-4): 239-246.
Ota, M. and K. Ito (2006). “BMP and FGF-2 regulate neurogenin-2 expression and the differentiation of sensory neurons and glia.” Dev Dyn 235 (3): 646-655.
Patthey, C. and L. Gunhaga (2014). “Signaling pathways regulating ectodermal cell fate choices.” Exp Cell Res 321 (1): 11-16.
Sui, L., L. Bouwens and J. K. Mfopou (2013). “Signaling pathways during maintenance and definitive endoderm differentiation of embryonic stem cells.” Int J Dev Biol 57 (1): 1-12. Watabe, T. and K. Miyazono (2009). “Roles of TGF-beta family signaling in stem cell renewal and differentiation.” Cell Res 19 (1): 103-115.
Zheng, W., Q. Li, C. Zhao, Y. Da, H. L. Zhang and Z. Chen (2018). “Differentiation of Glial Cells From hiPSCs: Potential Applications in Neurological Diseases and Cell Replacement Therapy.” Front Cell Neurosci 12:239.

### Example 12. Use of a Parenchymal Spinal Delivery (PSD) System

Described herein is a parenchymal spinal delivery system (FIGS. 39-41) which allows for methods of improving a neurological function in a subject having a spinal cord injury. In embodiments, the method includes administering to the subject one or more doses of an oligodendrocyte progenitor cell population using the PSD system shown in FIGS. 39-41.

In other embodiments, the oligodendrocyte progenitor cell population includes an increased proportion of cells positive for oligodendrocyte progenitor cell marker NRG2 (neuregulin 2) and a reduced expression of non-OPC markers including CD49f, CLDN6 and/or EpCAM.

The PSD system includes a needle/flow path assembly, a platform, an XYZ manipulator, syringe, and microinjection pump and foot pedal (see FIG. 39).

In certain embodiments, the PSD system described herein offers particular advantages. For example, the system offers stability and control by eliminating motion between the platform, the XYZ manipulator and the injection needle. Moreover, the system (or device) requires no cessation of ventilation, where it attaches directly to the patient and synchs with the patient's breathing motion, and the magnetic needle provides stabilization from micromotion due to heartbeats. In other advantages, the system is easy to use in a clinical setting, at least because it is smaller and requires fewer components, it is easily assembled prior to surgery, it requires a single hand operation for XYZ positioning, it provides for accurate needle depth insertion, there is straightforward cleaning and sterilization, and it is compatible with OPC1 TAI formulation and eliminates prior-day dose prep.

All references referred to herein are incorporated by references in their entireties, for all that is taught therein.

While the present disclosure has been described with reference to particular embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the scope of the present disclosure. Therefore, it is intended that the present disclosure not be limited to the particular embodiments disclosed as the best mode contemplated for carrying out the present disclosure, but that the present disclosure will include all aspects falling within the scope and spirit of the appended claims.

### Example 13. PSD System Magnetic Needle Performance

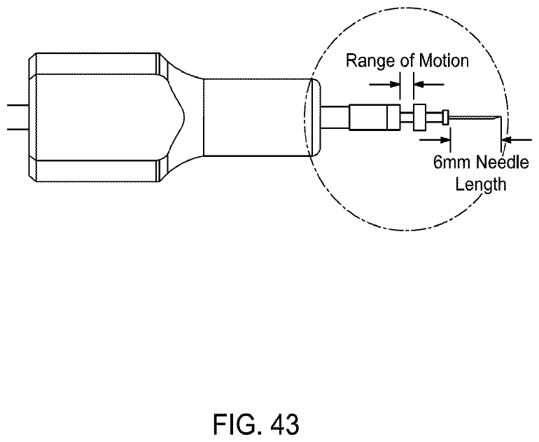

The purpose of this testing was to determine the magnetic repulsion force characteristics through the full range of motion of the magnetic needle component of the magnetic needle and flowpath assembly of the PSD system. The scope of the following description is specific to the magnetic needle component of the magnetic needle and flowpath assembly of the PSD system. The magnetic needle repulsion force was tested using a total of fifteen (15) magnetic needles. The magnetic repulsion force and displacement for each sample was measured through the full range of motion of the magnetic needle (FIG. 43).

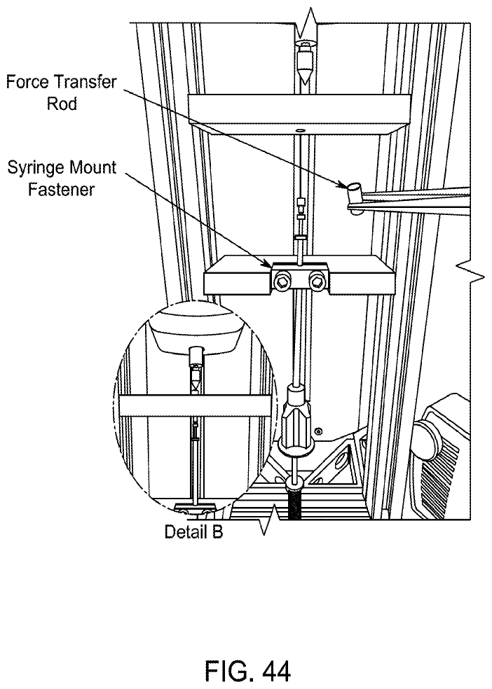

The magnetic needle assembly was secured to the fixture as shown in FIG. 44. Each sample was loaded into the test fixture such that the needle tip was pointed vertically upward and allowed to float.

Results and Discussion. The magnetic repulsion force and displacement for each sample was measured to characterize performance of the magnetic needle through its full range of motion. At its maximum range of motion, the average magnetic repulsion force of the floating needle from the 15 samples tested was determined to be 63.15 (7.74) gf at the corresponding average displacement of 6.85 (0.50) mm.

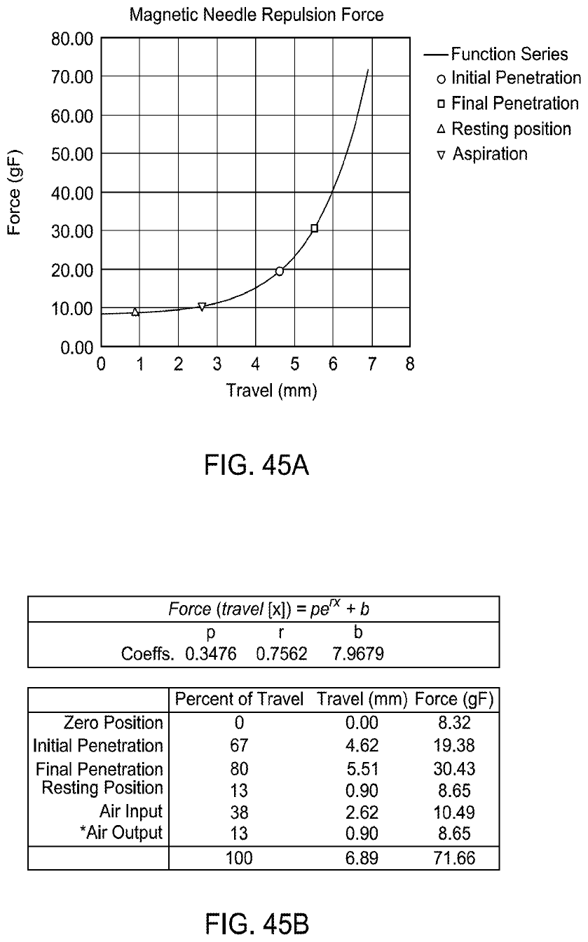

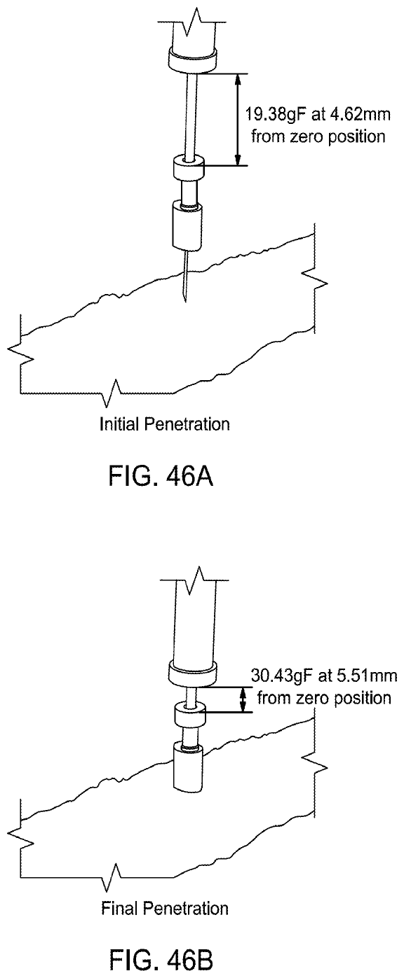

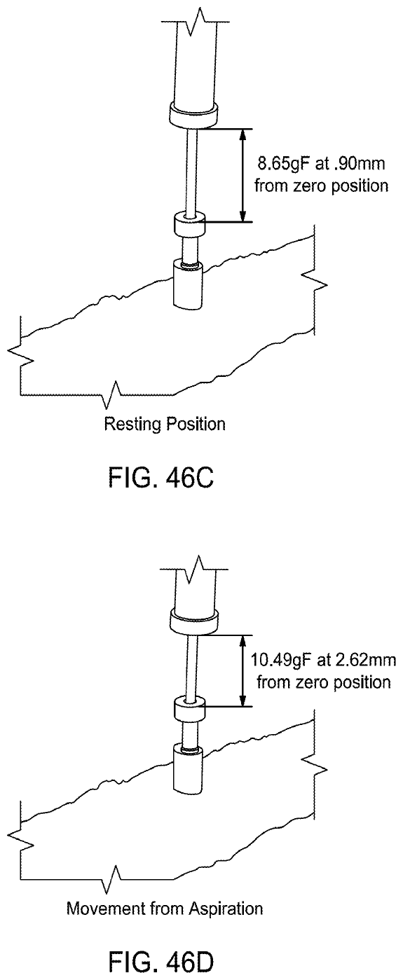

All needles performed similarly, and an average characteristic load (force) vs displacement (travel) curve was created by combining the data from all samples into a single curve. Video analysis was then performed on the simulated use of the magnetic needle in a pig spinal cord model throughout the injection process. From this analysis, magnetic force to deflection correlations were made at specific points of use: (1) at initial penetration, (2) at final penetration, (3) at resting position, and (4) during aspirations. See FIGS. 45A, 45B, and 46A-46D.

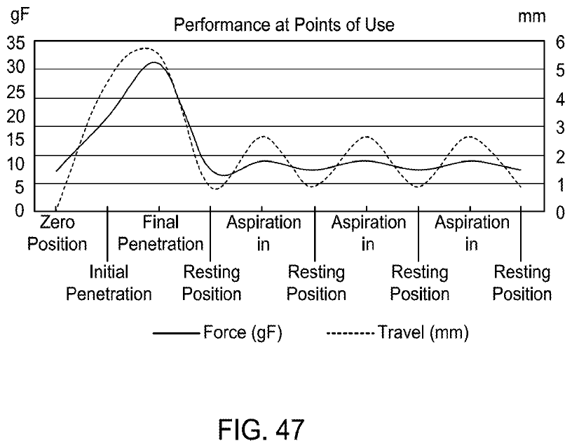

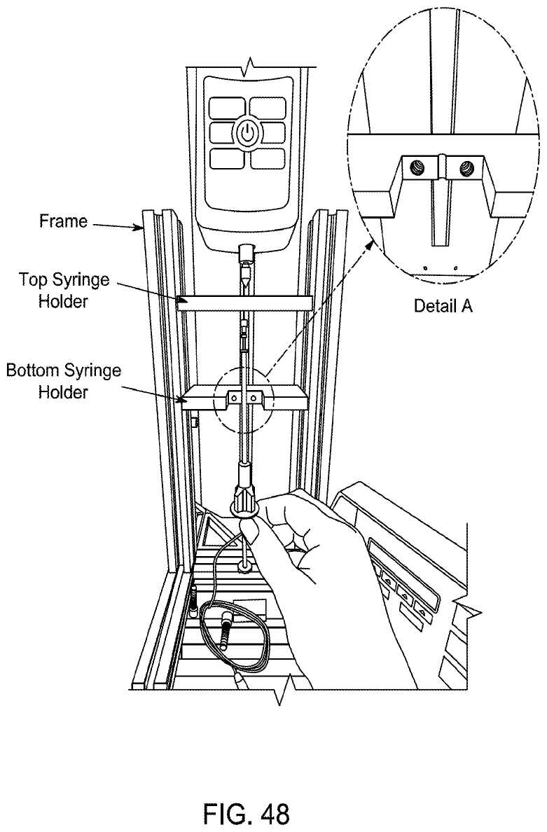

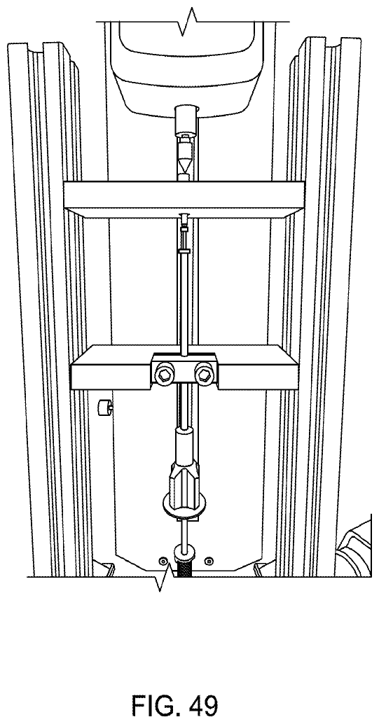

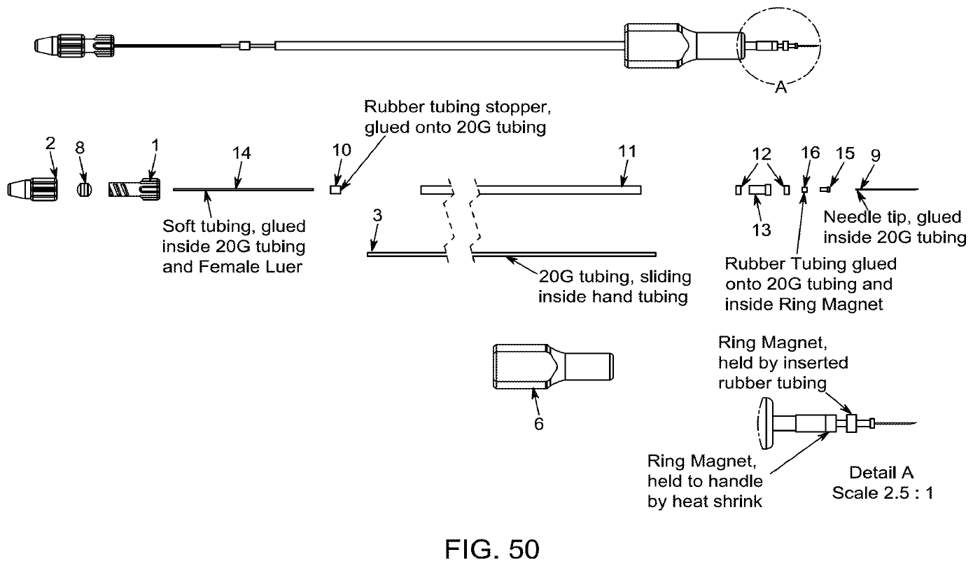

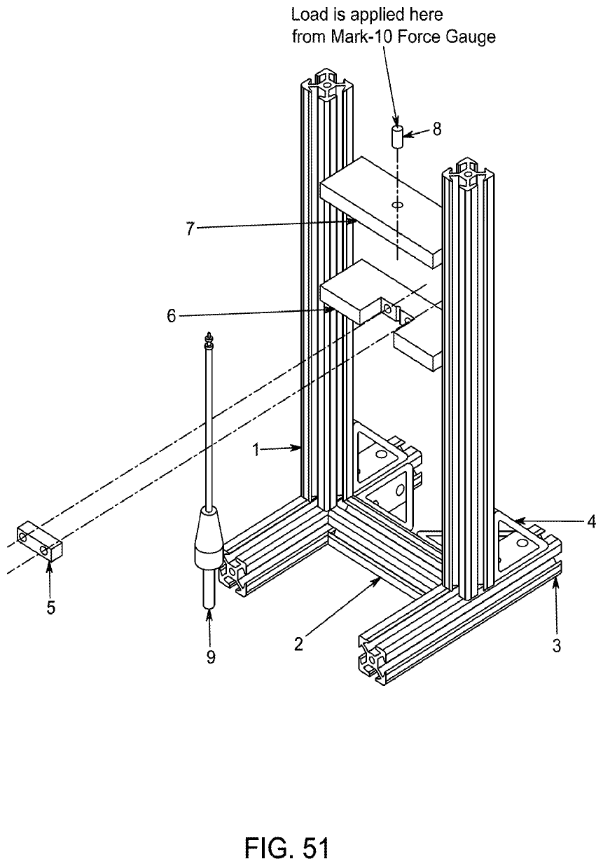

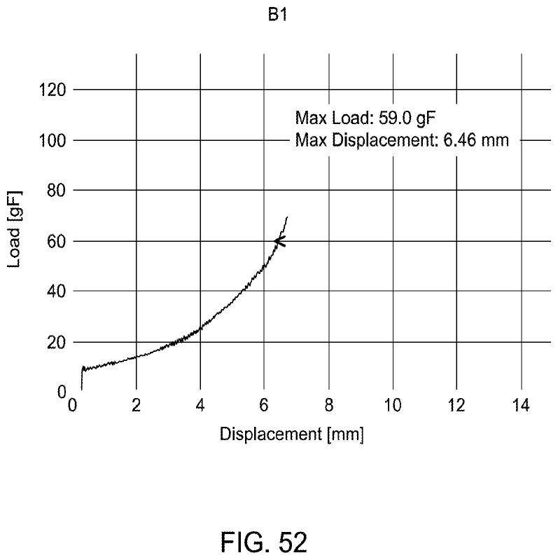

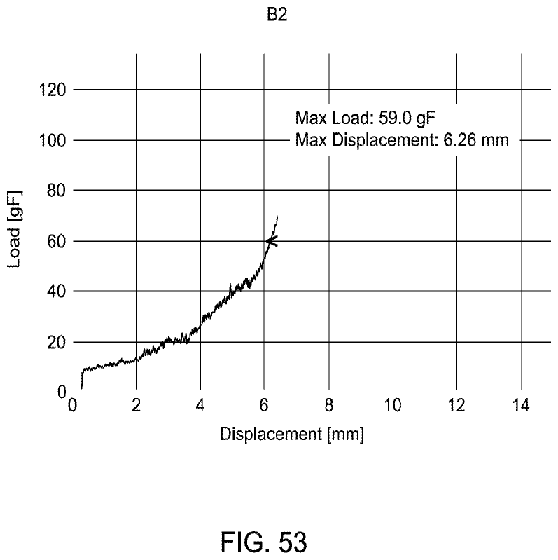

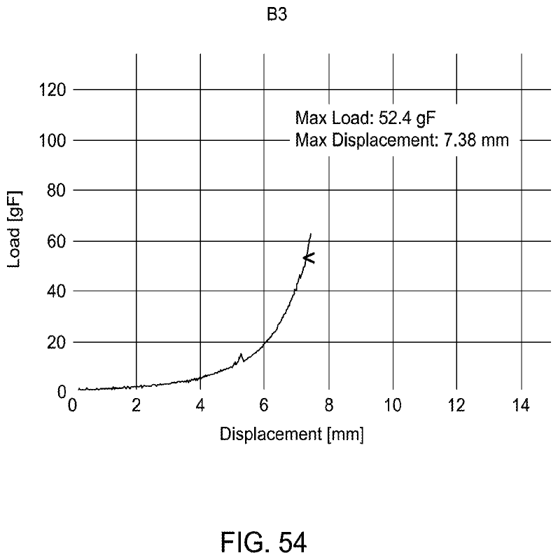

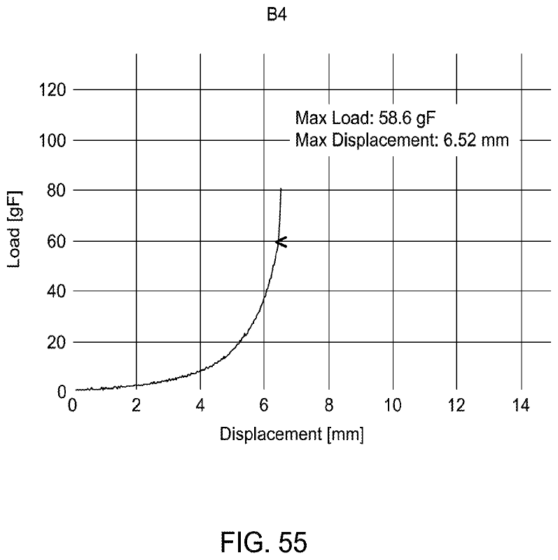

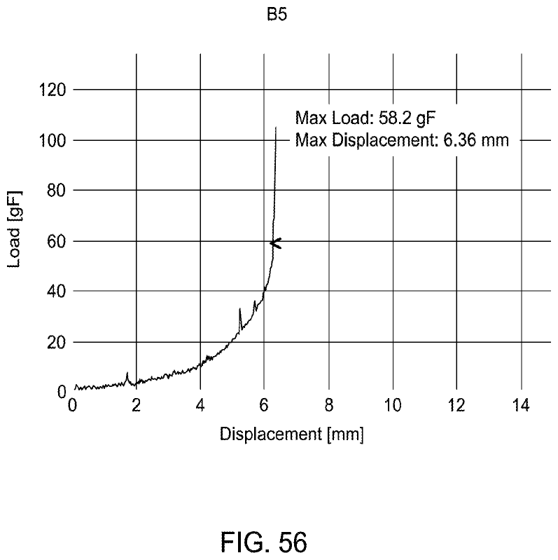

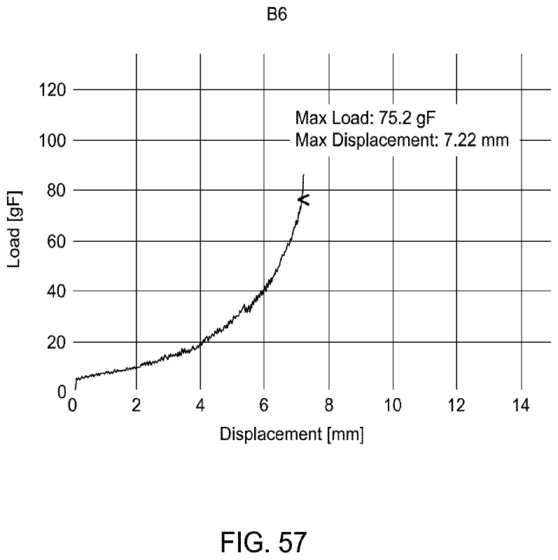

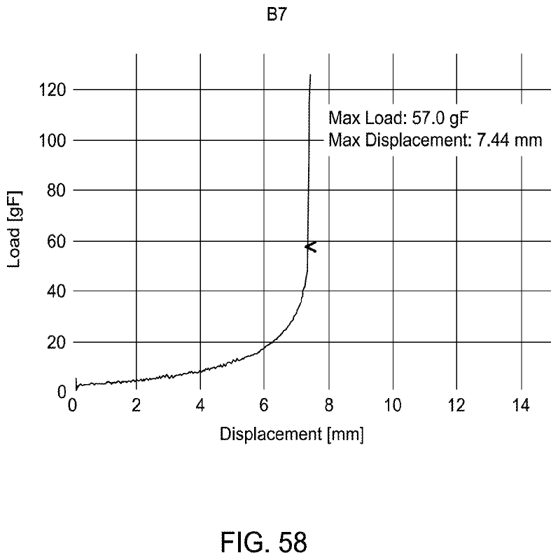

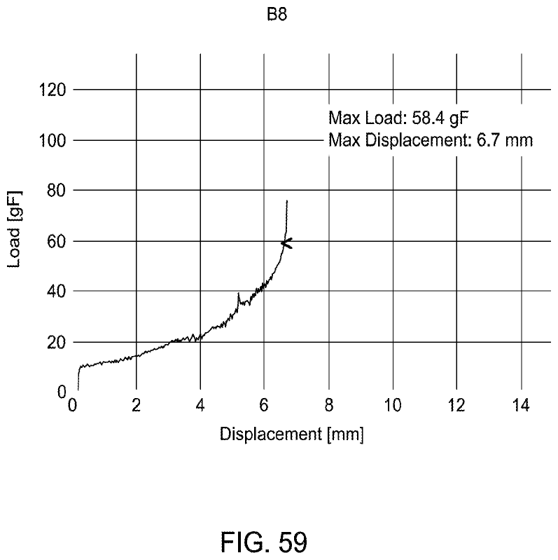

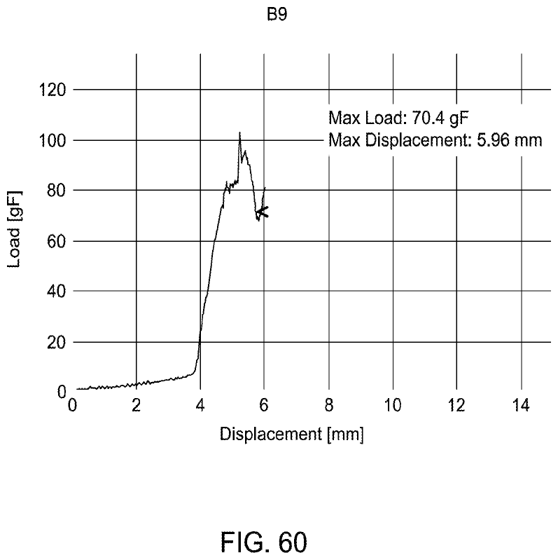

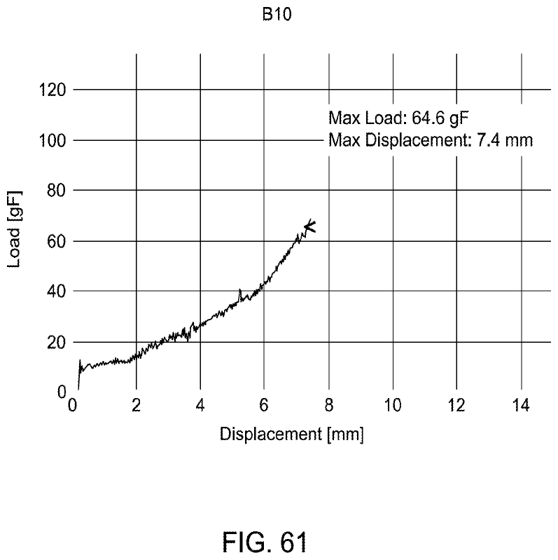

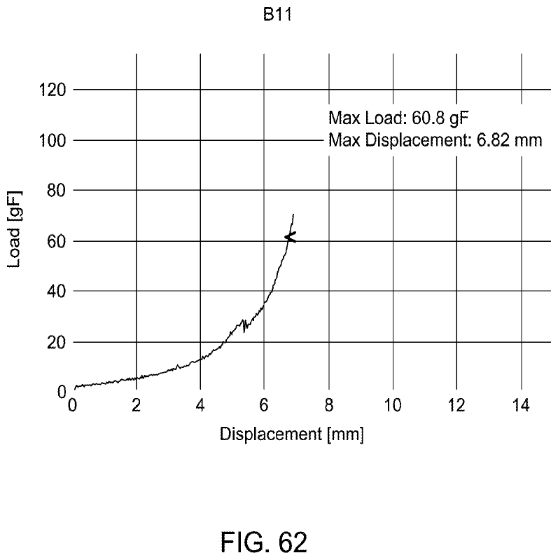

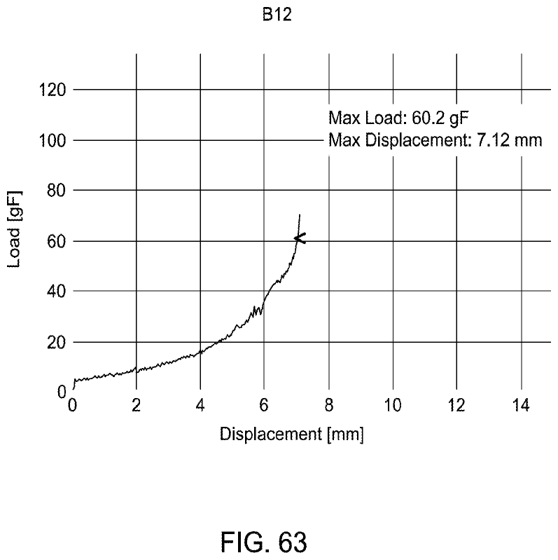

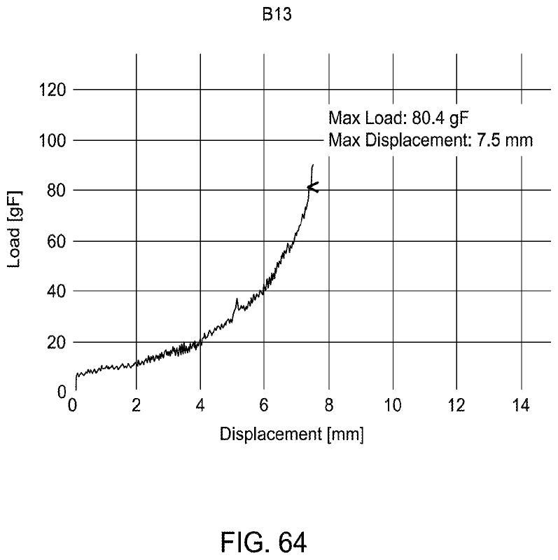

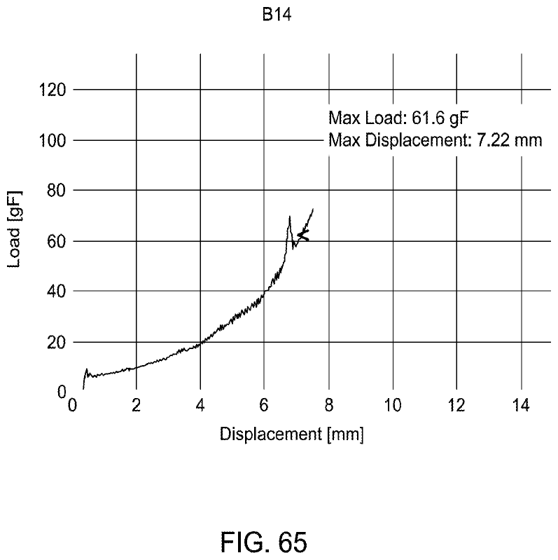

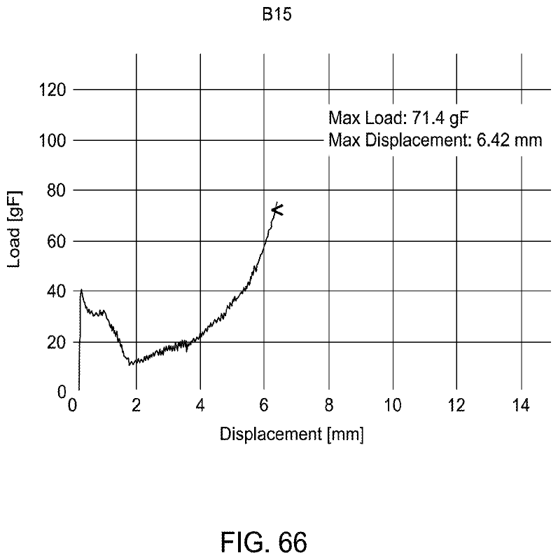

During insertion of the 6 mm needle, the penetration force was measured to be 19.38 gF at 4.62 mm of travel. The insertion force gradually increases during injection and at final penetration, the magnetic load was determined to be 30.43 gF at 5.51 mm of needle travel. After the needle is fully inserted to the stop feature, the drive pressure on the needle is reduced until no visible indication in the dura can be seen and the needle is at rest. At the resting position, the magnetic needle is at 0.90 mm of travel having a magnetic force of 8.65 gF. From this rest position, aspiration movements increase the magnetic needle travel up to 2.62 mm at a load of 10.49 gF. See FIG. 47 for illustration of the performance of the magnetic needle at these defined points of use.

The measured magnetic needle deflection during simulated respiration is approximately 1.72 mm, which is the difference between the rest position and the aspiration position. In actual human patients, spinal cord and dura mater dynamics have been quantitively analyzed and reported to range from 0.01-0.84 mm for spinal cord pulsation amplitude and range from 0.01 to 0.38 for dural pulsation amplitude. It was found that the spinal cord and dura mater may move completely independently of each other due to respiration and heartbeat (Kimura et al. Eur Spine J 21:2450-2455 (2012)). Thus, the combined movements may be as much as 1.22 mm when synchronized movement of the spinal cord and dura mater occurs. The total spinal cord movement value from literature (1.22 mm) is comparable to the video analysis measurements (1.72 mm) from the pig spine model.

Conclusion. The magnetic repulsion force of the PSD system magnetic needle was characterized throughout its full range of motion and at specific points of use. The magnetic repulsion forces of the magnetic needle at points of use were found to be reasonable and not excessive. During use, the magnetic needle does not damage tissue due to excessive force and the needle maintains proper position at each point of use. Displacement and magnetic force of the floating magnetic needle are adequate to maintain proper needle placement during respiration and heartbeat. These measured movements from video analysis are comparable to movement values reported in the literature.

### Example 14. Biodistribution of OPC1 Cells With PSD System

A quantitative polymerase chain reaction (qPCR) assay, the LCT Biodistribution Assay, was used to measure the distribution of GPOR OPC1 or LCT OPC1 cells in the tissues and cerebral spinal fluid (CSF) of Yucatan minipig at 2 days following GPOR OPC1 or LCT OPC1 cells administration.

Yucatan minipig spinal cord, brain (cerebrum, cerebellum, midbrain, medulla, pons), heart, liver, lung, kidney, spleen, and cerebrospinal fluid (240 unique specimens) collected 2 days following GPOR OPC1 or LCT OPC1 cells administration were analyzed in this study.

The animal IDs used for biodistribution testing are 7149, 7145, 7141, 7152, 7195, 7189, 7196, 6858, 7265 and 7252, as listed in Table 25 below.

TABLE 24
List of specimens received for each minipig.

| Organ/Tissue/Body Fluids | ResearchDx Tissue ID |
|---|---|
| SC-TX1 (Spinal cord injection site 1) | T1 |
| SC-TX2 (Spinal cord injection site 2) | T2 |
| SC-R1 (Spinal cord Rostral 1) | T3 |
| SC-R2 (Spinal cord Rostral 2) | T4 |
| SC-R3 (Spinal cord Rostral 3) | T5 |
| SC-C1 (Spinal cord caudal 1) | T6 |
| SC-C2 (Spinal cord caudal 2) | T7 |
| SC-C3 (Spinal cord caudal 3) | T8 |
| SC-C4 (Spinal cord caudal 4) | T9 |
| SC-C5 (Spinal cord caudal 5) | T10 |
| SC-C6 (Spinal cord caudal 6) | T11 |
| SC-C7 (Spinal cord caudal 7) | T12 |
| SC-C8 (Spinal cord caudal 8) | T13 |
| CSF (cerebrospinal fluid) | T14 |
| CTX-I (cerebrum) | T15 |
| Midb-I (midbrain; Mdb) | T16 |
| Cerebellum-I (cerebellum; Ceb) | T17 |
| Med-I (Medulla) | T18 |
| Pon-I (Pons) | T19 |
| Hrt (Heart) | T20 |
| Kid (kidney) | T21 |
| Liv (Liver) | T22 |
| Lng (Lung) | T23 |
| Spl (Spleen) | T24 |

TABLE 25
List of pigs used for the study.

| Pig ID | ResearchDx Pig ID |
|---|---|
| 7141 | P1 |
| 6858 | P2 |
| 7265 | P3 |
| 7252 | P4 |
| 7149 | P5 |
| 7145 | P6 |
| 7152 | P7 |
| 7195 | P8 |
| 7189 | P9 |
| 7196 | P10 |

The controls were as follows: the positive control was nuclease free water spiked with 10000 pg of GPOR OPC1 human gDNA.

The negative control was 100 ng of Naïve Yucatan Minipig (Pig ID 6751) spleen gDNA per well (no human gDNA). There was also a no template Control, consisting of nuclease-free water.

The LCT Biodistribution Assessment Assay is a probe-based qPCR-based assay designed to detect a 232 bp region of the human Alu repeat sequence and use it to measure the distribution of human GPOR OPC1 or LCT OPC1 cells in the tissues and cerebrospinal fluid (CSF) of Yucatan minipig following administration of GPOR OPC1 or LCT OPC1 cells. Alu sequences are a heterogeneous group of primate-specific short interspersed repetitive DNA elements with an estimated frequency of 500,000 to 1 million copies per genome. Due to species specificity, small size, and high copy number, Alu elements are ideal targets for quantitative qPCR aimed at detecting human cells among the tissues and biological fluids derived from Yucatan minipigs.

This type of assay is an oligonucleotide probe-based assay and the name of the target gene or region is Alu. The sequences of the primers and probes are listed in Table 26.

TABLE 26
Oligonucleotide sequences for the
primers and probe.

| Primer/Probe | | Tm | Amplicon |
|---|---|---|---|
| set | Sequence | (° C.) | size |
| Forward | 5′-GGCGCGGTGGCTCA-3′ | 57 | 232 bp |
| Reverse | 5′-GATCTCGGCTCACTGCA | 60 | |
| | AGC-3′ | | |
| Probe | 5′-FAM-AGGAGATCGAGAC | | |
| | CATCCTGGCTAACA-IBFQ- | | |
| | 3′ | | |

An oligonucleotide probe specific to an Alu region between forward and reverse primers was constructed containing a fluorescent reporter dye FAM on the 5′ end and a quencher dye IBFQ on the 3′ end (Table 26). While the probe is intact, the proximity of the quencher dye greatly reduces the fluorescence emitted by the reporter dye by fluorescence resonance energy transfer (FRET). If the target sequence Alu is present, the probe anneals to its specific complement region and is cleaved by the 5′ nuclease activity of Taq DNA polymerase as this primer is extended. The cleavage separates the reporter dye from the quencher dye, resulting in increased signal of the reporter dye. Additional reporter dyes are cleaved from their probes with each cycle, resulting in an increase in fluorescence intensity proportional to the amount of amplicon produced. The reaction volumes are listed in Table 27.

TABLE 27
Reaction volumes.

| Components | B. Volume (μL) per 25 μL reaction |
|---|---|
| 2x GoTaq ® Probe qPCR Master Mix | D. 12.5 |
| 10x Primer/Probe Mix | F. 2.5 |
| DNA template or NTC (water) | H. 10 |

TABLE 28
Thermal cycling conditions.

| Step | [symbol] . Temperature (° C.) | K. Time (mm:ss) | L. Cycles |
|---|---|---|---|
| Activation | N. 95 | O. 02:00 | P. 1 |
| Denaturing | R. 95 | S. 00:03 | T. 40 |
| Annealing and | V. 56 | W. 01:00 | |
| extension | | | |

[symbol]

indicates data missing or illegible when filed

DNA was extracted from tissues and body fluids. The DNA concentration was quantified using the Nanodrop.

The qPCR assay reactions were targeted to detect the Human Alu Y DNA sequence. In addition to the specimen, each plate contained the standard curve, a no template control (NTC), a negative control, and a positive control. Each of these standards and controls were run in triplicate reactions.

The acceptance criteria were as follows. The correlation coefficient (R2) of the standard curve must be ≥0.96. The qPCR reagent Control (NTC) must test below the LOD of the assay. For the positive control, the difference between the threshold cycle (Ct) values of at least two out of three replicate reactions used for quantification must be less than or equal to 1 Ct. The Negative Control must test below the LOD of the assay. For specimens within the range of quantification, the difference between the threshold cycle (Ct) values of at least two out of three replicate reactions used for quantification must be less than or equal to 1 Ct.

The results were evaluated as follows. A specimen was reported as less than the limit of detection (<LOD) if the Ct values of at least two out of three replicate reactions showed undetermined, or if the specimen's mean Ct was greater than the mean Ct of the LOD. If the quantification of a specimen exceeded the upper end of the assay's range, it was reported as greater than 10,000 pg (>10,000). Quantifications of human gDNA were reported in pg per 100 ng of minipig DNA, or pg per 40 μL body fluid.

For CSF, results are reported as the quantity (pg) of human gDNA detected in the DNA extracted from 40 μL CSF. Specimens are reported as less than the limit of detection (<LOD) if the Ct values of at least two out of three replicate reactions showed undetermined, or if the specimen's mean Ct is greater than the mean Ct of the LOD (3.2 pg). If the quantification of a specimen exceeded the upper end of the assay's range, it was reported as greater than 10,000 pg (>10,000). Three outliers were excluded from the analysis due to the difference of the replicate Ct value being greater than 1 from the other two replicates' Ct values.

Conclusion. A column-based method was used to extract gDNA from various Yucatan Minipig tissues and biofluid. A TaqMan-based qPCR assay was then used to measure the distribution of human GROP OPC1 or LCT OPC1 cells following transplantation into Yucatan Minipigs. The qPCR result determined that human gDNA was not detected in any peripheral organ gDNA (heart, kidney, liver, lung and spleen) from pigs sacrificed at 2 days after transplantation. Two days after transplantation, spinal cord tissues had the highest level of human gDNA quantities detected across tissue types. Trace amounts were detected in some other central nervous system tissues (cerebrum, cerebellum, midbrain, medulla, pons and CSF) in few animals.

### Example 15. Assessment of Intraspinal Injection Procedure Using the PSD System in the Adult Yucatan Pig: Neurological and Spinal Histopathological Study

### Introduction and Study Objectives

Rationale for Species Selection. While rodent models are appropriate for many aspects of the development of a cellular therapeutic, to fully answer questions about the potential clinical distribution of injected cells, it is important to mimic the administration route and equipment to be used in the clinical delivery as closely to humans as possible. In the specific case of an intraparenchymal spinal cord injection of cellular test article, an additional factor to model is the volume and directional flow of CSF which may “carry” the cells during the acute post-transplantation period. As the rodent CSF volume is quite small, this theoretical route of distribution is not accurately reflected in the rodent model.

Conversely, the spinal cord size and pulsation in adult pig is similar to adult human (i.e., dorso-ventral movement of the spinal cord observed after dura opening; it is primarily caused by respiration). Accordingly, the use of pig model will permit an effective testing of the Parenchymal Spinal Delivery System; in particular the magnetic needle which is designed to eliminate the effect of spinal cord pulsation during intra-parenchymal cell injection.

Experimental Procedures Overview. The purpose of the study was to monitor the acute safety, toxicity and biodistribution of the investigational drug product, LCTOPC1 (and an earlier process version of the same drug product, termed GPOR-OPC1), following cervical intraspinal cord injection in the immunosuppressed non-injured Yucatan pig using a custom microsurgical delivery device, the Parenchymal Spinal Delivery System. Additionally, the safety and feasibility of the administration procedure was assessed in the treated subjects by using periodic neurological assessment.

Beginning 24 hours prior to vehicle or test article administration, all animals were fasted. Immunosuppressant dosing was performed immediately before vehicle or test article administration and consisted of Methylprednisolone (Depomedrol; 10 mg/kg) administered as a single intra-operative dose intramuscularly, as well as Tacrolimus microspheres (0.05 mg/kg/day; 12 days releaseable formulation) intraoperatively administered subcutaneously. For animals sacrificed on day 14 of the study, a second dose of Tacrolimus microspheres were administered on the 10th post-operative day.

Following a cervical (C4-7) skin incision (3-6 inches long) on anesthetized animals, spinal C4-C5 segments were exposed through a dorsal laminectomy. A stainless-steel platform equipped with muscle retractors and firmly mounted XYZ manipulator (the Parenchymal Spinal Delivery System) was then placed over exposed vertebrae. The dura was then cut open to expose the dorsal root entry zone. The animals then received spinal intraparenchymal injections of the test article (LCTOPC1; n=8, or GPOR-OPC1; n=8) with the highest clinically intended dose, or vehicle (Cryostor CS10; n=4) unilaterally. The contra-lateral side was not injected to serve as a control. A total of two 100 μl injections containing Test Article (1×10⁷ cells within 100 μl vehicle) were delivered. After injections, the dura was closed using 6.0 Proline or a similar non-absorbable suture.

During the study, all animals were observed at least twice daily for morbidity, mortality or injury and weighed at least twice weekly. Detailed neurological examinations (modified Tarlov score) and observations were documented daily up until the scheduled termination on Day 2 or Day 14 after the injection of test article or vehicle.

Blood samples for clinical pathology evaluations and for serum analysis were collected from all animals prior to test article or vehicle injection and prior to the terminal necropsy. At study termination, necropsy examinations were performed, cerebrospinal fluid (CSF) and tissues were collectedfor subsequent assay of human DNA content. Histopathological review of additional samples from the collected tissues was conducted. Unstained tissue sections from the brain and spinal cord were used for histological confirmation of human cells by immunohistochemical (IHC) staining. The engrafted cells were examined for survival, integration, phenotype and signs of inappropriate growth or formation of ectopic structures. Immune cell infiltration in the surrounding tissue was also assessed to monitor signs of xenograft rejection.

This study was conducted in accordance with GLP guidances, Standard Operating Procedures (SOPs). This study was based on current International Conference on Harmonisation (ICH) Harmonised Tripartite Guidelines, and generally accepted procedures for the testing of pharmaceutical compounds.

### Study Design

All 20 animals in this study were considered suitable for enrollment based on veterinary health screen and were weighed prior to assignment to study. Using a simple randomization procedure, 12 male animals (weighing 20.4 to 26 kg) and 8 female animals (weighing 21 to 25.1 kg) were assigned to the control and treatment groups identified in the following Table 29.

TABLE 29
Group Assignments

| Group | Study | Number | Number of | | Termination |
|---|---|---|---|---|---|
| Number | Number | of Males | Females | Treatment | Time Point _{b} |
| 1 | CalMI2-130 | 2 | 2 | GPOR- | Day 2 |
| | (A) | | | OPC1ₐ | |
| 2 | CalMI2-130 | 2 | 2 | GPOR- | Day 14 |
| | (A) | | | OPC1ₐ | |
| 3 | CalMI2-130 | 4 | 0 | LCTOPC1ₐ | Day 2 |
| | (B) | | | | |
| 4 | CalMI2-130 | 2 | 0 | Cryostor | Day 2 |
| | (B) | | | CS10 | |
| 5 | CalMI2-130 | 2 | 2 | LCTOPC1ₐ | Day 14 |
| | (B) | | | | |
| 6 | CalMI2-130 | 0 | 2 | Cryostor | Day 14 |
| | (B) | | | CS10 | |

ₐAnimals received 2 × 10⁷ cells total
_{b} Termination time point calculated from the day of vehicle or cell administration

Animals were fasted on Day-1 for surgery on Day 0. Immunosuppressant dosing began on the day of test article or vehicle administration, with every animal having received a single dose of tacrolimus microspheres intraoperatively which provides a long-lasting releasable tacrolimus delivery for ˜12 days and also received an intraoperative single dose injection of Methylprednisolone.

Following a cervical (C4-7) skin incision, spinal C4-C5 segments were exposed through a dorsal laminectomy and the Parenchymal Spinal Delivery System was then placed over exposed vertebrae and used to administer two injections of 100 μl each containing 1×10⁷ GPOR-OPC1 (n=8), 1×10⁷ LCTOPC1 (n=8) or Cryostor CS10 (n=4) into the dorsal parenchyma of the exposed cervical spinal cord. The dura was closed using Proline or similar non-absorbable suture. Incision sites were closed in anatomical layers. During life, all animals were observed at least twice daily for morbidity, mortality or injury-induced neurological deficit and were weighed at least twice weekly. Detailed examinations and observations were documented daily until the scheduled termination.

At study termination, necropsy, clinical pathology and hematology and histopathology examinations were performed. From each animal cerebrospinal fluid and tissue samples were collected from the brain hemisphere ipsilateral to the test article injection site to include samples of brainstem, cerebellum, pons/medulla, hippocampus, forebrain, and olfactory bulbs, as well as samples from the lung, liver, spleen, heart, testes, and kidney. All samples were flash frozen and transferred for assessment of the persistence and distribution of human cells by quantitative polymerase chain reaction (qPCR). Biodistribution and histopathological analyses were conducted on additional tissue samples which were collected from each brain region (both hemispheres) listed above and from each of the listed peripheral organs. The samples were examined for engrafted cell survival, integration, phenotype and signs of inappropriate growth or formation of ectopic structures. Immune cell infiltration in the surrounding tissue was also assessed to monitor signs of xenograft rejection.

### Experimental Procedures

The immunosuppressant article tacrolimus was used as received from the Supplier and no adjustment was made for purity. The immunosuppressant article was prepared on each day of use as microspheres by mixing the 5 ml of saline (0.9% Sodium Chloride for Injection, USP) with the appropriate amount of tacrolimus microspheres and injected sodium chloride. When not in use, tacrolimus microspheres were stored at −20° C.

The immunosuppressant article Methylprednisolone (Depomedrol) was used as received from the Supplier and no adjustment was made for purity. The formulations were prepared on each day of use at the required nominal concentrations of 40, 20, 10, 4, and 2 mg/mL by mixing the appropriate amount of saline (0.9% Sodium Chloride for injection, USP) with the appropriate amount of Methylprednisolone. Prepared formulations were dispensed under a laminar flow hood using aseptic technique and sterile equipment into sterile amber glass serum bottles. Prepared formulations were stored at room temperature when not in use.

The vehicle Cryostor CS10 was dispensed on the day prior to, or on the day of use.

Animals received two injections containing approximately 1×10⁷ GPOR-OPC1 cells within each 100 μL volume. Animals received two injections containing approximately 1×10⁷ LCTOPC1 cells within each 100 μL volume.

All animals were fasted overnight prior to surgery. The animals were premediated via IM injection with acepromazine (1.0 mg/kg), atropine (0.05 mg/kg), ketoprofen (3 mg/kg), and Ceftiofur (2.2. mg/kg). An IV dose of cefazolin (25 mg/kg) was also administered. Anesthesia was induced with nitrous oxide (to effect by inhalation). Eye lubricant was administered, an endotracheal tube was inserted, and general anesthesia was maintained with isoflurane (to effectby inhalation) delivered in oxygen and maintained via a precision vaporizer and rebreathing anesthetic circuit. The hair was clipped again from the surgical site, and the shaved areas were cleansed with chlorohexidine scrub and solution. LRS was administered intravenously during surgery at approximately 180 to 240 mL/hour via a catheter and infusion pump.

Following a cervical (C4-7) skin incision (3-6 inches long) on anesthetized animals, spinal C4-C5 segments were exposed through a dorsal laminectomy. Gel foam soaked with thrombin was used to control bleeding as needed during the surgery. The animals were ventilated to maintain normal physiological levels of oxygen saturation and expired CO₂. A stainless-steel platform equipped with muscle retractors and firmly mounted XYZ manipulator (the Parenchymal Spinal Delivery System) was then placed over exposed vertebrae. The dura was then cut-open to expose the dorsal root entry zone. The animals then received spinal intraparenchymal injections of the test article (LCTOPC1; n=8, or GPOR-OPC1; n=8) with the highest clinically intended dose, or vehicle (Cryostor CS10: n=4) unilaterally. The contra-lateral side was not injected to serve as a control. A total of two 100 μl injections containing Test Article (1×10⁷ cells within 100 μl vehicle) were delivered. After injections, the dura was closed using 6.0 Proline or similar non-absorbable suture. Fascia from the surrounding incision was placed over the closed dura at the laminectomy site. Muscle layers were closed with 2-0 PDSII. The skin was closed with surgical staples. Sodium Chloride (NaCl) was used for irrigation, and bupivacaine (2 mg/kg) was infused into the incisions.

Immunosuppressant dosing was performed immediately before vehicle or test article administration and consisted of Methylprednisolone (Depomedrol; 10 mg/kg) administered as a single intra-operative dose intramuscularly, as well as Tacrolimus microspheres (0.05 mg/kg/day; 12 days-releasable formulation) intraoperatively administered subcutaneously. For animals sacrificed on day 14 of the study, a second dose of Tacrolimus microspheres were administered on the 10^{th} post-operative day.

Blood samples (approximately 5 to 7 mL) were collected from all animals ordered for study pre-test and on all treated animals prior to the terminal necropsies for serum analysis. The animals were fasted prior to blood collections as collections coincided with fasting for clinical pathology evaluations.

Necropsy examinations were performed under procedures approved by a veterinary pathologiston all animals euthanized at the scheduled necropsy. Under telazol sedation, the animals were euthanized by an intravenous dose of sodium pentobarbital via the jugular vein followed by transcardial perfusion with ice-cold saline.

CSF was collected from the cistema magna from euthanized animals prior to transcardial perfusion Approximately 1.0 to 3.0 mL was collected and flash frozen in liquid nitrogen.

The animals were examined carefully for external abnormalities including masses. The skin was reflected from a ventral midline incision and any abnormalities were identified and correlated with antemortem findings. The abdominal, thoracic, and cranial cavities were examined for abnormalities and the organs removed, examined, and where required, sample biopsies placed in neutral buffered formalin.

Tissue samples (each approximately 5 mm³) were collected from the brain hemisphere ipsilateral to the test article injection site to include samples of brainstem, cerebellum, pons/medulla, hippocampus, forebrain, and olfactory bulbs. The samples were flash-frozen to preserve DNA integrity.

The remaining tissue collected by protocol were placed in 10% neutral buffered formalin for standard histological processing and hematoxylin and eosin (H&E) staining for analysis by a board-certified veterinary pathologist. From each brain area sampled, an additional H&E slide, plus five unstained slides containing step sections (5 step sections, 50 to 100 micrometers apart) were processed and used for IHC. The ipsilateral H&E was compared to the contralateral for potential trauma caused by collection of frozen samples.

Peripheral tissue samples (approximately 5 mm³) from the lung (left lobe), liver (left lateral lobe), spleen, heart (apex), kidney (left) were collected, flash-frozen, and shipped on dry ice. An additional sample was collected from each tissue and stored in 10% neutral buffered formalin for standard histopathological processing and H&E staining for analysis by a board-certified veterinary pathologist.

Cross-sectional (coronal, transverse) slices of spinal cord, approximately 5 mm in thickness, were collected at intervals of approximately every 5 cm covering the rostrocaudal extent of the spinal cord. The samples were marked to maintain rostrocaudal orientation and flash frozen. The remaining spinal cord was marked to maintain rostrocaudal and dorsoventral orientation and was placed in 10% neutral buffered formalin for standard histological processing and H&E staining for analysis by a pathologist. Following the cross-sectional tissue trimming, the spinal cord tissues between the blocks surrounding the injection site were sectioned in the longitudinal/horizontal plane, to cover the longitudinal extent of these pieces of spinal cord tissue (each of which was approximately 4.5 to 5 cm in length). To maintain consistency between all sections of spinal cord submitted, the initial 4 mm section from the dorsal aspect of the cord was made at the time of trimming, providing a flat surface to embed on, rather than at the time of microtomy. The remaining ventral portion removed was retained in wet tissue. The rostrocaudal orientation was maintainedfor each section, by placing the cranial end of each spinal cord section within each block/slide near the labeled portion of the block/slide, respectively. The spinal cord was embedded off-center within the block to help maintain the above-specified orientation at the time of sectioning. Each spinal cord sectioned was additionally trimmed, as needed, into shorter pieces to adequately fit the tissue cassettes, but the rostrocaudal orientation was maintained among all sections trimmed. For each of the longitudinal segments, standard H&E staining and histopathological analysis was performed by a board-certified veterinary pathologist.

IHC detection of cells of human origin was achieved using an antibody specific for human nucleoli (Abcam, Reference No. ab190710). A chromogenic HRP polymer-based method was conducted on a Discovery Ventana Ultra automated staining instrument (Roche Diagnostics). Slides from a formalin-fixed, paraffin embedded human cell line (H441 cells) pellet were included as a positive control in each staining run to verify the procedure worked as intended. A H441 slide that received an isotype control antibody in place of the human nucleoli antibody was used as a negative control. Staining Procedure #659 was selected as the most suitable by the Principal Investigator.

Negative and positive control samples (human cell pellet) were evaluated for each staining run. There was no positive stain on the two negative control samples (scored as “0”. The positive control samples had strong staining (scored “5” using the scale below):
0=No staining
1=Minimal (rare positive cells)
2=Mild (some positive cells)
3=Moderate (at least half of cells positive)
4=Marked (most cells positive)
5=Severe (virtually all cells are positive)

This scale was also used to evaluate the study tissues.

Real-Time Quantitative Polymerase Chain Reaction (qPCR) Analysis

20 pigs divided between the Day 2 and Day 14 timepoints had samples collected after sacrifice for analysis. The following tissues were collected for analysis from each pig: spinal cord tissues (13 tissues), CSF (1 sample), brain (5 tissues), and peripheral organs (heart, liver, kidney, spleen, lung) (5 tissues total); grand total tissues per pig (20)=24. DNA was extracted and from each tissue, detected via qPCR and quantified using serial dilutions of hu gDNA as standards.

### Assay Description and Qualification

A quantitative polymerase chain reaction (qPCR) assay was used to measure the distribution of GPOR-OPC1 and LCTOPC1 cellular test article after administration into Yucatan minipigs. The qPCR-based assay was designed to detect a specific region of the human Alu repeat sequence. The assay was used to measure the distribution of human GPOR-OPC1 or LCTOPC1 cells in the tissues and cerebrospinal fluid (CSF) of Yucatan minipig following GPOR-OPC1 or LCTOPC1 cell administration. The assay was conducted in accordance with the U.S. Food and Drug Administration (FDA) Good Laboratory Practice (GLP) regulations, standards, and guidelines (21 CFR Part 58).
Specimen extractions (except for CSF) yielded gDNA with concentration ≥10 ng/μL (CV % 0.3 to 3.5% and 260/280 ratio above 1.2.
The NanoDrop reading for negative extraction control was below 2.5 ng/μL and/or had no distinguishable OD peak at 260 nm.
CSF was extracted volumetrically with 200 μL of starting material regardless of the DNA yield.

### Preparation of Samples

For tissues, the entire tissue piece received was homogenized and an aliquot removed for DNA isolation. For CSF, up to 200 μL was used for the extraction. A naïve tissue or PBS was included with each batch of specimens, and extracted in parallel with test samples to monitor for contamination during the extraction process (NEC-negative extraction control).

For tissues, the concentration of the DNA purified from each tissue was determined by absorbance at 260 nm (A260) and the concentration subsequently adjusted to 100 ng/μL with water. Ten microliters (1 μg DNA) were used in each qPCR. For samples with DNA concentrations less than 100 ng/μL but greater than or equal to 50 ng/μL, the DNA was adjusted to 50 ng/μL, and 20 μL (1 μg DNA) of the DNA preparation was run per reaction. Samples with DNA concentrations less than 50 ng/μL were run using 20 μL per qPCR. CSF samples were run volumetrically with DNA from the equivalent of up to 10 μL of biological fluid analyzed in each reaction. For all reactions, the mass of DNA analyzed was recorded.

Real Time qPCR Reaction

The qPCR amplification and fluorescence detection were performed using the ABI 7900 HT Real Time PCR instrument. To monitor for the presence of PCR inhibitors in each test sample DNA, a single reaction was prepared with up to 1 μg DNA from each sample and spiked with 1 pg of human gDNA. In addition to specimen DNA, each test plate contained one set of standards, a naïve Yucatan pig gDNA negative control (0 copy) and the qPCR reagent control (NTC). Each extraction control was included on at least one run with its corresponding specimens. These controls monitor the potential for non-specific amplification of animal model gDNA, contamination of the qPCR reagents, and contamination of specimen DNAduring the extraction process, respectively. All controls were run in duplicate reactions.

### Hematology Findings

For the groups terminated on Day 2, no meaningful differences among hematology parameters were observed. One animal receiving GPOR-OPC1 cells demonstrated an increase in ALT above the healthy range, whilst one animal receiving LCTOPC1 demonstrated an increase in hematocrit above the healthy range. There were animals in each of the three groups which demonstrated mild increases in mean corpuscular hemoglobin concentration that were considered procedure related. All other mean and individual values were considered within an acceptable range for biologic and procedure related variation.

For the groups terminated on Day 14, one animal that received GPOR-OPC1 and one which received LCTOPC1 had moderate reactive lymphocytes observed. A separate animal which had received GPOR-OPC1 showed an increase in eosinophil levels above the healthy range. One animal that received Cryostor CS10 demonstrated low levels of neutrophils. There were animals present in each of the three groups which returned samples containing WBC lower that the healthy range. Two animals that received GPOR-OPC1 showed increases in hematocrit levels, whilst one demonstrated a reduction in hematocrit. Moreover, animals of each of the three groups exhibited mild increases or reductions in erythrocytes and hemoglobin that were similar and were considered procedure related. No other meaningful changes among hematology parameters were observed in any group.

### Microscopic Assessments of Spinal Cord and Brain Tissues

There were no cells consistent with human cells (H&E evaluation) in the transplantation sites from Day 2 and Day 14 vehicle-administered animals (4 animals, 8 total sites).

In the 8 animals (4 at Day 2 and 4 at Day 14) and 16 total transplantation sites administered GPOR-OPC1 test article, cells consistent with human cells were identified in 7/16 sites. This included:
7/8 Day 2 sites
0/8 Day 14 sites

This 7-site total included 7 sites identified during the initial H&E assessment. It does not include the site where putative human cells were identified during the initial H&E assessment, but positive cells were not seen with IHC (Transplantation site 2, Animal 7609).

Within the individual 8 animals:
3/8 had cells identified in both Transplantation sites (3/4 Day 2 Animal)
1/8 had cells identified in one Transplantation site (1/4 Day 2 Animals)
4/8 did not have cells identified in either site (4/4 Day 14 Animals)

In addition to the presence of human cells in the Transplantation sites, the main additional microscopic findings were:
Mononuclear cell infiltration (generally perivascular in Day 2 samples; typically diffuse, higher grade, and extending in gray and white matter in Day 14 samples).
Rare polymorphonuclear cells were seen within the mononuclear cell infiltration but were considered to be a minor component and not scored separately.
Changes in spinal cord white matter (typically minimal to mild axonal swelling and dilation of the myelin space around axons).
Acute hemorrhage (typically Day 2 samples and attributed to the recent transplantation procedure).

The frequency and severity of these additional changes were generally higher in Day 14 sites compared to Day 2 sites. Overall, there was no major disruption of the spinal cord tissue adjacent to the Transplantation sites in any animal (e.g., necrosis and/or cavitation were not noted).

In virtually all of the 7 HuNu-positive sites (IHC evaluation), there was evidence of minimal cell disruption (characterized by either extracellular material consistent with positive stain or intracellular cytoplasmic staining in cells consistent with macrophages, suggesting phagocytosis of remnants of human cells). This change was usually a minor component of the overall positive staining was listed as either present or absent but not graded. Additionally, the evidence of cell disruption was distinguished from light brown pigment (cytoplasmic and extracellular) suggestive of hemosiderin.

Microscopic findings in the spinal cord sections rostral and caudal to the transplantation sites were generally minimal and similar to those in the transplantation site (e.g., perivascular mononuclear cell infiltrates).

No microscopic findings in the brain were interpreted as related to the test article administration.

In the remaining 8 animals (4 at Day 2 and 4 at Day 14) and 16 total sites administered LCTOPC1 test article, cells consistent with human cells were identified in 8/16 sites. (7/8 Day 2 sites and 1/8 Day 14 sites (Single site was Transplantation site 1 from Animal 7266).)

This 8-site total included 6 sites identified during the initial H&E assessment and two sites that were re-examined after the IHC data became available (Transplantation site 1 in 7196 and Transplantation site 1 from Animal 7266).

Within the individual 8 animals administered cells:
3/8 had cells identified in both Transplantation sites (3/4 Day 2 Animals)
2/8 had cells identified in one Transplantation site (1/4 Day 2 Animals and 1/4 Day 14 Animal).
3/8 did not have cells identified in either site (3/4 Day 14 Animals).

In addition to the presence of human cells in the Transplantation sites, the main additional microscopic findings were:
Mononuclear cell infiltration (generally perivascular in Day 2 samples; typically diffuse, higher grade, and extending in gray and white matter in Day 14 samples).
Rare polymorphonuclear cells were seen within the mononuclear cell infiltration but were considered to be a minor component and not scored separately.
Changes in spinal cord white matter (typically minimal to mild axonal swelling and dilation of the myelin space around axons).
Acute hemorrhage (typically Day 2 samples and attributed to the recent transplantation procedure).

The frequency and severity of these changes were generally higher in Day 14 sites compared to Day 2 sites.

Overall, there was no major disruption of the spinal cord tissue adjacent to the Transplantation sites in any animal (e.g., necrosis and/or cavitation were not noted). Additionally, the incidence of severity of mononuclear cells in vehicle-treated sites were generally low at both Day 2 and Day 14. No changes in the spinal cord white matter were noted in control sites (e.g., no axonal swelling or dilation of the myelin space).

Two sections that did not have human cells identified on the H&E sections did contain HuNu positive cells (Transplantation site 1 from Animal 7196 and Transplantation site 1 from Animal 7266). The H&E sections were subsequently re-examined and found to either have very low numbers of cells that were consistent either with host macrophages or human cells (Animal 7196, Day 2) or the individual positive cells were centrally located within a pale tissue focus surround by a large focus of mononuclear cells infiltration (Animal 7266, Day 14).

In virtually all of the 8 HuNu-positive sites, there was evidence of minimal cell disruption (characterized by either extracellular material consistent with positive stain or intracellular cytoplasmic staining in cells consistent with macrophages, suggesting phagocytosis of remnants of human cells). This change was usually a minor component of the overall positive staining was listed as either present or absent but not graded. Additionally, the evidence of cell disruption was distinguished from light brown pigment (cytoplasmic and extracellular) suggestive of hemosiderin.

In animals administered LCTOPC1, microscopic findings in the brain were rare and considered to be unrelated to the test article administration. Microscopic findings in the spinal cord sections rostral and caudal to the transplantation sites were generally minimal and similar to those in the transplantation site (e.g., perivascular mononuclear cell infiltrates).

In summary, animals administered either GPOR-OPC1 or LCTOPC1 cells (intraparenchymal) using the Parenchymal Spinal Delivery System for injection had increased numbers of cells and decreased other microscopic findings in the Transplantation sites at Day 2 compared to Day 14. Identification of cells on H&E sections generally correlated well with HuNu positive IHC staining. Overall, there was no major disruption of the spinal cord tissue adjacent to the Transplantation sites in any animal (e.g., necrosis and/or cavitation were not noted). No changes in the spinal cord white matter were noted in control sites (e.g., no axonal swelling or dilation of the myelin space).

### Tissue Collection and Processing

Approximately 2 mL of CSF was collected from the Cisterna Magna from animals immediately following euthanasia with Sodium Pentobarbital, prior to exsanguination and flash frozen in Liquid Nitrogen. Cross-sectional (coronal, transverse) slices of spinal cord, approximately 5 mm in thickness, were collected at intervals of approximately every 5 cm covering the rostrocaudal extent of the spinal cord. The samples were marked to maintain rostrocaudal orientation and flash frozen to preserve DNA integrity. Additional tissue samples (each approximately 5 mm³) were collected from the brain hemisphere which is ipsilateral to the test article injection site to include samples of the brainstem, cerebellum, pons/medulla, hippocampus, forebrain, and olfactory bulbs. These samples were flash-frozen to preserve DNA integrity.

### Identification of GPOR-OPC1 or LCTOPC1 in Peripheral Tissues of Swine

DNA was extracted from the tissues and body fluids submitted for analysis (8 levels of spinal cord, brainstem, cerebellum, pons/medulla, hippocampus, forebrain, olfactory bulb, heart, liver, lung, kidney, spleen, and CSF) using standard methods. Duplicate qPCR amplification reactions with fluorescence detection were performed for each sample to label nuclear human specific Alu DNA repeat sequences. Values were compared to a standard curve created using known amounts of human genomic DNA (gDNA) spiked into Yucatan minipig spleen homogenates to quantify the number of human-specific Alu DNA repeat gene sequences in the test sample. Values were reported numerically if they fell within the quantitative range of the assay (1 pg to 10,000 pg human Alu DNA/μg of Yucatan minipig tissue); values falling outside that range were reported as “less than the lower limit of detection” (LLD; <500 fg) or “non quantifiable” (NQ; between 500 fg and 1 pg).

In peripheral tissues (Table 30), the highest values measured were in the hearts of two animals that received Cryostor (animals number 2001 and number 2003), indicating sample contamination by human DNA. Animals number 2001 and number 2002 both received Cryostor and were the first animals in the study to undergo necropsy; measurable amounts of human DNA were detected in a number of the peripheral tissue samples from these two animals. In animals that received GRNOPCl, one animal (number 2005) had measurable levels of human DNA in the heart at the Day 2 time point. In addition, animals number 2010 and number 2011 that received GRNOPCl and were included in the Day 14 time point had measurable levels of human DNA in multiple peripheral tissues (liver, lung and spleen for both animals, and testis and kidney for animal number 2010), although the levels were still in the range of those reported for HBSS animals number 2001 and number 2003. Of note, all other animals that received GRNOPCl and were included in either termination time point had little to no human DNA in the peripheral tissues sampled.

TABLE 30
Biodistribution of GPOR-OPC1 or LCTOPC1 in Peripheral Tissues
(Expressed as Picogram Human DNA per Microgram Minipig gDNA)

| Time | Treatment | Animal | | | | | | |
|---|---|---|---|---|---|---|---|---|
| Point | Group | ID# | Heart | Lung | Liver | Spleen | Testis | Kidney |
| Day 2 | Cryostor | 7145 | | 6.05 | 2.97 | LLD | 33.58 | 5.87 |
| Day 2 | Cryostor | 7149 | LLD | 2.19 | LLD | 3.32 | LLD | LLD |
| Day 2 | GPOR-OPC1 | 6858 | 3.38 | LLD | LLD | LLD | LLD | LLD |
| Day 2 | GPOR-OPC1 | 7141 | NQ | LLD | LLD | LLD | LLD | LLD |
| Day 2 | GPOR-OPC1 | 7252 | LLD | LLD | LLD | LLD | LLD | LLD |
| Day 2 | GPOR-OPC1 | 7265 | LLD | LLD | LLD | LLD | LLD | LLD |
| Day 2 | LCTOPC1 | 7152 | 153.15 | LLD | LLD | LLD | LLD | LLD |
| Day 2 | LCTOPC1 | 7189 | LLD | LLD | LLD | LLD | LLD | LLD |
| Day 2 | LCTOPC1 | 7195 | LLD | 5.27 | 5.81 | 15.07 | 14.50 | 8.82 |
| Day 2 | LCTOPC1 | 7196 | LLD | 102.82 | 44.89 | 10.24 | LLD | LLD |
| Day 14 | Cryostor | 7254 | LLD | LLD | LLD | LLD | LLD | LLD |
| Day 14 | Cryostor | 7258 | LLD | LLD | LLD | LLD | LLD | LLD |
| Day 14 | GPOR-OPC1 | 7259 | | | | | | |
| Day 14 | GPOR-OPC1 | 7267 | | | | | | |
| Day 14 | GPOR-OPC1 | 7609 | | | | | | |
| Day 14 | GPOR-OPC1 | 7612 | | | | | | |
| Day 14 | LCTOPC1 | 7253 | | | | | | |
| Day 14 | LCTOPC1 | 7266 | | | | | | |
| Day 14 | LCTOPC1 | 7688 | | | | | | |
| Day 14 | LCTOPC1 | 7691 | | | | | | |

^{a} LLD = Less than the Limit of Detection (<500 fg)
^{b} NQ = Not Quantifiable (specimens testing between 500 fg and 1 pg)

### Identification of GPOR-OPC1 or LCTOPC1 in CSF, Spinal Cord and Brain Tissues of Swine

Human DNA was not detected in any of the CSF samples obtained.

The highest values detected in the brain tissues sampled were again measured in animal number 2001 and animal number 2002, both of which received vehicle and were necropsied first in the study for the Day 2 time point. Most animals that received GRNOPCl had measurable levels of human DNA in samples from the brainstem and pons/medulla, the two brain levels most proximal to the cervical spinal cord injection site. While histological examinations of similar brain samples failed to identify any human cells in the brain of any animal in the study, it is possible that some GRNOPCl entered the subdural space near the injection site (as occurred in the procedural pilot Study 1058-018, which preceded this study) and were collected during tissue harvest for the brainstem and pons/medulla samples. There was no histological evidence for intraparenchymal migration of the GRNOPCl from the injection site to these levels of the caudal brain; all identified GRNOPCl were in the cervical spinal cord area closest to the site of injection.

Notably, however, all measured levels in these tissues from animals that received GRNOPCl were lower than the amounts reported for animal number 2001, which received vehicle only.

TABLE 31
Biodistribution of GPOR-OPC1 or LCTOPC1 in Brain Tissues and CSF
(Expressed as Picogram Human DNA per Microgram Minipig gDNA)

| Time | Treatment | Animal | Olfactory | Fore- | Hippo- | Cere- | Pons/- | Brain- | |
|---|---|---|---|---|---|---|---|---|---|
| Point | Group | ID# | Bulb | brain | campus | bellum | Medulla | stem | CSF |
| Day 2 | Cryostor | 7145 | | 6.05 | 2.97 | LLD | 33.58 | 5.87 | |
| Day 2 | Cryostor | 7149 | LLD | 2.19 | LLD | 3.32 | LLD | LLD | |
| Day 2 | GPOR- | 6858 | 3.38 | LLD | LLD | LLD | LLD | LLD | |
| | OPC1 | | | | | | | | |
| Day 2 | GPOR- | 7141 | NQ | LLD | LLD | LLD | LLD | LLD | |
| | OPC1 | | | | | | | | |
| Day 2 | GPOR- | 7252 | LLD | LLD | LLD | LLD | LLD | LLD | |
| | OPC1 | | | | | | | | |
| Day 2 | GPOR- | 7265 | LLD | LLD | LLD | LLD | LLD | LLD | |
| | OPC1 | | | | | | | | |
| Day 2 | LCTOPC1 | 7152 | 153.15 | LLD | LLD | LLD | LLD | LLD | |
| Day 2 | LCTOPC1 | 7189 | LLD | LLD | LLD | LLD | LLD | LLD | |
| Day 2 | LCTOPC1 | 7195 | LLD | 5.27 | 5.81 | 15.07 | 14.50 | 8.82 | |
| Day 2 | LCTOPC1 | 7196 | LLD | 102.82 | 44.89 | 10.24 | LLD | LLD | |
| Day 14 | Cryostor | 7254 | LLD | LLD | LLD | LLD | LLD | LLD | |
| Day 14 | Cryostor | 7258 | LLD | LLD | LLD | LLD | LLD | LLD | |
| Day 14 | GPOR- | 7259 | | | | | | | |
| | OPC1 | | | | | | | | |
| Day 14 | GPOR- | 7267 | | | | | | | |
| | OPC1 | | | | | | | | |
| Day 14 | GPOR- | 7609 | | | | | | | |
| | OPC1 | | | | | | | | |
| Day 14 | GPOR- | 7612 | | | | | | | |
| | OPC1 | | | | | | | | |
| Day 14 | LCTOPC1 | 7253 | | | | | | | |
| Day 14 | LCTOPC1 | 7266 | | | | | | | |
| Day 14 | LCTOPC1 | 7688 | | | | | | | |
| Day 14 | LCTOPC1 | 7691 | | | | | | | |

In the spinal cord samples collected, all four animals that received vehicle had measurable levels of human DNA at multiple levels of the rostrocaudal extent of the spinal cord, reflecting signal not derived from GRNOPCl. In animals that did receive GRNOPCl, most of the higher levels of human DNA were measured in samples from spinal cord sections contained in the region of sections 1-3, which correspond to the cervical spinal cord and contain the injection site. More GRNOPC1 animals in the Day 2 termination time point group had measurable levels of human DNA in spinal cord tissues than were assayed from animals in the Day 14 termination time point group. While GRNOPCl is expected in spinal cord sections from the cervical spinal cord (based on histological results and identification of GRNOPCl using ISH), the source of human DNA detected in spinal cord levels distal to the injection site is unclear. It is highly unlikely that it reflects intraparenchymal migration, as GRNOPCl migration would be expected in all segments between the injection site and a distal location (based on extensive data collected in the rodent) and this was not observed. As described above, it may be possible that some GRNOPCl entered the subdural space near the injection site and were collected during tissue harvest for the distal spinal cord segments; of note, human DNA was also measured in a number of the most lumbar spinal cord segments collected from animals that received GRNOPCl. Importantly however, human DNA was not detected in the CSF, as would be expected in this event.

The highest level detected in the spinal cord was measured in spinal cord section 6 of animal number 2013 (Day 14 GRNOPCl). Given that the value in this segment, 298.33 pg human DNA per microgram minipig gDNA, was higher than those calculated for the other animals in the spinal cord segment containing the injection site, and that the 2-3 spinal cord segments between the injection site and spinal cord section 6 have little to no measurable levels of human DNA, it is unlikely that this value is due exclusively to the presence of GRNOPCl at this level of spinal cord.

The additional use of a qPCR assay was intended to provide quantitative information regarding the biodistribution of GPOR-OPC1 after cervical administration. However, the qPCR data obtained from the study are ambiguous, due to apparent contamination of samples during tissue collection. Expression of human DNA in animals that received Cryostor vehicle only does not reflect persistence of GPOR-OPCl.

Very few animals in the study had human cells present at notable levels in the peripheral tissues sampled; indeed, it is likely restricted to only 1 or 2 animals that received GRNOPCl and were included in the Day 14 termination time point. Low numbers of human cell equivalents were calculated in the brainstem and pons/medulla of animals that received GRNOPCl in both the Day 2 and Day 14 time point groups, however, at levels lower than calculated in animals that received vehicle. The assay supports that the largest concentration of GRNOPCl remaining in the sampled tissues occurs in the cervical spinal cord, and levels are higher in animals that were terminated at Day 2 than in those that were terminated at Day 14. Most animals that received GRNOPCl also had human cell equivalents calculated in the most lumbar of the cervical spinal cord segments collected, although human DNA was also detected in tissue samples from the same level of spinal cord in animals that received HBSS. The data do not support extensive distribution of GRNOPCl after administration into the cervical spinal cord using clinical delivery methods.

### Results Summary

There were no effects of GPOR-OPC1 or LCTOPC1 administration, or Cryostor CS10 administration on mortality in the study.

GPOR-OPC1 and LCTOPC1 cell survival was confirmed by IHC.

4/8 animals administered GPOR-OPC1 test article had HuNu-positive cells identified—all 4 Day 2 animals had detectable cells.

5/8 animals administered LCTOPC1 test article had HuNu-positive cells identified-all 4 Day 2 animals had detectable cells.

There was no positive HuNu-positive staining in the transplantation sites from Day 2 and Day 14 vehicle-administered animals (4 animals, 8 total sites).

In all animals, test article cells were clustered in a single group at the site of injection and migration of the cells was not apparent.

Body weights were normal across the study period and were not affected by GPOR-OPC1 or LCTOPC1 administration.

There were no observed GPOR-OPC1 or LCTOPC1-related effects on hematology parameters on Day 2. In animals that received GPOR-OPC1 or LCTOPC1 and were included in the Day 14 group, moderate increases were measured in neutrophil counts, suggesting an on-going inflammatory response.

In addition to the presence of human cells in the Transplantation sites, the main additional microscopic findings were mononuclear cell infiltration, changes in spinal cord white matter (typically minimal to mild axonal swelling and dilation of the myelin space around axons), and acute hemorrhage (typically Day 2 samples and attributed to the recent transplantation procedure). The incidence and severity of changes these additional changes were generally higher in Day 14 sites compared to Day 2 sites.

No microscopic findings in the brain, lung, heart, spleen, liver, or kidney were interpreted as related to the test article administration. Microscopic findings in the spinal cord sections rostral and caudal to the transplantation sites were generally minimal and similar to those in the transplantation site.

The highest concentration of persistent GPOR-OPC1 and LCTOPC1 cells were detected at the level of the cervical spinal cord, as was confirmed histologically using ISH. Additional tissue samples that may contain test article cells include the pons/medulla, brainstem and the most lumbar spinal cord sections collected; levels of human DNA detected in animals that received test article cells were higher at 2 days than at 14 days post-injection. The data do not support an extensive distribution of GPOR-OPC1 or LCTOPC1 cells after administration into the cervical spinal cord.

### Conclusions

Either GPOR-OPC1 or LCTOPC1 were each administered by cervical intraspinal cord injection using the Parenchymal Spinal Delivery System (Lineage Cell Therapeutics, Inc.) into eight uninjured, immunosuppressed Yucatan minipigs at a dose level of 1×10⁷ cells (two separate injections).

Four uninjured immunosuppressed Yucatan minipigs received Cryostor vehicle using similar methods. Animals in both treatment groups were designated for terminal necropsy on Day 2 or Day 14. Overall, both GPOR-OPC1 and LCTOPC1 were well tolerated in all animals when compared to Cryostor control.

GPOR-OPC1 and LCTOPC1 cell survival was confirmed in all Day 2 animals that received test article by H&E and IHC evaluations. Human cells could be detected at 1 transplantation site in one Day 14 animal having received LCTOPC1 test article. Focal accumulations of GPOR-OPC1 and LCTOPC1 were present in the dorsal to dorsolateral horns of the spinal cord sections obtained near the level of cervical injection and were not identified in any other tissue sections taken along the entire rostrocaudal length of the spinal cord.

Neither GPOR-OPC1 nor LCTOPC1 were observed in the brain by H&E and IHC assessments.

There was no evidence of teratomatous tissue or ectopic tissue in the brain, spinal cord or collected peripheral tissues from any animal.

Administration of either GPOR-OPC1 or LCTOPC1 did not affect mortality rates; all animals survived to scheduled termination.

Evaluation of neurological motor function after spinal injections showed no detectable deficit in any animal. Normal motor function continued for the duration of the study (i.e., 2 days or 14 days) with all animals displaying normal fore limb-hind limb coordination and ambulatory function. No signs of spontaneous pain (presented by vocalization) were noted in any animal.

There were no observed systemic effects of GPOR-OPC1 or LCTOPC1 administration.

Minimal to moderate mild micropathological findings in the spinal cord, such as mononuclear cell infiltration, changes in spinal cord white matter (typically minimal to mild axonal swelling and dilation of the myelin space around axons), and acute hemorrhage (typically Day 2 samples and attributed to the recent transplantation procedure). The incidence and severity of changes these additional changes were generally higher in Day 14 sites compared to Day 2 sites. These were considered procedure-related and were not attributed to test article administration.

There were no significant differences in body weights, or hematological, coagulation or clinical chemistry findings.

Although sample contamination with human DNA was detected in the qPCR assay, the greatest concentration of persistent GRNOPCl occurred at the level of the cervical spinal cord. Additional tissue samples that may contain low levels of GRNOPCl include the pons/medulla, brainstem and the most lumbar spinal cord sections collected; however, GRNOPCl were confirmed histologically only in the cervical spinal cord.

Overall, both GPOR-OPC1 and LCTOPC1 cells were well tolerated in all animals when compared to Cryostor control.

### EMBODIMENTS

Embodiment P1. A method of cellular therapy comprising administering to a subject an OPC composition to improve one or more neurological functions in the subject.

Embodiment P2. The method of embodiment P1, wherein the OPC cell population is injected or implanted into the subject.

Embodiment P3. The method of embodiment P1 or P2, wherein the subject has a neurological injury due to spinal cord injury, stroke, or multiple sclerosis.

Embodiment 1. A method of improving one or more neurological functions in a subject having a spinal cord injury (SCI), the method comprising: administering to the subject a first dose of a composition comprising human pluripotent stem cell-derived oligodendrocyte progenitor cells (OPCs); and optionally administering two or more doses of the composition.

Embodiment 2. The method of embodiment 1, further comprising administering to the subject a second dose of the composition.

Embodiment 3. The method of embodiment 1, further comprising administering to the subject a third dose of the composition.

Embodiment 4. The method of any of embodiments 1-3, wherein each administration comprises injecting the composition into the spinal cord of the subject.

Embodiment 5. The method of any of embodiments 1-4, wherein the SCI is a subacute cervical SCI.

Embodiment 6. The method of any of embodiments 1-4, wherein the SCI is a chronic cervical SCI.

Embodiment 7. The method of any of embodiments 1-4, wherein the SCI is a subacute thoracic SCI.

Embodiment 8. The method of any of embodiments 1-4, wherein the SCI is a chronic thoracic SCI.

Embodiment 9. The method of any one of the preceding embodiments, wherein the first dose, second dose, and/or third dose of the composition comprises about 1×10⁶ to about 3×10⁷ OPC cells.

Embodiment 10. The method of any one of the preceding embodiments, wherein the first dose of the composition comprises about 2×10⁶ OPC cells.

Embodiment 11. The method of any one of the preceding embodiments, wherein the first dose or the second dose of the composition comprises about 1×10⁷ OPC cells.

Embodiment 12. The method of any one of the preceding embodiments, wherein the second dose or the third dose of the composition comprises about 2×10⁷ OPC cells.

Embodiment 13. The method of any one of the preceding embodiments, wherein each of the first dose, second dose, and third dose of the composition are administered about 20 to about 45 days after the SCI.

Embodiment 14. The method of any one of the preceding embodiments, wherein each of the first dose, second dose, and third dose of the composition are administered about 14 to about 90 days after the SCI.

Embodiment 15. The method of any one of the preceding embodiments, wherein each of the first dose, second dose, and third dose of the composition are administered about 14 to about 75 days after the SCI.

Embodiment 16. The method of any one of the preceding embodiments, wherein each of the first dose, second dose, and third dose of the composition are administered about 14 to about 60 days after the SCI.

Embodiment 17. The method of any one of the preceding embodiments, wherein each of the first dose, second dose, and third dose of the composition are administered about 14 to about 30 days after the SCI.

Embodiment 18. The method of any one of the preceding embodiments, wherein each of the first dose, second dose, and third dose of the composition are administered about 20 to about 75 days after the SCI.

Embodiment 19. The method of any one of the preceding embodiments, wherein each of the first dose, second dose, and third dose of the composition are administered about 20 to about 60 days after the SCI.

Embodiment 20. The method of any one of the preceding embodiments, wherein each of the first dose, second dose, and third dose of the composition are administered about 20 to about 40 days after the SCI.

Embodiment 21. The method of any one of the preceding embodiments, wherein each of the first dose, second dose, and third dose of the composition are administered between about 14 days after the SCI and the lifetime of the subject.

Embodiment 22. The method of any one of embodiments 2-21, wherein the injection is performed in a caudal half of an epicenter of the SCI.

Embodiment 23. The method of embodiment 22, wherein the injection is about 6 mm into the spinal cord of the subject.

Embodiment 24. The method of embodiment 22, wherein the injection is about 5 mm into the spinal cord of the subject.

Embodiment 25. A method of improving one or more neurological functions in a subject having a spinal cord injury (SCI), the method comprising: administering to the subject a dose of a composition comprising human pluripotent stem cell-derived oligodendrocyte progenitor cells (OPCs).

Embodiment 26. The method of embodiment 25, wherein the dose of the composition comprises about 1×10⁶ to about 3×10⁷ OPC cells.

Embodiment 27. The method of embodiment 26, wherein the dose of the composition comprises about 2×10⁶ OPC cells.

Embodiment 28. The method of any one of embodiments 25-27, wherein the administration of the composition comprises injecting the composition into the spinal cord of the subject.

Embodiment 29. The method of any one of embodiments 25-28, wherein the composition is administered about 7 to about 14 days after the SCI.

Embodiment 30. The method of any one of embodiments 25-29, wherein the injection is performed in a caudal half of an epicenter of the SCI.

Embodiment 31. The method of any one of embodiments 25-30, wherein the injection is about 6 mm into the spinal cord of the subject.

Embodiment 32. The method of any one of embodiments 25-30, wherein the injection is about 5 mm into the spinal cord of the subject.

Embodiment 33. The method of any one of embodiments 25-32 wherein the SCI is a subacute thoracic SCI.

Embodiment 34. The method of any one of embodiments 25-32 wherein the SCI is a chronic thoracic SCI.

Embodiment 35. The method of any one of embodiments 25-32 wherein the SCI is a subacute cervical SCI.

Embodiment 36. The method of any one of embodiments 25-32 wherein the SCI is a chronic cervical SCI.

Embodiment 37. The method of any one of the above embodiments, wherein improving one or more neurological functions comprises an improvement in ISNCSCI exam upper extremity motor score (UEMS).

Embodiment 38. The method of embodiment 37, where in the improvement in UEMS occurs within about 6 months, about 12 months, about 18 months, about 24 months or more after injection.

Embodiment 39. The method of embodiment 37 or 38, wherein the improvement is an increase in UEMS of at least 10%, compared to baseline.

Embodiment 40. The method of any one of the above embodiments, wherein improving one or more neurological functions comprises an improvement in lower extremity motor scores (LEMS).

Embodiment 41. The method of embodiment 40, where in the improvement in LEMS occurs within about 6 months, about 12 months, about 18 months, about 24 months or more after injection.

Embodiment 42. The method of embodiment 37 or 38, wherein the improvement is at least one motor level improvement.

Embodiment 43. The method of embodiment 37 or 38, wherein the improvement is at least two motor level improvement.

Embodiment 44. The method of any one of embodiments 37-43, wherein the improvement is on one side of the subject's body.

Embodiment 45. The method of any one of embodiments 37-43, wherein the improvement is on both sides of the subject's body.

Embodiment 46. The method of any one of the preceding embodiments, wherein the dose of the composition is administered about 14 to about 90 days after the SCI.

Embodiment 47. The method of any one of the preceding embodiments, wherein the dose of the composition is administered about 14 to about 75 days after the SCI.

Embodiment 48. The method of any one of the preceding embodiments, wherein the dose of the composition is administered about 14 to about 60 days after the SCI.

Embodiment 49. The method of any one of the preceding embodiments, wherein the dose of the composition is administered about 14 to about 30 days after the SCI.

Embodiment 50. The method of any one of the preceding embodiments, wherein the dose of the composition is administered about 20 to about 75 days after the SCI.

Embodiment 51. The method of any one of the preceding embodiments, wherein the dose of the composition is administered about 20 to about 60 days after the SCI.

Embodiment 52. The method of any one of the preceding embodiments, wherein the dose of the composition is administered about 20 to about 40 days after the SCI.

Embodiment 53. The method of any one of the preceding embodiments, wherein the dose of the composition is administered between about 14 days after the SCI and the lifetime of the subject.

Embodiment 54. A cell population comprising an increased proportion of cells positive for oligodendrocyte progenitor cell marker NG2 and reduced expression of non-OPC markers CD49f, CLDN6, and EpCAM, wherein the cell population is prepared according to the following method: culturing undifferentiated human embryonic stem cells (uhESC) in Glial Progenitor Medium comprising a MAPK/ERK inhibitor, a BMP signaling inhibitor, and Retinoic Acid to obtain glial-restricted cells; differentiating the glial-restricted cells into oligodendrocyte progenitor cells (OPCs) having an increased proportion of cells positive for oligodendrocyte progenitor cell marker NG2 and reduced expression of non-OPC markers CD49f, CLDN6, and EpCAM.

Embodiment 55. The cell population of embodiment 54, for use in treating a thoracic spinal cord injury (SCI) in a subject.

Embodiment 56. The cell population of embodiment 55, wherein the thoracic SCI is a subacute thoracic SCI.

Embodiment 57. The cell population of embodiment 55, wherein the thoracic SCI is a chronic thoracic SCI.

Embodiment 58. The cell population of embodiment 54, for use in treating a cervical spinal cord injury (SCI) in a subject.

Embodiment 59. The cell population of embodiment 58, wherein the cervical SCI is a subacute cervical SCI.

Embodiment 60. The cell population of embodiment 58, wherein the cervical SCI is a chronic cervical SCI.

Embodiment 61. The cell population of any one of embodiments 54-60, wherein the composition is administered via injection to the subject after the SCI.

Embodiment 62. The cell population of embodiment 61, wherein the injection is performed in a caudal half of an epicenter of the SCI.

Embodiment 63. The cell population of embodiment 61, wherein the injection is about 6 mm into the spinal cord of the subject.

Embodiment 64. The cell population of embodiment 61, wherein the injection is about 5 mm into the spinal cord of the subject.

Embodiment 65. The cell population of any one of embodiments 54-64, wherein the injection is performed about 14 to about 90 days after the SCI.

Embodiment 66. The cell population of any one of embodiments 54-64, wherein the injection is performed about 14 to about 75 days after the SCI.

Embodiment 67. The cell population of any one of embodiments 54-64, wherein the injection is performed about 14 to about 60 days after the SCI.

Embodiment 68. The cell population of any one of embodiments 54-64, wherein the injection is performed about 14 to about 30 days after the SCI.

Embodiment 69. The cell population of any one of embodiments 54-64, wherein the injection is performed about 20 to about 75 days after the SCI.

Embodiment 70. The cell population of any one of embodiments 54-64, wherein the injection is performed about 20 to about 60 days after the SCI.

Embodiment 71. The cell population of any one of embodiments 54-64, wherein the injection is performed about 20 to about 40 days after the SCI.

Embodiment 72. The cell population of any one of embodiments 54-64, wherein the injection is performed between about 14 days after the SCI and the lifetime of the subject.

Embodiment 73. A method of improving one or more neurological functions in a subject having a spinal cord injury (SCI), the method comprising: administering to the subject a first dose of the cell population of embodiment 54; administering to the subject a second dose of the cell population; and optionally administering to the subject a third dose of the cell population.

Embodiment 74. The method of embodiment 73, wherein the SCI is a subacute cervical SCI.

Embodiment 75. The method of embodiment 73, wherein the SCI is a chronic cervical SCI.

Embodiment 76. The method of embodiment 73, wherein the SCI is a subacute thoracic SCI.

Embodiment 77. The method of embodiment 73, wherein the SCI is a chronic thoracic SCI.

Embodiment 78. The method of any one of embodiments 73-77, wherein each of the first dose, second dose, and third dose of the composition are administered about 14 to about 90 days after the SCI.

Embodiment 79. The method of any one of embodiments 73-77, wherein each of the first dose, second dose, and third dose of the composition are administered about 14 to about 75 days after the SCI.

Embodiment 80. The method of any one of embodiments 73-77, wherein each of the first dose, second dose, and third dose of the composition are administered about 14 to about 60 days after the SCI.

Embodiment 81. The method of any one of embodiments 73-77, wherein each of the first dose, second dose, and third dose of the composition are administered about 14 to about 30 days after the SCI.

Embodiment 82. The method of any one of embodiments 73-77, wherein each of the first dose, second dose, and third dose of the composition are administered about 20 to about 75 days after the SCI.

Embodiment 83. The method of any one of embodiments 73-77, wherein each of the first dose, second dose, and third dose of the composition are administered about 20 to about 60 days after the SCI.

Embodiment 84. The method of any one of embodiments 73-77, wherein each of the first dose, second dose, and third dose of the composition are administered about 20 to about 40 days after the SCI.

Embodiment 85. The method of any one of embodiments 73-77, wherein each of the first dose, second dose, and third dose of the composition are administered between about 14 days after the SCI and the lifetime of the subject.

What is claimed is:

1. A method of improving one or more neurological functions in a subject having a spinal cord injury (SCI), the method comprising:
administering to the subject a first dose of a composition comprising human pluripotent stem cell-derived oligodendrocyte progenitor cells (OPCs); and optionally administering two or more doses of the composition.

2. The method of claim 1, further comprising administering to the subject a second dose of the composition.

3. The method of claim 1, further comprising administering to the subject a third dose of the composition.

4. The method of any of claims 1-3, wherein each administration comprises injecting the composition into the spinal cord of the subject.

5. The method of any of claims 1-4, wherein the SCI is a subacute cervical SCI.

6. The method of any of claims 1-4, wherein the SCI is a chronic cervical SCI.

7. The method of any of claims 1-4, wherein the SCI is a subacute thoracic SCI.

8. The method of any of claims 1-4, wherein the SCI is a chronic thoracic SCI.

9. The method of any one of the preceding claims, wherein the first dose, second dose, and/or third dose of the composition comprises about 1×10⁶ to about 3×10⁷ OPC cells.

10. The method of any one of the preceding claims, wherein the first dose of the composition comprises about 2×10⁶ OPC cells.

11. The method of any one of the preceding claims, wherein the first dose or the second dose of the composition comprises about 1×10⁷ OPC cells.

12. The method of any one of the preceding claims, wherein the second dose or the third dose of the composition comprises about 2×10⁷ OPC cells.

13. The method of any one of the preceding claims, wherein each of the first dose, second dose, and third dose of the composition are administered about 20 to about 45 days after the SCI.

14. The method of any one of the preceding claims, wherein each of the first dose, second dose, and third dose of the composition are administered about 14 to about 90 days after the SCI.

15. The method of any one of the preceding claims, wherein each of the first dose, second dose, and third dose of the composition are administered about 14 to about 75 days after the SCI.

16. The method of any one of the preceding claims, wherein each of the first dose, second dose, and third dose of the composition are administered about 14 to about 60 days after the SCI.

17. The method of any one of the preceding claims, wherein each of the first dose, second dose, and third dose of the composition are administered about 14 to about 30 days after the SCI.

18. The method of any one of the preceding claims, wherein each of the first dose, second dose, and third dose of the composition are administered about 20 to about 75 days after the SCI.

19. The method of any one of the preceding claims, wherein each of the first dose, second dose, and third dose of the composition are administered about 20 to about 60 days after the SCI.

20. The method of any one of the preceding claims, wherein each of the first dose, second dose, and third dose of the composition are administered about 20 to about 40 days after the SCI.

21. The method of any one of the preceding claims, wherein each of the first dose, second dose, and third dose of the composition are administered between about 14 days after the SCI and the lifetime of the subject.

22. The method of any one of claims 2-21, wherein the injection is performed in a caudal half of an epicenter of the SCI.

23. The method of claim 22, wherein the injection is about 6 mm into the spinal cord of the subject.

24. The method of claim 22, wherein the injection is about 5 mm into the spinal cord of the subject.

25. A method of improving one or more neurological functions in a subject having a spinal cord injury (SCI), the method comprising:
administering to the subject a dose of a composition comprising human pluripotent stem cell-derived oligodendrocyte progenitor cells (OPCs).

26. The method of claim 25, wherein the dose of the composition comprises about 1×10⁶ to about 3×10⁷ OPC cells.

27. The method of claim 26, wherein the dose of the composition comprises about 2×10⁶ OPC cells.

28. The method of any one of claims 25-27, wherein the administration of the composition comprises injecting the composition into the spinal cord of the subject.

29. The method of any one of claims 25-28, wherein the composition is administered about 7 to about 14 days after the SCI.

30. The method of any one of claims 25-29, wherein the injection is performed in a caudal half of an epicenter of the SCI.

31. The method of any one of claims 25-30, wherein the injection is about 6 mm into the spinal cord of the subject.

32. The method of any one of claims 25-30, wherein the injection is about 5 mm into the spinal cord of the subject.

33. The method of any one of claims 25-32 wherein the SCI is a subacute thoracic SCI.

34. The method of any one of claims 25-32 wherein the SCI is a chronic thoracic SCI.

35. The method of any one of claims 25-32 wherein the SCI is a subacute cervical SCI.

36. The method of any one of claims 25-32 wherein the SCI is a chronic cervical SCI.

37. The method of any one of the above claims, wherein improving one or more neurological functions comprises an improvement in ISNCSCI exam upper extremity motor score (UEMS).

38. The method of claim 37, where in the improvement in UEMS occurs within about 6 months, about 12 months, about 18 months, about 24 months or more after injection.

39. The method of claim 37 or 38, wherein the improvement is an increase in UEMS of at least 10%, compared to baseline.

40. The method of any one of the above claims, wherein improving one or more neurological functions comprises an improvement in lower extremity motor scores (LEMS).

41. The method of claim 40, where in the improvement in LEMS occurs within about 6 months, about 12 months, about 18 months, about 24 months or more after injection.

42. The method of claim 37 or 38, wherein the improvement is at least one motor level improvement.

43. The method of claim 37 or 38, wherein the improvement is at least two motor level improvement.

44. The method of any one of claims 37-43, wherein the improvement is on one side of the subject's body.

45. The method of any one of claims 37-43, wherein the improvement is on both sides of the subject's body.

46. The method of any one of the preceding claims, wherein the dose of the composition is administered about 14 to about 90 days after the SCI.

47. The method of any one of the preceding claims, wherein the dose of the composition is administered about 14 to about 75 days after the SCI.

48. The method of any one of the preceding claims, wherein the dose of the composition is administered about 14 to about 60 days after the SCI.

49. The method of any one of the preceding claims, wherein the dose of the composition is administered about 14 to about 30 days after the SCI.

50. The method of any one of the preceding claims, wherein the dose of the composition is administered about 20 to about 75 days after the SCI.

51. The method of any one of the preceding claims, wherein the dose of the composition is administered about 20 to about 60 days after the SCI.

52. The method of any one of the preceding claims, wherein the dose of the composition is administered about 20 to about 40 days after the SCI.

53. The method of any one of the preceding claims, wherein the dose of the composition is administered between about 14 days after the SCI and the lifetime of the subject.

54. A cell population comprising an increased proportion of cells positive for oligodendrocyte progenitor cell marker NG2 and reduced expression of non-OPC markers CD49f, CLDN6, and EpCAM, wherein the cell population is prepared according to the following method:
(i) culturing undifferentiated human embryonic stem cells (uhESC) in Glial Progenitor Medium comprising a MAPK/ERK inhibitor, a BMP signaling inhibitor, and Retinoic Acid to obtain glial-restricted cells;

55. (iii) differentiating the glial-restricted cells into oligodendrocyte progenitor cells (OPCs) having an increased proportion of cells positive for oligodendrocyte progenitor cell marker NG2 and reduced expression of non-OPC markers CD49f, CLDN6, and EpCAM.

56. The cell population of claim 54, for use in treating a thoracic spinal cord injury (SCI) in a subject.

57. The cell population of claim 55, wherein the thoracic SCI is a subacute thoracic SCI.

58. The cell population of claim 55, wherein the thoracic SCI is a chronic thoracic SCI.

59. The cell population of claim 54, for use in treating a cervical spinal cord injury (SCI) in a subject.

60. The cell population of claim 58, wherein the cervical SCI is a subacute cervical SCI.

61. The cell population of claim 58, wherein the cervical SCI is a chronic cervical SCI.

62. The cell population of any one of claims 54-60, wherein the composition is administered via injection to the subject after the SCI.

63. The cell population of claim 61, wherein the injection is performed in a caudal half of an epicenter of the SCI.

64. The cell population of claim 61, wherein the injection is about 6 mm into the spinal cord of the subject.

65. The cell population of claim 61, wherein the injection is about 5 mm into the spinal cord of the subject.

66. The cell population of any one of claims 54-64, wherein the injection is performed about 14 to about 90 days after the SCI.

67. The cell population of any one of claims 54-64, wherein the injection is performed about 14 to about 75 days after the SCI.

68. The cell population of any one of claims 54-64, wherein the injection is performed about 14 to about 60 days after the SCI.

69. The cell population of any one of claims 54-64, wherein the injection is performed about 14 to about 30 days after the SCI.

70. The cell population of any one of claims 54-64, wherein the injection is performed about 20 to about 75 days after the SCI.

71. The cell population of any one of claims 54-64, wherein the injection is performed about 20 to about 60 days after the SCI.

72. The cell population of any one of claims 54-64, wherein the injection is performed about 20 to about 40 days after the SCI.

73. The cell population of any one of claims 54-64, wherein the injection is performed between about 14 days after the SCI and the lifetime of the subject.

74. A method of improving one or more neurological functions in a subject having a spinal cord injury (SCI), the method comprising:
administering to the subject a first dose of the cell population of claim 54;
administering to the subject a second dose of the cell population; and optionally
administering to the subject a third dose of the cell population.

75. The method of claim 73, wherein the SCI is a subacute cervical SCI.

76. The method of claim 73, wherein the SCI is a chronic cervical SCI.

77. The method of claim 73, wherein the SCI is a subacute thoracic SCI.

78. The method of claim 73, wherein the SCI is a chronic thoracic SCI.

79. The method of any one of claims 73-77, wherein each of the first dose, second dose, and third dose of the composition are administered about 14 to about 90 days after the SCI.

80. The method of any one of claims 73-77, wherein each of the first dose, second dose, and third dose of the composition are administered about 14 to about 75 days after the SCI.

81. The method of any one of claims 73-77, wherein each of the first dose, second dose, and third dose of the composition are administered about 14 to about 60 days after the SCI.

82. The method of any one of claims 73-77, wherein each of the first dose, second dose, and third dose of the composition are administered about 14 to about 30 days after the SCI.

83. The method of any one of claims 73-77, wherein each of the first dose, second dose, and third dose of the composition are administered about 20 to about 75 days after the SCI.

84. The method of any one of claims 73-77, wherein each of the first dose, second dose, and third dose of the composition are administered about 20 to about 60 days after the SCI.

85. The method of any one of claims 73-77, wherein each of the first dose, second dose, and third dose of the composition are administered about 20 to about 40 days after the SCI.

86. The method of any one of claims 73-77, wherein each of the first dose, second dose, and third dose of the composition are administered between about 14 days after the SCI and the lifetime of the subject.

87. The method of any one of claim 1-53 or 74-86, wherein the composition is administered using a parenchymal spinal delivery (PSD) system.

88. The method of claim 87, wherein the PSD system comprises a device comprises a system as shown in any of FIGS. 39-41.

89. The method of claim 87, wherein the PSD system comprises a device comprising:
(a) a frame having an elongated body and a plurality of holders extending therefrom;
(b) a plurality of first magnets, each being fixedly attached to a holder;
(c) a tube having a first end and a second end, the tube being slidingly disposed within through-holes disposed in each holder and in each first magnet;
(d) a plurality of second magnets fixedly attached to an exterior surface of the tube; and
(e) a needle fixedly attached to the first end of the tube.

90. The method of claim 88 or 89, wherein each of the first magnets and the each of the second magnets are disposed such that a north pole of one first magnet faces a north pole of one second magnet or a south pole of one first magnet faces a south pole of one second magnet, thereby providing a magnetic repulsive force upon which the tube floats.

91. The method of any one of claims 88-90, wherein the PSD system further comprises a reservoir in fluid communication with the needle, the reservoir containing the composition.

92. The method of claim 91, wherein the PSD system further comprises a digital microinjector configured to control flow of the composition through the needle.

93. The method of any one of claims 87-92, wherein the device comprises a stopper adjacent to the needle.

94. The method of claim 93, wherein the stopper prevents the needle from travelling further into the spinal cord.

95. The method of any one of claims 87-94, wherein the subject is not removed from ventilation during administration.

96. The method of any one of claims 87-95, where administering the composition comprises:
(i) positioning the PSD system over the spinal cord of the subject;
(ii) lowering the needle into the spinal cord; and
(iii) delivering a dose of the composition to the spinal cord.

97. The method of claim 96, wherein the needle and tube of the device float due to magnetic repulsive forces within the device, thereby compensating for spinal cord pulsation.

98. The method of claim 96 or 97, wherein step (ii) comprises lowering the needle until the stopper rests on the spinal cord.

99. A parenchymal spinal delivery (PSD) system comprising (i) a device as shown in any of FIGS. 39-41 which comprises a needle; and
(ii) a reservoir in fluid communication with the needle, the reservoir containing a composition according to any one of claims 54-73.

100. The system of claim 99, wherein the device comprises:
(a) a frame having an elongated body and a plurality of holders extending therefrom;
(b) a plurality of first magnets, each being fixedly attached to a holder;
(c) a tube having a first end and a second end, the tube being slidingly disposed within through-holes disposed in each holder and in each first magnet;
(d) a plurality of second magnets fixedly attached to an exterior surface of the tube; and
(e) the needle fixedly attached to the first end of the tube.

101. The system of claim 100, wherein each of the first magnets and the each of the second magnets are disposed such that a north pole of one first magnet faces a north pole of one second magnet or a south pole of one first magnet faces a south pole of one second magnet, thereby providing a magnetic repulsive force upon which the tube floats.

102. The system of any one of claims 99-101, further comprising a microinjector or microinjection pump configured to control flow of the composition through the needle.

103. The system of any one of claims 99-102, further comprising an XYZ manipulator for manipulating a position of the needle.

104. The system of any one of claims 99-103, further comprising a stopper adjacent to the needle.

105. The system of claim 104, wherein the stopper prevents the needle from travelling further into a spinal cord.

106. A method for administering cells to a spinal cord of a patient comprising:
(i) positioning the system of any one of claims 99-105 over the spinal cord of the subject;
(ii) lowering the needle into the spinal cord; and
(iii) delivering a dose of the composition to the spinal cord.

107. The method of claim 106, wherein the needle and tube of the device float due to magnetic repulsive forces within the device, thereby compensating for spinal cord pulsation.

108. The method of claim 106 or 107, wherein step (ii) comprises lowering the needle until the stopper rests on the spinal cord.


SEQUENCE LISTING (3 sequences)

SEQ ID NO: 1 - DNA, 14 bp, synthetic construct
  source 1..14: mol_type = other DNA
ggcgcggtggctca

SEQ ID NO: 2 - DNA, 20 bp, synthetic construct
  source 1..20: mol_type = other DNA
gatctcggctcactgcaagc

SEQ ID NO: 3 - DNA, 27 bp, synthetic construct
  source 1..27: mol_type = other DNA
aggagatcgagaccatcctggctaaca